(19) United States
(12) Patent Application Publication
(10) Pub. No.: US 2023/0032846 A1
(43) Pub. Date: Feb. 2, 2023

(54) SYSTEMS AND METHODS FOR LIPID NANOPARTICLE DELIVERY OF GENE EDITING MACHINERY

(71) Applicant: Duke University, Durham, NC (US)

(72) Inventors: Charles A. Gersbach, Chapel Hill, NC (US); Christopher Nelson, Durham, NC (US)

(21) Appl. No.: 17/782,112

(22) PCT Filed: Dec. 3, 2020
(86) PCT No.: PCT/US2020/063150

§ 371 Date: Jun. 2, 2022

Related U.S. Application Data
(60) Provisional application No. 62/943,093, filed on Dec. 3, 2019.

(51) Int. Cl. A61K 48/00; C12N 15/90; C12N 15/11; C12N 9/22; A61K 47/69; A61K 9/51; A61P 21/00

(52) U.S. Cl. CPC A61K 48/0008; C12N 15/907; C12N 15/11; C12N 9/22; A61K 47/6929; A61K 9/5123; A61P 21/00; C12N 2310/20; C12N 2800/80; C12N 2310/315; C12N 2310/321

(57) ABSTRACT

The present invention provides DNA targeting systems and methods for delivery of gene editing machinery using lipid nanoparticles or microparticles.

### CROSS-REFERENCE TO RELATED APPLICATIONS

This application claims priority to U.S. Provisional Patent Application No. 62/943,093, filed Dec. 3, 2019, which is incorporated herein by reference in its entirety.

### SEQUENCE LISTING

This application includes a Sequence Listing which has been submitted electronically in ASCII format and is hereby incorporated by reference in its entirety. Said ASCII copy created on Dec. 3, 2020, is named “028193-9338-WO01_As_Filed_Sequence_Listing.txt” and is 144 Kbytes in size.

### TECHNICAL FIELD

The present disclosure relates to systems and methods for delivery of gene editing machinery using lipid nanoparticles or microparticles.

### INTRODUCTION

Hereditary genetic diseases have devastating effects on children in the United States. These diseases currently have no cure and can only be managed by attempts to alleviate the symptoms. For decades, the field of gene therapy has promised a cure to these diseases. However, technical hurdles regarding the safe and efficient delivery of therapeutic genes to cells and patients have limited this approach. Duchenne muscular dystrophy (DMD) is a fatal genetic disease, clinically characterized by muscle wasting, loss of ambulation, and death typically in the third decade of life due to the loss of functional dystrophin. DMD is the result of inherited or spontaneous mutations in the dystrophin gene. Most mutations causing DMD are a result of deletions of exon(s), pushing the translational reading frame out of frame.

CRISPR/Cas9-based gene editing systems can be used to introduce site-specific double strand breaks at targeted genomic loci. This DNA cleavage stimulates the natural DNA-repair machinery, leading to one of two possible repair pathways. In the absence of a donor template, the break will be repaired by non-homologous end joining (NHEJ), an error-prone repair pathway that leads to small insertions or deletions of DNA. This method can be used to intentionally disrupt, delete, or alter the reading frame of targeted gene sequences. However, if a donor template is provided along with the nucleases, then the cellular machinery will repair the break by homologous recombination, which is enhanced several orders of magnitude in the presence of DNA cleavage. This method can be used to introduce specific changes in the DNA sequence at target sites. Engineered nucleases have been used for gene editing in a variety of human stem cells and cell lines, and for gene editing in the mouse liver. However, the major hurdle for implementation of these technologies is delivery to particular tissues in vivo in a way that is effective, efficient, and facilitates successful genome modification.

In vivo gene therapies and gene editing approaches typically use viral vectors for gene delivery. These viral vectors are difficult and expensive to manufacture and cannot be used for patients who already have immune responses to these viruses. Furthermore, viral vectors may not be amenable to re-administration, and are be limited by other safety concerns. The herein described methods relate to the successful use of lipid nanoparticles or microparticles, a nonviral delivery vehicle, with non-viral CRISPR: mRNA encoding Cas9 and two gRNAs, to edit the dystrophin gene in a humanized mouse model of Duchenne muscular dystrophy and successfully restore dystrophin while avoiding the hazards of viral vector delivery, including a significant host response.

### SUMMARY

In an aspect, the disclosure relates to a lipid nanoparticle or microparticle comprising a DNA targeting system. The DNA targeting system may include at least one gRNA molecule; and/or a polynucleotide encoding a Cas9 protein. In some embodiments, the lipid nanoparticle or microparticle is for delivering the DNA targeting system to a muscle cell. In some embodiments, the at least one gRNA molecule targets a fragment of a mutant dystrophin gene.

In an aspect, the disclosure relates to a lipid nanoparticle or microparticle for delivering a DNA targeting system to a muscle cell, the DNA targeting system comprising at least one gRNA molecule targeting a fragment of a mutant dystrophin gene, and/or a polynucleotide encoding a Cas9 nuclease.

In some embodiments, the at least one gRNA molecule comprises a first gRNA molecule and a second gRNA molecule. In some embodiments, the polynucleotide encoding a Cas9 protein or nuclease is mRNA. In some embodiments, the first gRNA molecule and the second gRNA molecule each comprise a targeting domain, wherein the first gRNA molecule is encoded by a polynucleotide comprising a nucleotide sequence selected from SEQ ID NO: 1, SEQ ID NO: 3, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 37, SEQ ID NO: 41. SEQ ID NO: 83, or SEQ ID NO: 110 or a fragment or complement thereof or comprises a nucleotide sequence selected from SEQ ID NOs: 112-124 or a fragment or complement thereof, and the second gRNA molecule is encoded by a polynucleotide comprising a nucleotide sequence selected from SEQ ID NO: 2, SEQ ID NO: 4, SEQ ID NO: 5, SEQ ID NO: 6, SEQ ID NO: 18, SEQ ID NO: 17, SEQ ID NO: 18, SEQ ID NO: 19, SEQ ID NO: 38, SEQ ID NO: 42, SEQ ID NO: 84, or SEQ ID NO: 111 or a fragment or complement thereof or comprises a nucleotide sequence selected from SEQ ID NOs: 125-134 or a fragment or complement thereof, and wherein the first gRNA molecule and the second gRNA molecule comprise different targeting domains. In some embodiments, the first gRNA molecule comprises a targeting domain comprising the nucleotide sequence of SEQ ID NO: 110 or a fragment or complement thereof or comprises the nucleotide sequence of SEQ ID NO: 124 or a fragment or complement thereof and the second gRNA molecule comprise a targeting domain comprising the nucleotide sequence of SEQ ID NO: 111 or a fragment or complement thereof or comprises the nucleotide sequence of SEQ ID NO: 134 or a fragment or complement thereof. In some embodiments, the at least one gRNA and the polynucleotide encoding the Cas9 protein or nuclease are encapsulated in the same lipid nanoparticle or microparticle. In some embodiments, the at least one gRNA and the polynucleotide encoding the Cas9 protein or nuclease are each encapsulated in a separate lipid nanoparticle. In some embodiments, the lipid nanoparticle or microparticle is selected from the group consisting of solid lipid nanoparticle (SLN), nanostructured lipid carrier (NLC), lipid-drug conjugate (LDC) nanoparticle, lipid nanocapsule (LNC), polymer lipid hybrid nanoparticle (PLN), and solid lipid microparticle (SLM). In some embodiments, the lipid nanoparticle or microparticle is a solid lipid nanoparticle (SLN). In some embodiments, the lipid nanoparticle or microparticle is a nanostructured lipid carrier (NLC). In some embodiments, the lipid nanoparticle or microparticle is a lipid-drug conjugate (LDC) nanoparticle. In some embodiments, the lipid nanoparticle or microparticle is a lipid nanocapsule (LNC). In some embodiments, the lipid nanoparticle or microparticle is a polymer lipid hybrid nanoparticle (PLN). In some embodiments, the lipid nanoparticle or microparticle is a solid lipid microparticle (SLM). In some embodiments, the at least one gRNA molecule targets an exon selected from exons 1-8, 10, 11, 12, 14, 18-22, 43-59, and 61-66 of the mutant dystrophin gene, or an intron that flanks an exon selected from exons 1-8, 10, 11, 12, 14, 16-22, 43-59, and 61-86 of the mutant dystrophin gene. In some embodiments, the DNA targeting system further comprises a donor sequence that comprises an exon of the wild-type dystrophin gene or a functional equivalent thereof, and wherein the exon is selected from exons 1-8, 10, 11, 12, 14, 16-22, 43-59, and 61-86 of the wild-type dystrophin gene. In some embodiments, the at least one gRNA molecule targets two introns that flank exon 51 of a human dystrophin gene. In some embodiments, the DNA targeting system induces a first double strand break in a first intron flanking exon 51 of a human dystrophin gene and a second double strand break in a second intron flanking exon 51 of a human dystrophin gene. In some embodiments, the polynucleotide encodes SpCas9 or SaCas9. In some embodiments, the mRNA is a modified mRNA. In some embodiments, the modified mRNA comprises one or more modifications selected from an N terminal NLS, a C terminal NLS, an HA Tag, and a uridine substitution. In some embodiments, the muscle cell is selected from a skeletal muscle cell, a cardiac muscle cell, and a smooth muscle cell.

In a further aspect, the disclosure relates to a composition comprising the lipid nanoparticle or microparticle as detailed herein and a pharmaceutically acceptable carrier.

Another aspect of the disclosure provides a method of treating Duchenne Muscular Dystrophy in a subject. The method may include administering to the subject a lipid nanoparticle or microparticle as detailed herein or a composition as detailed herein. In some embodiments, the subject experiences no or a limited humoral response that is cross reactive to the Cas9 protein or nuclease after administration. In some embodiments, the subject comprises a mutant dystrophin gene.

Another aspect of the disclosure provides a method of genome editing a mutant dystrophin gene in a subject. The method may include administering to the subject a lipid nanoparticle or microparticle as detailed herein or a composition as detailed herein.

In some embodiments, the mutant dystrophin gene comprises a premature stop codon, a disrupted reading frame, an aberrant splice acceptor site, or an aberrant splice donor site, or a combination thereof. In some embodiments, the mutant dystrophin gene comprises a frameshift mutation that causes a premature stop codon and a truncated gene product. In some embodiments, the mutant dystrophin gene comprises a deletion of one or more exons that disrupts the reading frame. In some embodiments, genome editing of the mutant dystrophin gene comprises a deletion of a premature stop codon, correction of a disrupted reading frame, modulation of splicing by disruption of a splice acceptor site, modulation of splicing by disruption of a splice donor sequence, deletion of exon 51, or a combination thereof. In some embodiments, the mutant dystrophin gene is edited by homology-directed repair. In some embodiments, dystrophin expression in the subject is increased by at least 1%, 2%, 5%, 10%, 15%, 20%, 25%, 30%, 35%, 40%, 45%, or at least 50% after editing. In some embodiments, the lipid nanoparticle or microparticle is administered to the subject before birth or within 1-2 days of birth. In some embodiments, the lipid nanoparticle or microparticle is administered to the subject intramuscularly, intravenously, or a combination thereof. In some embodiments, administration of the lipid nanoparticle or the microparticle or the compositions leads to expression of a functional or partially-functional dystrophin protein in the subject.

Another aspect of the disclosure provides a kit comprising the lipid nanoparticle or microparticle described herein.

The disclosure provides for other aspects and embodiments that will be apparent in light of the following detailed description and accompanying figures.

### BRIEF DESCRIPTION OF THE DRAWINGS

FIG. 1 shows the results of an ELISA against SpCas9 antibody, demonstrating a humoral response against SpCas9 enzyme after injection of RNPs but not against an mRNA encoding SpCas9.

FIG. 2 shows that local administration of mRNA was able to delete exon 51 from hDMD/d52 mice but RNP was not.

FIG. 3 shows that the deletion of exon 51 with local administration of mRNA restores expression of dystrophin.

FIG. 4 shows that mRNA injection did not lead to a strong humoral response. RNP administration raised Cas9 antibodies in both local and systemic injections.

### DETAILED DESCRIPTION

As described herein, certain DNA targeting systems and methods have been discovered to be effective for altering the expression of genes, genome engineering, and correcting or reducing the effects of mutations in genes involved in genetic diseases. The lipid nanoparticle or microparticle delivery of a CRISPR/Cas9-based system involves an mRNA encoding a Cas9 protein and at least one guide RNA encapsulated in one or more lipid nanoparticles or microparticles. In particular, the present disclosure describes a DNA targeting system that combines the DNA sequence targeting function of the CRISPR/Cas9-based system for delivery in one or more lipid nanoparticles microparticles, thus allowing changes in gene expression and/or epigenetic status via a non-viral delivery system. The system and methods may also be used in genome engineering and correcting or reducing the effects of gene mutations.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the singular forms “a,” “an,” and “the” include plural referents unless the context clearly dictates otherwise. Thus, for example, reference to “an intron” includes more than one intron, a reference to “a cell” includes a plurality of such cells, and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimize.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, Tex.; SAS Institute Inc., Cary, N.C.). The healthy or normal levels or ranges for a target or for a protein activity may be defined in accordance with standard practice. A control may be an subject or cell without an agonist as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Correcting”, “genome editing.” and “restoring” as used herein refers to changing a mutant gene that encodes a truncated protein or no protein at all, such that a full-length functional or partial-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Donor DNA”, “donor template,” “donor sequence,” and “repair template” as used interchangeably herein refers to a double-stranded DNA fragment or molecule that includes at least a portion of the gene of interest. The donor DNA may encode a full-functional protein or a partially functional protein. The donor sequence may include a fragment of a wild-type sequence encoding a protein. In some embodiments, the donor sequence comprises an exon of a wild-type dystrophin gene or a functional equivalent thereof, such as, for example, wherein the exon is selected from exons 1-8, 10, 11, 12, 14, 16-22, 43-59, and 61-66 of the wild-type dystrophin gene.

“Duchenne Muscular Dystrophy” or “DMD” as used interchangeably herein refers to a recessive, fatal, X-linked disorder that results in muscle degeneration and eventual death. DMD is a common hereditary monogenic disease and occurs in 1 in 3500 males. DMD is the result of inherited or spontaneous mutations that cause nonsense or frame shift mutations in the dystrophin gene. The majority of dystrophin mutations that cause DMD are deletions of exons that disrupt the reading frame and cause premature translation termination in the dystrophin gene. DMD patients typically lose the ability to physically support themselves during childhood, become progressively weaker during the teenage years, and die in their twenties.

“Dystrophin” as used herein refers to a rod-shaped cytoplasmic protein that is a part of a protein complex that connects the cytoskeleton of a muscle fiber to the surrounding extracellular matrix through the cell membrane. Dystrophin provides structural stability to the dystroglycan complex of the cell membrane that is responsible for regulating muscle cell integrity and function. The dystrophin gene or “DMD gene” as used interchangeably herein is 2.2 megabases at locus Xp21. The primary transcription measures about 2,400 kb with the mature mRNA being about 14 kb, 79 exons code for the protein, which is over 3500 amino acids in length.

“Encapsulated” as used herein refers to refers to a lipid nanoparticle that provides the mRNA or gRNA with full encapsulation, partial encapsulation, or both. In an embodiment, the nucleic acid (e.g., mRNA or gRNA) is fully encapsulated in the lipid nanoparticle or microparticle.

“Exon 51” as used herein refers to the exon 51 of the dystrophin gene. Exon 51 is frequently adjacent to frame-disrupting deletions in DMD patients and has been targeted in clinical trials for oligonucleotide-based exon skipping. A clinical trial for the exon 51 skipping compound eteplirsen recently reported a significant functional benefit across 48 weeks, with an average of 47% dystrophin positive fibers compared to baseline. Mutations in exon 51 are ideally suited for permanent correction by NHEJ-based genome editing.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional (either full or partially) protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a nucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operably linked to a coding sequence that encodes a protein such that when present in the cell of the individual, the coding sequence will be expressed.

“Genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but not limited to DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease.

“Genome editing” or “gene editing” as used herein refers to changing a gene. Genome editing may include correcting or restoring a mutant gene. Genome editing may include knocking out a gene, such as a mutant gene or a normal gene. Genome editing may be used to treat disease or enhance muscle repair by changing the gene of interest. In some embodiments, the compositions and methods detailed herein are for use in somatic cells and not germ line cells.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Lipid nanoparticle” or “LNP” as used herein is defined as a particle formed by lipid component, having at least one dimension on the order of nanometers (e.g., 1-1,000 nm). The LNP, in some embodiments, encapsulates a therapeutic agent. The therapeutic agent includes but is not limited to a nucleic acid molecule, a compound, a viral particle, a protein, or a peptide. In one embodiment, the LNP encapsulates one or more nucleic acid molecules. The term “microparticle” refers to a particle having at least one dimension from 10 nm to about 200 microns.

The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or after the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible

“Normal gene” as used herein refers to a gene that has not undergone a change, such as a loss, gain, or exchange of genetic material. The normal gene undergoes normal gene transmission and gene expression.

“Nuclease mediated NHEJ” as used herein refers to NHEJ that is initiated after a nuclease, such as a cas9, cuts double stranded DNA.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at a location not normally found in the wild-type gene. A premature stop codon may cause a protein to be truncated or shorter compared to the full-length version of the protein.

“Promoter” as used herein means a synthetic or naturally-derived molecule that is capable of conferring, activating, or enhancing expression of a nucleic acid in a cell. A promoter may comprise one or more specific transcriptional regulatory sequences to further enhance expression and/or to after the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, and the CMV IE promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Repeat variable diresidue” or “RVD” as used interchangeably herein refers to a pair of adjacent amino acid residues within a DNA recognition motif (also known as “RVD module”), which includes 33-35 amino acids, of a TALE DNA-binding domain. The RVD determines the nucleotide specificity of the RVD module. RVD modules may be combined to produce an RVD array. The “RVD array length” as used herein refers to the number of RVD modules that corresponds to the length of the nucleotide sequence within the TALEN target region that is recognized by a TALEN, i.e., the binding region.

“Site-specific nuclease” as used herein refers to an enzyme capable of specifically recognizing and cleaving DNA sequences. The site-specific nuclease may be engineered. Examples of engineered site-specific nucleases include zinc finger nucleases (ZFNs), TAL effector nucleases (TALENs), and CRISPR/Cas9-based systems.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment.

“Target gene” as used herein refers to any nucleotide sequence encoding a known or putative gene product. The target gene may be a mutated gene involved in a genetic disease.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Transcriptional regulatory elements” or “regulatory elements” refers to a genetic element which can control the expression of nucleic acid sequences, such as activate, enhancer, or decrease expression, or alter the spatial and/or temporal expression of a nucleic acid sequence. Examples of regulatory elements include, for example, promoters, enhancers, splicing signals, polyadenylation signals, and termination signals. A regulatory element can be “endogenous,” “exogenous,” or “heterologous” with respect to the gene to which it is operably linked. An “endogenous” regulatory element is one which is naturally linked with a given gene in the genome. An “exogenous” or “heterologous” regulatory element is one which is not normally linked with a given gene but is placed in operable linkage with a gene by genetic manipulation.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. DNA Targeting System for Genome Editing

The present invention is directed to DNA targeting systems for genome editing, genomic alteration, or altering gene expression. In a preferred embodiment the invention is directed to a DNA targeting system for the editing, genomic alteration, or altering gene expression of a dystrophin gene (e.g., human dystrophin gene). The DNA targeting system includes at least one gRNA molecule that targets a gene sequence and/or a polynucleotide encoding a Cas9 nuclease. The DNA targeting system is encapsulated in one or more lipid nanoparticle or microparticle. In some embodiments, the gRNAs of the DNA targeting system can target intronic regions surrounding exon 51 of the human dystrophin gene, causing genomic deletions of this region in order to restore expression of functional dystrophin in cells from DMD patients.

### 3. Dystrophin Gene

Dystrophin is a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane. The dystrophin gene is 2.2 megabases at locus Xp21. The primary transcription measures about 2,400 kb with the mature mRNA being about 14 kb, 79 exons code for the protein which is over 3500 amino acids. Normal skeleton muscle tissue contains only small amounts of dystrophin but its absence of abnormal expression leads to the development of severe symptoms. Some mutations in the dystrophin gene lead to the production of defective dystrophin and severe dystrophic phenotype in affected patients. Some mutations in the dystrophin gene lead to partially-functional dystrophin protein and a much milder dystrophic phenotype in affected patients.

DMD is the result of inherited or spontaneous mutations that cause nonsense or frame shift mutations in the dystrophin gene. Naturally occurring mutations and their consequences are relatively well understood for DMD. It is known that in-frame deletions that occur in the exon 45-55 regions (e.g., exon 51) contained within the rod domain can produce highly functional dystrophin proteins, and many carriers are asymptomatic or display mild symptoms. Furthermore, more than 60% of patients may theoretically be treated by targeting exons in this region of the dystrophin gene (e.g., targeting exon 51). Efforts have been made to restore the disrupted dystrophin reading frame in DMD patients by skipping non-essential exon(s) (e.g., exon 51 skipping) during mRNA splicing to produce internally deleted but functional dystrophin proteins. The deletion of internal dystrophin exon(s) (e.g., deletion of exon 51) retains the proper reading frame but cause the less severe Becker muscular dystrophy, or BMD. The Becker muscular dystrophy, or BMD, genotype is similar to DMD in that deletions are present in the dystrophin gene. However, these deletions leave the reading frame intact. Thus, an internally truncated but partially functional dystrophin protein is created. BMD has a wide array of phenotypes, but often if deletions are between exons 45-55 of dystrophin the phenotype is much milder compared to DMD. Thus, changing a DMD genotype to a BMD genotype is a common strategy to correct dystrophin. There are many strategies to correct dystrophin, many of which rely on restoring the reading frame of the endogenous dystrophin. This shifts the disease genotype from DMD to Becker muscular dystrophy. Many BMD patients have intragenic deletions that maintain the translational reading frame, leading to a shorter but largely functional dystrophin protein.

In certain embodiments, modification of exon 51 (e.g., deletion or excision of exon 51 by, e.g., NHEJ) to restore reading frame ameliorates the phenotype DMD subjects, including DMD subjects with deletion mutations. In certain embodiments, exon 51 of a dystrophin gene refers to the exon 51of the dystrophin gene. Exon 51 is frequently adjacent to frame-disrupting deletions in DMD patients and has been targeted in clinical trials for oligonucleotide-based exon skipping. A clinical trial for the exon 51 skipping compound eteplirsen reported a significant functional benefit across 48 weeks, with an average of 47% dystrophin positive fibers compared to baseline. Mutations in exon 51 are ideally suited for permanent correction by NHEJ-based genome editing.

Thus, this disclosure provides a DNA targeting system that comprises a lipid nanoparticle or microparticle as detailed herein. The lipid nanoparticle or microparticle can carry a DNA targeting system that generates a cleavage in the dystrophin gene, e.g., the human dystrophin gene. In certain embodiments, the DNA targeting system is configured to form two double stand breaks (a first double strand break and a second double strand break) in two introns (a first intron and a second intron) flanking a target position of the dystrophin gene, thereby deleting a segment of the dystrophin gene comprising the dystrophin target position. Deletion of the dystrophin exonic target position can optimize the dystrophin sequence of a subject suffering from Duchenne muscular dystrophy, e.g., it can increase the function or activity of the encoded dystrophin protein, or results in an improvement in the disease state of the subject. In certain embodiments, excision of the dystrophin exonic target position restores the dystrophin reading frame. The dystrophin exonic target position can comprise one or more exons of the dystrophin gene. In certain embodiments, the dystrophin target position comprises exon 51 of the dystrophin gene (e.g., human dystrophin gene).

A presently disclosed lipid nanoparticle or microparticle can mediate highly efficient gene editing at exon 51 of a dystrophin gene (e.g., the human dystrophin gene). The presently disclosed lipid nanoparticle or microparticle restores dystrophin protein expression in cells from DMD patients. Exon 51 is frequently adjacent to frame-disrupting deletions in DMD. Elimination of exon 51 from the dystrophin transcript by exon skipping can be used to treat approximately 15% of all DMD patients. This class of dystrophin mutations is ideally suited for permanent correction by NHEJ-based genome editing and HDR. The lipid nanoparticle or microparticle described herein has been developed for targeted modification of exon 51 in the human dystrophin gene. A presently disclosed lipid nanoparticle or microparticle is administered to human DMD cells and mediates efficient gene modification and conversion to the correct reading frame. In various aspects of the invention, dystrophin expression is increased by at least 1%, 2%, 5%, 10%, 15%, 20%, 25%, 30%, 35%, 40%, 45%, or at least 50%.

### 4. CRISPR System

A presently disclosed DNA targeting system that comprises the lipid nanoparticle or microparticle may provide the components for a CRISPR/Cas9-based gene editing system that is specific for a target gene, including but not limited to the dystrophin gene (e.g., human dystrophin gene). “Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a ‘memory’ of past exposures. Cas9 forms a complex with the 3′ end of the sgRNA (also referred interchangeably herein as “gRNA”), and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase Ill. This cleavage is followed by a second cleavage event within each spacer by Cas9, producing mature crRNAs that remain associated with the tracrRNA and Cas9, forming a Cas9:crRNA-tracrRNA complex.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Type II systems have differing PAM requirements. The S. pyogenes CRISPR system may have the PAM sequence for this Cas9 (SpCas9) as 5′-NRG-3′, where R is either A or G, and characterized the specificity of this system in human cells. A unique capability of the presently disclosed DNA targeting system is the straightforward ability to simultaneously target multiple distinct genomic loci by co-expressing a single Cas9 protein with two or more sgRNAs. For example, the Streptococcus pyogenes Type II system naturally prefers to use an “NGG” sequence, where “N” can be any nucleotide, but also accepts other PAM sequences, such as “NAG” in engineered systems (Hsu et al., Nature Biotechnology (2013) doi:10.1038/nbt.2647). Similarly, the Cas9 derived from Neisseria meningitidis (NmCas9) normally has a native PAM of NNNNGATT, but has activity across a variety of PAMs, including a highly degenerate NNNNGNNN PAM (Esvelt et al. Nature Methods (2013) doi:10.1038/nmeth.2681).

A Cas9 molecule of S. aureus recognizes the sequence motif NNGRR (R=A or G) (SEQ ID NO: 22) and directs cleavage of a target nucleic acid sequence 1 to 10. e.g., 3 to 5, bp upstream from that sequence. In certain embodiments, a Cas9 molecule of S. aureus recognizes the sequence motif NNGRRN (R=A or G) (SEQ ID NO: 23) and directs cleavage of a target nucleic acid sequence 1 to 10, e.g., 3 to 5, bp upstream from that sequence. In certain embodiments, a Cas9 molecule of S. aureus recognizes the sequence motif NNGRRT (R=A or G) (SEQ ID NO: 24) and directs cleavage of a target nucleic acid sequence 1 to 10, e.g., 3 to 5, bp upstream from that sequence. In certain embodiments, a Cas9 molecule of S. aureus recognizes the sequence motif NNGRRV (R=A or G) (SEQ ID NO: 25) and directs cleavage of a target nucleic acid sequence 1 to 10, e.g., 3 to 5, bp upstream from that sequence. In the aforementioned embodiments, N can be any nucleotide residue. e.g., any of A, G, C, or T. Cas9 molecules can be engineered to alter the PAM specificity of the Cas9 molecule.

### 5. CRISPR/Cas9-Based Gene Editing System

An engineered form of the Type II effector system of Streptococcus pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are DNA targeting systems for use in genome editing and treating genetic diseases. The presently disclosed DNA targeting system can be designed to target any gene, including genes involved in a genetic disease, aging, tissue regeneration, or wound healing. The DNA targeting system includes a polynucleotide encoding a Cas9 protein or a Cas9 fusion protein and one or more gRNAs. In some embodiments, the polynucleotide encoding a Cas9 protein or a Cas9 fusion protein is a mRNA. The mRNA may be a modified mRNA. A modified mRNA may include one or more modifications selected from an N terminal NLS, a C terminal NLS, an HA Tag, and a uridine substitution. The Cas9 fusion protein may, for example, include a domain that has a different activity that what is endogenous to Cas9, such as a transactivation domain.

The target gene (e.g., a dystrophin gene, e.g., human dystrophin gene) can be involved in differentiation of a cell or any other process in which activation of a gene can be desired, or can have a mutation such as a frameshift mutation or a nonsense mutation. If the target gene has a mutation that causes a premature stop codon, an aberrant splice acceptor site or an aberrant splice donor site, the DNA targeting system can be designed to recognize and bind a nucleotide sequence upstream or downstream from the premature stop codon, the aberrant splice acceptor site or the aberrant splice donor site. The DNA targeting system can also be used to disrupt normal gene splicing by targeting splice acceptors and donors to induce skipping of premature stop codons or restore a disrupted reading frame. The DNA targeting system may or may not mediate off-target changes to protein-coding regions of the genome.

a. Cas9 Molecules and Cas9 Fusion Proteins

The DNA targeting system of the invention comprises mRNA encoding a Cas9 protein or a Cas9 fusion protein. Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneumonias, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirllum, Clostridium cellulolyticum, Clostridium perfiingens, Corynebacterium accolens, Corynebacterium diphtheria, Corynebactenum matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In certain embodiments, the Cas9 molecule is a Streptococcus pyogenes Cas9 molecule (also referred herein as “SpCas9”). In certain embodiments, the Cas9 molecule is a Staphylococcus aureus Cas9 molecule (also referred herein as “SaCas9”). In some embodiments, the Cas9 molecule is a mutant Cas9 molecule. The Cas9 protein can be mutated so that the nuclease activity is inactivated. In some embodiments, the Cas9 molecule is a deactivated or inactivated Cas9 protein (dCas9 or iCas9), with no endonuclease activity. Exemplary mutations with reference to the S. pyogenes Cas9 sequence to inactivate the nuclease activity include: D10A, E762A, H840A, N854A, N863A and/or D986A. Exemplary mutations with reference to the S. aureus Cas9 sequence to inactivate the nuclease activity include D10A and N580A.

The mRNA encoding a Cas9 molecule can be a synthetic nucleic acid sequence. For example, the synthetic nucleic acid molecule can be chemically modified. The synthetic nucleic acid sequence can be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. In various embodiments of the invention there is limited or no humoral response that is cross reactive to Cas9 after administration to a subject.

Additionally or alternatively, the mRNA encoding a Cas9 molecule or Cas9 polypeptide is a modified mRNA. In one embodiment, the mRNA encoding a Cas9 molecule or Cas9 polypeptide may comprise a nuclear localization sequence (NLS). Nuclear localization sequences are known in the art. The NLS can be an N terminal NLS or a C terminal NLS. The mRNA encoding the Cas9 molecule or Cas9 polypeptide can include other modifications, including, but not limited to an HA Tag or a uridine substitution.

Alternatively or additionally, the DNA targeting system can include a fusion protein. The fusion protein can comprise two heterologous polypeptide domains, wherein the first polypeptide domain comprises a Cas protein and the second polypeptide domain has an activity such as transcription activation activity, transcription repression activity, transcription release factor activity, histone modification activity, nuclease activity, nucleic acid association activity, methylase activity, or demethylase activity. The fusion protein can include a Cas9 protein or a mutated Cas9 protein, fused to a second polypeptide domain that has an activity such as transcription activation activity, transcription repression activity, transcription release factor activity, histone modification activity, nuclease activity, nucleic acid association activity, methylase activity, or demethylase activity. In some embodiments, the second polypeptide domain comprises VP16 protein, multiple VP16 proteins, such as a VP48 domain or VP64 domain, p65 domain of NF kappa B transcription activator activity, p300 such as p300-full or p300-core, KRAB, and/or Tet1.

b. gRNA Targeting the Dystrophin Gene

The DNA targeting system includes one or more gRNA molecules. In one embodiment, the system comprises at least one gRNA molecule. The gRNA provides the targeting of the system. The gRNA is a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to cleave the target nucleic acid. The “target region”, “target sequence” or “protospacer” as used interchangeably herein refers to the region of the target gene (e.g., a dystrophin gene) to which the system targets. The DNA targeting system may include at least one gRNAs, wherein the gRNAs target different DNA sequences. The target DNA sequences may be overlapping. The target sequence or protospacer is followed by a PAM sequence at the 3′ end of the protospacer. Different Type II systems have differing PAM requirements. For example, the Streptococcus pyogenes Type II system uses an “NGG” sequence, where “N” can be any nucleotide. In some embodiments, the PAM sequence may be “NGG”, where “N” can be any nucleotide. In some embodiments, the PAM sequence may be NNGRRT (SEQ ID NO: 24) or NNGRRV (SEQ ID NO: 25).

The number of gRNA molecule encoded by a presently disclosed DNA targeting system can be one gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. In certain embodiments, the DNA targeting system encodes two gRNA molecules, i.e., a first gRNA molecule and a second gRNA molecule.

The gRNA molecule comprises a targeting domain, which is a complementary polynucleotide sequence of the target DNA sequence followed by a PAM sequence. The gRNA may comprise a “G” at the 5′ end of the targeting domain or complementary polynucleotide sequence. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule is 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The gRNA may target a region of the dystrophin gene (DMD). In certain embodiments, the gRNA can target at least one of exons, introns, the promoter region, the enhancer region, and/or the transcribed region of the dystrophin gene. In some embodiments, the gRNA targets at least one or more of exons 2, 3, 4, 5, 6, 7, 8, 11, 12, 17, 18, 19, 20, 21, 22, 23, 43, 44, 45, 46, 47, 48, 49, 50, 51, 52, 53, 54, 55, 56, 57, 58, 59, 61, 62, 63, 66, or 75 of the dystrophin gene. In some embodiments, the gRNA targets at least one or more of introns that flank exons 3, 4, 5, 51, 45, 53, 44, 46, 52, 50, 43, 8, 55, 2, 17, 7, 18, 21, 20, 12, 22, 19, 54, 59, 56, 11, 6, 57, 61, 66, 63, 62, 58, or 75 of the dystrophin gene. In some embodiments, the gRNA targets at least one or more of exons 43, 44, 45, 46, 47, 48, 49, 50, 51, 52, 53, 54, or 55 of the dystrophin gene. In some embodiments, the gRNA targets at least one or more of introns that flank exons 3, 4, 5, 43, 44, 45, 46, 47, 48, 49, 50, 51, 52, 53, 54, or 55 of the dystrophin gene. In some embodiments, the gRNA targets one or more of exon 23, exon 45, exon 50, exon 51, exons 45-55, exons 52-53, and exon 53 of the dystrophin gene. In some embodiments, the guide RNA targets the exon 45-55 hotspot of the dystrophin gene. In certain embodiments, the gRNA molecule targets intron 50 of the human dystrophin gene. In certain embodiments, the gRNA molecule targets intron 51 of the human dystrophin gene. In certain embodiments, the gRNA molecule targets exon 51 of the human dystrophin gene. The gRNA may include a targeting domain that comprises a nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 37, SEQ ID NO: 38, SEQ ID NO: 41, SEQ ID NO: 42, SEQ ID NO: 83, SEQ ID NO: 84. SEQ ID NO: 110, SEQ ID NO: 111, or a complement or fragment thereof. A fragment is any shorter segment of a reference sequence. A fragment may be, for example, 1, 2, 3, 4, 5, 6, 7, 8, 9, 10, 11, 12, 13, 14, or 15 nucleotides shorter than the reference sequence. In some embodiments, the gRNA is encoded by a polynucleotide selected from SEQ ID NOs: 1-19, 37-38, 41-42, 83-84, 110-111, or a fragment or a complement thereof. In some embodiments, the gRNA comprises a polynucleotide selected from SEQ ID NOs: 112-134, or a fragment or a complement thereof. In some embodiments, the first gRNA is encoded by a polynucleotide selected from SEQ ID NOs: 1, 3, 7-15, 37, 41, 83, and 110, or a fragment or a complement thereof, and the second gRNA is encoded by a polynucleotide selected from SEQ ID NOs: 2, 4-6, 16-19, 38, 42, 84, and 111, or a fragment or a complement thereof. In some embodiments, the first gRNA comprises a polynucleotide selected from SEQ ID NOs: 112-124, or a fragment or a complement thereof, and the second gRNA comprises a polynucleotide selected from SEQ ID NOs: 125-134, or a fragment or a complement thereof. In some embodiments, the at least one gRNA molecule targets an exon selected from exons 1-8, 10, 11, 12, 14, 16-22, 43-59, and 61-66 of the mutant dystrophin gene, or an intron that flanks an exon selected from exons 1-8, 10, 11, 12, 14, 16-22, 43-59, and 61-66 of the mutant dystrophin gene.

Single gRNA or multiplexed gRNAs can be designed to restore the dystrophin reading frame by targeting the mutational hotspot at exon 51 or and introducing either intraexonic small insertions and deletions, or excision of exon 51. In one embodiment the first gRNA comprises GATTGGCTTTGATTTCCCTA (SEQ ID NO: 110) and the second gRNA comprises GCAGTTGCCTAAGAACTGGT (SEQ ID NO: 111) to target exon 51. Following treatment with a presently disclosed DNA targeting system, dystrophin expression can be restored in Duchenne patient muscle cells in vitro.

6. Lipid Nanoparticle or Microparticles

The disclosed DNA targeting system may be encapsulated in one or more lipid nanoparticles or microparticles.

Lipid nanoparticles or microparticles are generally composed of an ionizable cationic lipid and 3 or more additional components, typically cholesterol, DOPE and a Polyethylene Glycol (PEG) containing lipid. The cationic lipid can bind to the positively charged nucleic acids (gRNA or mRNA) forming a dense complex that protects the nucleic acids from degradation. The components self-assemble to form particles in the size range of 1-1,000 nM in which the gRNA and/or polynucleotide is encapsulated in the core complexed with the cationic lipid and surrounded by a lipid bilayer like structure. After injection into the subject, these particles are taken up by the cell and the lipid nanoparticles or microparticles deliver the nucleic acids into the cytoplasm. Thus, lipid nanoparticle or microparticle encapsulation of one or more gRNAs and polynucleotide encoding Cas9 can be used to efficiently deliver both components to the cell. The Cas9 polynucleotide is then translated into Cas9 protein and can form a complex with the gRNA, which have also been transported into the cell by the same or different lipid nanoparticle or microparticle. In some embodiments, the polynucleotide encoding the Cas9 protein comprises a nuclear localization signal which promotes translocation of the Cas9 protein/gRNA complex to the nucleus. Alternatively, the at least one gRNA may cross the nuclear pore complex and form complexes with Cas9 protein in the nucleus. Once in the nucleus the gRNA/Cas9 complex scans the genome for homologous target sites and generates double strand breaks preferentially at the desired target site in the genome.

In various embodiments the lipid nanoparticles or microparticles are solid lipid nanoparticles (SLN), nanostructured lipid carriers (NLC), lipid-drug conjugate (LDC) nanoparticles, lipid nanocapsules (LNC), polymer lipid hybrid nanoparticles (PLN), or solid lipid microparticles (SLM).

According to various embodiments of the DNA targeting system, the gRNA(s) and polynucleotide of the invention can be encapsulated into the same lipid nanoparticle or microparticle, or the gRNA(s) and polynucleotide can each be encapsulated in separate lipid nanoparticles or microparticles. Thus, the DNA targeting system can comprise one or more lipid nanoparticles or microparticles for delivery of the nucleic acids of the invention.

In various embodiments, the lipid nanoparticles or microparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, nucleic acids, when present in the lipid nanoparticles, are resistant in aqueous solution to degradation with a nuclease.

In some embodiments, the lipid nanoparticles or microparticles comprise a cationic lipid. As used herein, the term “cationic lipid” refers to a lipid that is cationic or becomes cationic (protonated) as the pH is lowered below the pK of the ionizable group of the lipid, but it is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In certain embodiments, the cationic lipid comprises any of a number of lipid species that carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3dioleoyloxy)propyl)-N,N,N-trimethylammonium chloride (DOTAP); 3-(N-(N′,N′dimethylaminoethane)-carbamoyl)cholesterol (DC-Chol), N-(1-(2,3-dioleoyloxy)propyl)N-2-(sperminecarboxamido)ethyl)-N,N-dimethyl-ammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2dimyristyloxyprop-3-yl)-N,N-dimethyl-N-hydroxyethyl ammonium bromide (DMRIE).

Additionally, a number of commercial preparations of cationic lipids are available which can be used in the present invention. These include, for example, LIPOFECTIN (commercially available cationic liposomes comprising DOTMA and 1,2-dioleoyl-sn-3phosphoethanolamine (DOPE), from GIBCO/BRL, Grand Island, N.Y.); LIPOFECTAMINE (commercially available cationic liposomes comprising N-(1-(2,3dioleyloxy)propyl)-N-(2-(sperminecarboxamido)ethyl)-N,N-dimethyl-ammonium trifluoroacetate (DOSPA) and (DOPE), from GIBCO/BRL); and TRANSFECTAM (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In some embodiments, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

### 7. DNA Targeting Compositions

The present invention is also directed to DNA targeting compositions that comprise such DNA targeting systems. The DNA targeting compositions include at least one gRNA that targets a gene of interest. The gene of interest may be the dystrophin gene (e.g., human dystrophin gene), as described above, for example. The at least one gRNA molecule can bind and recognize a target region. The target regions can be chosen immediately upstream of possible out-of-frame stop codons such that insertions or deletions during the repair process restore the dystrophin reading frame by frame conversion. Target regions can also be splice acceptor sites or splice donor sites, such that insertions or deletions during the repair process disrupt splicing and restore the dystrophin reading frame by splice site disruption and exon exclusion. Target regions can also be aberrant stop codons such that insertions or deletions during the repair process restore the dystrophin reading frame by eliminating or disrupting the stop codon.

In certain embodiments, the presently disclosed composition comprising the DNA targeting system includes a first gRNA and a second gRNA, wherein the first gRNA molecule and the second gRNA molecule comprise a targeting domain that comprises a nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 110, SEQ ID NO: 111, or a complement thereof. In certain embodiments, the first gRNA molecule and the second gRNA molecule comprise different targeting domains. In certain embodiments, the first gRNA molecule comprises a nucleic acid sequence of SEQ ID NO: 1, SEQ ID NO: 3, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 110 and the second gRNA molecule comprises a nucleic acid sequence of SEQ ID NO: 2, SEQ ID NO: 4, SEQ ID NO: 5, SEQ ID NO: 6, SEQ ID NO: 16, SEQ ID NO: 17, SEQ ID NO: 18, SEQ ID NO: 19 or SEQ ID NO: 111. In certain embodiments, the first gRNA molecule is selected from the group consisting of SEQ ID NO: 1, SEQ ID NO: 11, SEQ ID NO: 12, SEQ ID NO: 13. SEQ ID NO: 14, SEQ ID NO: 15, and SEQ ID NO: 110 and the second gRNA molecule is selected from the group consisting of SEQ ID NO: 2, SEQ ID NO: 4, SEQ ID NO: 16. SEQ ID NO: 17, SEQ ID NO: 18, SEQ ID NO: 19, and SEQ ID NO: 111. In some embodiments, the gRNA is encoded by a polynucleotide selected from SEQ ID NOs: 1-19, 37-38, 41-42, 83-84, 110-111, or a fragment or a complement thereof. In some embodiments, the gRNA comprises a polynucleotide selected from SEQ ID NOs: 112-134, or a fragment or a complement thereof. In some embodiments, the first gRNA is encoded by a polynucleotide selected from SEQ ID NOs: 1, 3, 7-15, 37, 41, 83, and 110, or a fragment or a complement thereof, and the second gRNA is encoded by a polynucleotide selected from SEQ ID NOs: 2, 4-8, 16-19, 38, 42, 84, and 111, or a fragment or a complement thereof. In some embodiments, the first gRNA comprises a polynucleotide selected from SEQ ID NOs: 112-124, or a fragment or a complement thereof, and the second gRNA comprises a polynucleotide selected from SEQ ID NOs: 125-134, or a fragment or a complement thereof.

In certain embodiments, the first gRNA molecule and the second gRNA molecule are selected from the group consisting of: (i) a first gRNA molecule comprising a targeting domain that comprises a nucleotide sequence set forth in SEQ ID NO: 1, and a second gRNA molecule comprising a targeting domain that comprises a nucleotide sequence set forth in SEQ ID NO: 2; (ii) a first gRNA molecule comprising a targeting domain that comprises a nucleotide sequence set forth in SEQ ID NO: 11, and a second gRNA molecule comprising a targeting domain that comprises a nucleotide sequence set forth in SEQ ID NO: 4; (iii) a first gRNA molecule comprising a targeting domain that comprises a nucleotide sequence set forth in SEQ ID NO: 15, and a second gRNA molecule comprising a targeting domain that comprises a nucleotide sequence set forth in SEQ ID NO: 19; (iv) a first gRNA molecule comprising a targeting domain that comprises a nucleotide sequence set forth in SEQ ID NO: 15, and a second gRNA molecule comprising a targeting domain that comprises a nucleotide sequence set forth in SEQ ID NO: 18; (v) a first gRNA molecule comprising a targeting domain that comprises a nucleotide sequence set forth in SEQ ID NO: 15, and a second gRNA molecule comprising a targeting domain that comprises a nucleotide sequence set forth in SEQ ID NO: 4; (vi) a first gRNA molecule comprising a targeting domain that comprises a nucleotide sequence set forth in SEQ ID NO: 14, and a second gRNA molecule comprising a targeting domain that comprises a nucleotide sequence set forth in SEQ ID NO: 19; (vii) a first gRNA molecule comprising a targeting domain that comprises a nucleotide sequence set forth in SEQ ID NO: 14, and a second gRNA molecule comprising a targeting domain that comprises a nucleotide sequence set forth in SEQ ID NO: 18; (viii) a first gRNA molecule comprising a targeting domain that comprises a nucleotide sequence set forth in SEQ ID NO: 14, and a second gRNA molecule comprising a targeting domain that comprises a nucleotide sequence set forth in SEQ ID NO: 4; (ix) a first gRNA molecule comprising a targeting domain that comprises a nucleotide sequence set forth in SEQ ID NO: 11, and a second gRNA molecule comprising a targeting domain that comprises a nucleotide sequence set forth in SEQ ID NO: 19; (x) a first gRNA molecule comprising a targeting domain that comprises a nucleotide sequence set forth in SEQ ID NO: 14, and a second gRNA molecule comprising a targeting domain that comprises a nucleotide sequence set forth in SEQ ID NO: 15; (xi) a first gRNA molecule comprising a targeting domain that comprises a nucleotide sequence set forth in SEQ ID NO: 11, and a second gRNA molecule comprising a targeting domain that comprises a nucleotide sequence set forth in SEQ ID NO: 18; (xii) a first gRNA molecule comprising a targeting domain that comprises a nucleotide sequence set forth in SEQ ID NO: 41, and a second gRNA molecule comprising a targeting domain that comprises a nucleotide sequence set forth in SEQ ID NO: 42; and (xiii) a first gRNA molecule comprising a targeting domain that comprises a nucleotide sequence set forth in SEQ ID NO: 110, and a second gRNA molecule comprising a targeting domain that comprises a nucleotide sequence set forth in SEQ ID NO: 111. In some embodiments, the DNA targeting composition includes a nucleotide sequence set forth in SEQ ID NO: 37 and/or a nucleotide sequence set forth in SEQ ID NO: 38.

In certain embodiments, the DNA targeting composition may further include at least one Cas9 molecule or a Cas9 fusion protein that recognizes a PAM of either NNGRRT (SEQ ID NO: 24) or NNGRRV (SEQ ID NO: 25), or a polynucleotide encoding the Cas9 molecule or Cas9 fusion protein. In some embodiments, the DNA targeting composition includes a nucleotide sequence set forth in SEQ ID NO: 83 or SEQ ID NO: 84. In certain embodiments, the DNA targeting system is configured to form a first and a second double strand break in a first and a second intron flanking exon 51 of the human dystrophin gene, respectively, thereby deleting a segment of the dystrophin gene comprising exon 51.

The deletion efficiency of the presently disclosed DNA targeting system can be related to the deletion size, i.e., the size of the segment deleted by the DNA targeting system. In certain embodiments, the length or size of specific deletions is determined by the distance between the PAM sequences in the gene being targeted (e.g., a dystrophin gene). In certain embodiments, a specific deletion of a segment of the dystrophin gene, which is defined in terms of its length and a sequence it comprises (e.g., exon 51), is the result of breaks made adjacent to specific PAM sequences within the target gene (e.g., a dystrophin gene).

In certain embodiments, the deletion size is about 50 to about 2,000 base pairs (bp), e.g., about 50 to about 1999 bp, about 50 to about 1900 bp, about 50 to about 1800 bp, about 50 to about 1700 bp, about 50 to about 1650 bp, about 50 to about 1600 bp, about 50 to about 1500 bp, about 50 to about 1400 bp, about 50 to about 1300 bp, about 50 to about 1200 bp, about 50 to about 1150 bp, about 50 to about 1100 bp, about 50 to about 1000 bp, about 50 to about 900 bp, about 50 to about 850 bp, about 50 to about 800 bp, about 50 to about 750 bp, about 50 to about 700 bp, about 50 to about 600 bp, about 50 to about 500 bp, about 50 to about 400 bp, about 50 to about 350 bp, about 50 to about 300 bp, about 50 to about 250 bp, about 50 to about 200 bp, about 50 to about 150 bp, about 50 to about 100 bp, about 100 to about 1999 bp, about 100 to about 1900 bp, about 100 to about 1800 bp, about 100 to about 1700 bp, about 100 to about 1650 bp, about 100 to about 1600 bp, about 100 to about 1500 bp, about 100 to about 1400 bp, about 100 to about 1300 bp, about 100 to about 1200 bp, about 100 to about 1150 bp, about 100 to about 1100 bp, about 100 to about 1000 bp, about 100 to about 900 bp, about 100 to about 850 bp, about 100 to about 800 bp, about 100 to about 750 bp, about 100 to about 700 bp, about 100 to about 600 bp, about 100 to about 1000 bp, about 100 to about 400 bp, about 100 to about 350 bp, about 100 to about 300 bp, about 100 to about 250 bp, about 100 to about 200 bp, about 100 to about 150 bp, about 200 to about 1999 bp, about 200 to about 1900 bp, about 200 to about 1800 bp, about 200 to about 1700 bp, about 200 to about 1650 bp, about 200 to about 1600 bp, about 200 to about 1500 bp, about 200 to about 1400 bp, about 200 to about 1300 bp, about 200 to about 1200 bp, about 200 to about 1150 bp, about 200 to about 1100 bp, about 200 to about 1000 bp, about 200 to about 900 bp, about 200 to about 850 bp, about 200 to about 800 bp, about 200 to about 750 bp, about 200 to about 700 bp, about 200 to about 600 bp, about 200 to about 2000 bp, about 200 to about 400 bp, about 200 to about 350 bp, about 200 to about 300 bp, about 200 to about 250 bp, about 300 to about 1999 bp, about 300 to about 1900 bp, about 300 to about 1800 bp, about 300 to about 1700 bp, about 300 to about 1650 bp, about 300 to about 1600 bp, about 300 to about 1500 bp, about 300 to about 1400 bp, about 300 to about 1300 bp, about 300 to about 1200 bp, about 300 to about 1150 bp, about 300 to about 1100 bp, about 300 to about 1000 bp, about 300 to about 900 bp, about 300 to about 850 bp, about 300 to about 800 bp, about 300 to about 750 bp, about 300 to about 700 bp, about 300 to about 600 bp, about 300 to about 3000 bp, about 300 to about 400 bp, or about 300 to about 350 bp. In certain embodiments, the deletion size can be about 118 base pairs, about 233 base pairs, about 326 base pairs, about 766 base pairs, about 805 base pairs, or about 1611 base pairs.

The gRNA may target a nucleotide sequence selected from the group consisting of SEQ ID NO: 1-19, 41, 42, 37, 38, 41, 42, 83, 84, 110, and 111, or a complement thereof or a fragment thereof. For example, the disclosed DNA targeting systems may be engineered to mediate highly efficient gene editing at exon 51 of the dystrophin gene. These DNA targeting systems may restore dystrophin protein expression in cells from DMD patients. In various embodiments, the DNA targeting systems composition includes a nucleotide sequence set forth in SEQ ID NO: 110, a nucleotide sequence set forth in SEQ ID NO: 111, a nucleotide sequence set forth in SEQ ID NO: 37, a nucleotide sequence set forth in SEQ ID NO: 38, a nucleotide sequence set forth in SEQ ID NO: 83, and/or a nucleotide sequence set forth in SEQ ID NO: 84.

### 8. Methods

a. Methods of Correcting a Mutant Gene and Treating a Subject

The presently disclosed subject matter provides for methods of correcting a mutant gene (e.g., a mutant dystrophin gene, e.g., a mutant human dystrophin gene) in a cell and treating a subject suffering from a genetic disease, such as DMD. The method can include administering to a cell or a subject a presently disclosed lipid nanoparticle or microparticle, DNA targeting system, or a composition comprising thereof as described above. The method can comprise administering to the subject the presently disclosed lipid nanoparticle or microparticle, DNA targeting system, or a composition comprising thereof for genome editing, as described above. Use of presently disclosed lipid nanoparticle or microparticle to deliver the DNA targeting system comprising the at least one gRNA and the polynucleotide to the subject may restore the expression of a full-functional or partially-functional protein with a repair template or donor DNA, which can replace the entire gene or the region containing the mutation. The DNA targeting system may be used to introduce site-specific double strand breaks at targeted genomic loci. Site-specific double-strand breaks may be created when the DNA targeting system binds to a target DNA sequence, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

The present disclosure is also directed to genome editing with a DNA targeting system without a repair template, which may efficiently correct the reading frame and restore the expression of a functional protein involved in a genetic disease. The disclosed DNA targeting system may involve using homology-directed repair or nuclease-mediated non-homologous end joining (NHEJ)-based correction approaches, which enable efficient correction in proliferation-limited primary cell lines that may not be amenable to homologous recombination or selection-based gene correction. This strategy integrates the rapid and robust assembly of active DNA targeting systems with an efficient gene editing method for the treatment of genetic diseases caused by mutations in nonessential coding regions that cause frameshifts, premature stop codons, aberrant splice donor sites or aberrant splice acceptor sites.

i. Nuclease Mediated Non-Homologous End Joining

Restoration of protein expression from an endogenous mutated gene may be through template-free NHEJ-mediated DNA repair. In contrast to a transient method targeting the target gene RNA, the correction of the target gene reading frame in the genome by a transiently expressed DNA targeting system may lead to permanently restored target gene expression by each modified cell and all of its progeny. In certain embodiments, NHEJ is a nuclease mediated NHEJ, which in certain embodiments, refers to NHEJ that is initiated by a Cas9 molecule, cuts double stranded DNA. The method comprises administering a presently disclosed DNA targeting system or a composition comprising thereof to the subject in need.

Nuclease mediated NHEJ gene correction may correct the mutated target gene and offers several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment. NHEJ-based gene correction using a DNA targeting system, as well as other engineered nucleases including meganucleases and zinc finger nucleases, may be combined with other existing ex vivo and in vivo platforms for cell- and gene-based therapies, in addition to the lipid nanoparticle approach described here. For example, delivery of a DNA targeting system by mRNA-based gene transfer or as purified cell permeable proteins could enable a DNA-free genome editing approach that would circumvent any possibility of insertional mutagenesis.

ii. Homology-Directed Repair

Restoration of protein expression from an endogenous mutated gene may involve homology-directed repair. The method as described above further includes administrating a donor template to the cell. The donor template may include a nucleotide sequence encoding a full-functional protein or a partially-functional protein. For example, the donor template may include a miniaturized dystrophin construct, termed minidystrophin (“minidys”), a full-functional dystrophin construct for restoring a mutant dystrophin gene, or a fragment of the dystrophin gene that after homology-directed repair leads to restoration of the mutant dystrophin gene.

b. Methods of Treating Disease

The present disclosure is directed to a method of treating a subject in need thereof. The method comprises administering to a tissue or cell of a subject the presently disclosed lipid nanoparticle or microparticle, DNA targeting system, or a composition comprising thereof, as described above. In certain embodiments, the method may comprise administering to the skeletal muscle tissue or cell, smooth muscle tissue or cell, or cardiac muscle tissue or cell of the subject the presently disclosed lipid nanoparticle or microparticle, DNA targeting system, or composition comprising thereof, as described above. In certain embodiments, the method may comprise administering to a vein of the subject the presently disclosed lipid nanoparticle or microparticle, DNA targeting system, or composition comprising thereof, as described above. In certain embodiments, the subject is suffering from a skeletal muscle or cardiac muscle condition causing degeneration or weakness or a genetic disease. For example, the subject may be suffering from Duchenne muscular dystrophy, as described above.

The method, as described above, may be used for correcting the dystrophin gene and recovering full-functional or partially-functional protein expression of said mutated dystrophin gene. In various aspects of the invention, dystrophin expression in the subject is increased by at least 1%, 2%, 5%, 10%, 15%, 20%, 25%, 30%, 35%, 40%, 45%, or at least 50%.

In some aspects and embodiments, the disclosure provides a method for reducing the effects (e.g., clinical symptoms/indications) of DMD in a patient. In some aspects and embodiments, the disclosure provides a method for treating DMD in a patient. In some aspects and embodiments, the disclosure provides a method for preventing DMD in a patient. In some aspects and embodiments, the disclosure provides a method for preventing further progression of DMD in a patient.

### 9. Pharmaceutical Compositions

The presently disclosed subject matter provides for a composition comprising the above-described lipid nanoparticle or microparticle or DNA targeting system. The composition may be a pharmaceutical composition. The pharmaceutical compositions according to the present invention can be formulated according to the mode of administration to be used. In cases where pharmaceutical compositions are injectable pharmaceutical compositions, they may be sterile, pyrogen free and particulate free. An isotonic formulation may preferably be used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents.

### 10. Routes of Administration

The presently disclosed DNA targeting system or a composition comprising thereof may be administered to a subject by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. In certain embodiments, the presently disclosed DNA targeting system or a composition is administered to a subject (e.g., a subject suffering from DMD) intramuscularly, intravenously or a combination thereof. For veterinary use, the presently disclosed DNA targeting system or compositions may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gone guns”, or other physical methods such as electroporation (“EP”), “hydrodynamic method”, or ultrasound.

In some embodiments, the presently disclosed DNA targeting system or a composition thereof is administered by 1) tail vein injections (systemic) into adult mice; 2) intramuscular injections, for example, local injection into a muscle such as the TA or gastrocnemius in adult mice; 3) intraperitoneal injections into P2 mice; or 4) facial vein injection (systemic) into P2 mice.

The presently disclosed DNA targeting system can be administered to a subject at any time during the life cycle, including after maturity and at any time during development. In various embodiments, the DNA targeting system and compositions comprising thereof can be administered to the subject before birth or within 1-2 days of birth.

### 11. Cell Types

Any of these delivery methods and/or routes of administration can be utilized with a myriad of cell types, for example, those cell types currently under investigation for cell-based therapies of DMD, including, but not limited to, immortalized myoblast cells, such as wild-type and DMD patient derived lines, for example Δ48-50 DMD, DMD 6594 (del48-50), DMD 8036 (del48-50), C25C14 and DMD-7796 cell lines, primal DMD dermal fibroblasts, induced pluripotent stem cells, bone marrow-derived progenitors, skeletal muscle progenitors, human skeletal myoblasts from DMD patients, CD 133.sup.+ cells, mesoangioblasts, cardiomyocytes, hepatocytes, chondrocytes, mesenchymal progenitor cells, hematopoetic stem cells, smooth muscle cells, and MyoD- or Pax7-transduced cells, or other myogenic progenitor cells. Immortalization of human myogenic cells can be used for clonal derivation of genetically corrected myogenic cells. Cells can be modified ex vivo to isolate and expand clonal populations of immortalized DMD myoblasts that include a genetically corrected dystrophin gene and are free of other nuclease-introduced mutations in protein coding regions of the genome. In vivo delivery of the gRNAs and mRNA of the DNA targeting system by non-viral gene transfer using the lipid nanoparticles or microparticles of the system, may enable highly specific correction in situ with minimal or no risk of exogenous DNA integration. In some embodiments, the lipid nanoparticle or microparticle as detailed herein is for delivering a DNA targeting system to a muscle cell. The muscle cell may be a skeletal muscle cell, a cardiac muscle cell, and/or a smooth muscle cell.

### 12. Kits

Provided herein is a kit, which may be used to correct a mutated dystrophin gene. The kit comprises the lipid nanoparticle or microparticle or DNA targeting system or a composition comprising thereof, for correcting a mutated dystrophin gene and instructions for using the system or composition.

Instructions included in kits may be affixed to packaging material or may be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The DNA targeting system or a composition comprising thereof for correcting a mutated dystrophin or genome editing of a dystrophin gene in a subject will include the lipid nanoparticles or microparticles that encapsulate the gRNA molecules and a mRNA encoding the Cas9 molecule, as described above, that specifically binds and cleaves a region of the dystrophin gene. The DNA targeting system, as described above, may be included in the kit to specifically bind and target a particular region in the mutated dystrophin gene. The kit may further include donor DNA, a different gRNA, or a transgene, as described above.

### 13. Examples

### Example 1

### Non-Viral, Lipid Nanoparticle Delivery of CRISPR for Treating DMD Mutations

Using INVIVOFECTAMINE, formulations of mRNA/gRNA or RNPs were injected into mice. INVIVOFECTAMINE was optimized for the following nucleotide delivery: (1) SpCas9 mRNA s modified with N and C terminal NLS HA tag and modified uridine substitution (0.5 mg/kg) and (2) SpCas9 gRNAs (0.25 mg/kg each) with Phosphorothioated 2′ O-methyl bases—3 bp on each end. SaCas9 (0.25 mg/kg) was also delivered to the mice.

The sequences of the target sequences of the SpCas9 gRNAs were SEQ ID NO: 110 (GATTGGCTTTGATTTCCCTA) and SEQ ID NO: 111 (GCAGTTGCCTAAGAACTGGT).

INVIVOFECTAMINE preparation. INVIVOFECTAMINE (24 uL) was prepared according to the manufacturer's instructions with mRNA or RNP: 100 μL of a 1.2-mg/mL siRNA solution was prepared by mixing the following components in a 1:1 ratio: 50 μL of siRNA duplex solution (2.4-mg/mL) and 50 μl of complexation buffer. INVIVOFECTAMINE reagent was brought to room temperature and 100 μL was added to a 1.5-mL tube. Diluted siRNA solution was immediately added to INVIVOFECTAMINE reagent in the tube. The tube was vortexed immediately to ensure INVIVOFECTAMINE-siRNA complexation. The INVIVOFECTAMINE-siRNA duplex mixture was incubated for 30 minutes at 50° C. The tube was briefly centrifuged to collect the sample. The complex was diluted 6-fold by adding 1 mL of PBS pH7.4 and mixed well. The INVIVOFECTAMINE-siRNA was then ready for in vivo delivery.

Intramuscular injections of AAV. 7-8 week old male hDMDΔ52/mdx mice were anesthetized and placed on a warming pad. hDMDΔ52/mdx mice are mdx mice carrying the human dystrophin gene, but are engineered to be missing exon 52. Thus, the mouse model mimics the human DMD mutation and is correctable by exon 51 skipping. The tibialis anterior (TA) muscle was prepared for injection of 24 μl of INVIVOFECTAMINE with mRNA or RNP, or saline into the right or left TA, respectively. After 4 weeks mice were euthanized via CO₂ inhalation and tissues were collected into RNALater (Life Technologies) for analysis.

Male hDMD/d52 mice were injected with mRNA/gRNA or RNPs into the TA or TV and harvested 4 weeks post injection. Genomic DNA was extracted from the treated muscle and PCR across the region revealed the intended deletion.

ELISA for Humoral Response. Antibodies against SpCas9 or SaCas9 were detected by adapting a protocol from Wang et al., and Chew et al. Recombinant SpCas9 or SaCas9 protein was diluted in 1× coating buffer (KPL) and used to coat a 96-well Nunc MaxiSorp plate with 0.5 μg of protein per well. Protein was incubated overnight at 4° C. to adsorb to the plate. Plates were washed three times for 5 minutes each with 1× wash buffer (KPL). Plates were blocked with 1% BSA blocking solution (KPL) for 1 hour at room temperature. Standard curves for IgG were generated using an αSpCas9 or αSaCas9 antibody (Diagenode C15200230). Serum samples were added in dilutions ranging from 1:40 to 1:20000 and plates were incubated for 5 hrs at 4° C. with shaking. Plates were washed 3 times for 5 minutes each and 100 μL of blocking solution containing goat-anti mouse IgG (Sigma 1:4000) was added to each well and incubated at 1 hr at room temperature. Plates were washed 4 times for 5 minutes each and 100 μL of ABTS ELISA HRP substrate (KPL) was added to each well. Optical density (OD) at 410 nm was measured with a plate reader.

PCR of Genomic DNA to Monitor Genome Editing-Genomic DNA analysis. Mouse tissues were digested in Buffer ALT and proteinase K at 56° C. in a shaking heat block. Cells were digested in Buffer AL and proteinase K at 56° C. for 10 minutes. DNEasy kit (Qiagen) was used to collect genomic DNA. Nested endpoint PCR was performed with primers flanking the SaCas9/gRNA cut sites in the intronic regions using AccuPrime High Fidelity PCR kit. PCR products were electrophoresed in a 1% agarose gel and viewed on a BioRad GelDoc imager to observe the parent band and deletion product. The deletion product was sequenced by first purification of the sample using the QIAQuick Gel Extraction kit (Qiagen) then Sanger sequencing (Eton Bioscience).

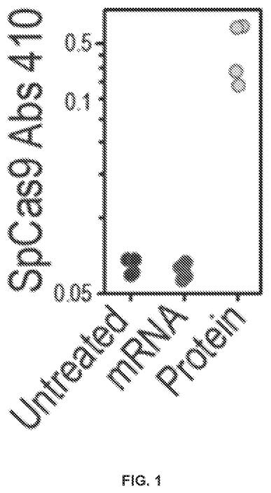

RNPs elicited a strong humoral response and mRNA did not. FIG. 1 shows ELISA against SpCas9 shows humoral response against SpCas9 after injection of RNPs but not mRNA-encoded SpCas9.

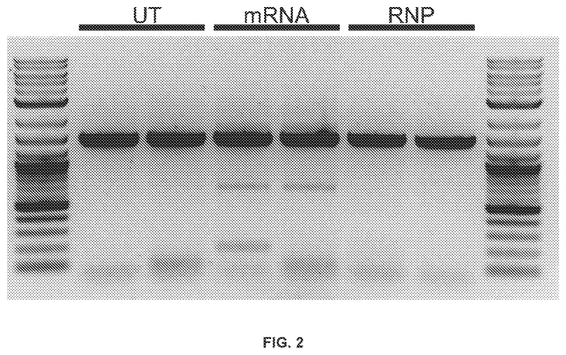

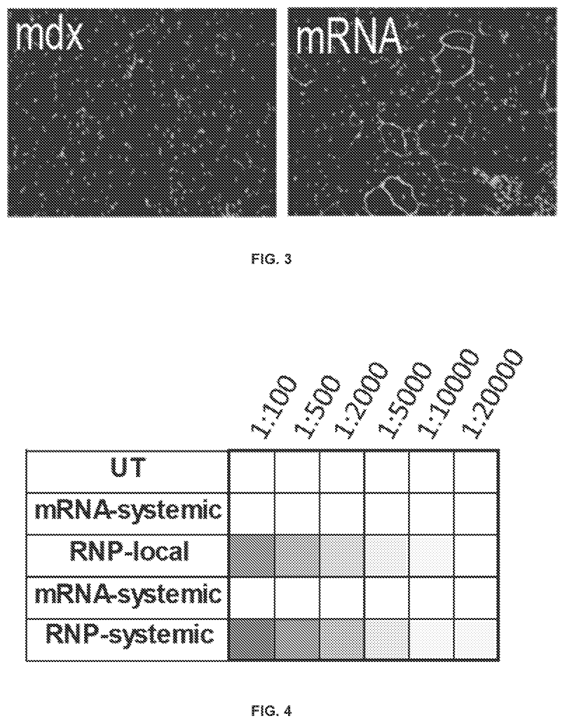

mRNA was able to edit the target gene while RNP was not. FIG. 2 shows that mRNA was able to delete exon 51 from hDMD/d52 mice and FIG. 3 shows that the mRNA deletion of exon 51 restores dystrophin.

FIG. 4 shows that mRNA injection does not lead to a humoral response. RNP administration raises antibodies in local or systemic injections.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A lipid nanoparticle or microparticle for delivering a DNA targeting system to a muscle cell, the DNA targeting system comprising: at least one gRNA molecule targeting a fragment of a mutant dystrophin gene; and/or a polynucleotide encoding a Cas9 nuclease.

Clause 2. The lipid nanoparticle or microparticle of clause 1, wherein the at least one gRNA molecule comprises a first gRNA molecule and a second gRNA molecule.

Clause 3. The lipid nanoparticle or microparticle of clause 1 or 2, wherein the polynucleotide encoding a Cas9 nuclease is mRNA.

Clause 4. The lipid nanoparticle or microparticle of any one of clauses 1-3, wherein the first gRNA molecule and the second gRNA molecule each comprise a targeting domain, wherein the first gRNA molecule is encoded by a polynucleotide comprising a nucleotide sequence selected from SEQ ID NO: 1, SEQ ID NO: 3, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 37, SEQ ID NO: 41, SEQ ID NO: 83, or SEQ ID NO: 110 or a fragment or complement thereof or comprises a nucleotide sequence selected from SEQ ID NOs: 112-124 or a fragment or complement thereof, wherein the second gRNA molecule is encoded by a polynucleotide comprising a nucleotide sequence selected from SEQ ID NO: 2, SEQ ID NO: 4, SEQ ID NO: 5, SEQ ID NO: 6, SEQ ID NO: 16, SEQ ID NO: 17, SEQ ID NO: 18, SEQ ID NO: 19, SEQ ID NO: 38, SEQ ID NO: 42, SEQ ID NO: 84, or SEQ ID NO: 111 or a fragment or complement thereof or comprises a nucleotide sequence selected from SEQ ID NOs: 125-134 or a fragment or complement thereof, and wherein the first gRNA molecule and the second gRNA molecule comprise different targeting domains.

Clause 5. The lipid nanoparticle or microparticle of clause 4, wherein the first gRNA molecule comprises a targeting domain comprising the nucleotide sequence of SEQ ID NO: 110 or a fragment or complement thereof or comprises the nucleotide sequence of SEQ ID NO: 124 or a fragment or complement thereof, and wherein the second gRNA molecule comprises a targeting domain comprising the nucleotide sequence of SEQ ID NO: 111 or a fragment or complement thereof or comprises the nucleotide sequence of SEQ ID NO: 134 or a fragment or complement thereof.

Clause 6. The lipid nanoparticle or microparticle of any one of clauses 1-5, wherein the at least one gRNA and the polynucleotide encoding the Cas9 nuclease are encapsulated in the same lipid nanoparticle or microparticle.

Clause 7. The lipid nanoparticle or microparticle of clause any one of clauses 1-6, wherein the at least one gRNA and the polynucleotide encoding the Cas9 nuclease are each encapsulated in a separate lipid nanoparticle.

Clause 8. The lipid nanoparticle or microparticle of any one of clauses 1-7, wherein the lipid nanoparticle or microparticle is selected from the group consisting of solid lipid nanoparticle (SLN), nanostructured lipid carrier (NLC), lipid-drug conjugate (LDC) nanoparticle, lipid nanocapsule (LNC), polymer lipid hybrid nanoparticle (PLN), and solid lipid microparticle (SLM).

Clause 9. The lipid nanoparticle or microparticle of clause 8, wherein the lipid nanoparticle or microparticle is a solid lipid nanoparticle (SLN).

Clause 10. The lipid nanoparticle or microparticle of clause 8, wherein the lipid nanoparticle or microparticle is a nanostructured lipid carrier (NLC).

Clause 11. The lipid nanoparticle or microparticle of clause 8, wherein the lipid nanoparticle or microparticle is a lipid-drug conjugate (LDC) nanoparticle.

Clause 12. The lipid nanoparticle or microparticle of clause 8, wherein the lipid nanoparticle or microparticle is a lipid nanocapsule (LNC).

Clause 13. The lipid nanoparticle or microparticle of clause 8, wherein the lipid nanoparticle or microparticle is a polymer lipid hybrid nanoparticle (PLN).

Clause 14. The lipid nanoparticle or microparticle of clause 8, wherein the lipid nanoparticle or microparticle is a solid lipid microparticle (SLM).

Clause 15. The lipid nanoparticle or microparticle of any one of clauses 1-14, wherein the at least one gRNA molecule targets an exon selected from exons 1-8, 10, 11, 12, 14, 16-22, 43-59, and 61-66 of the mutant dystrophin gene, or an intron that flanks an exon selected from exons 1-8, 10, 11, 12, 14, 16-22, 43-59, and 61-66 of the mutant dystrophin gene.

Clause 16. The lipid nanoparticle or microparticle of any one of clauses 1-15, wherein the DNA targeting system further comprises a donor sequence that comprises an exon of the wild-type dystrophin gene or a functional equivalent thereof, and wherein the exon is selected from exons 1-8, 10, 11, 12, 14, 16-22, 43-59, and 61-66 of the wild-type dystrophin gene.

Clause 17. The lipid nanoparticle or microparticle of any one of clauses 1-16, wherein the at least one gRNA molecule targets two introns that flank exon 51 of a human dystrophin gene.

Clause 18. The lipid nanoparticle or microparticle of any one of clauses 1-17, wherein the DNA targeting system induces a first double strand break in a first intron flanking exon 51 of a human dystrophin gene and a second double strand break in a second intron flanking exon 51 of a human dystrophin gene.

Clause 19. The lipid nanoparticle or microparticle of any one of clauses 1-18, wherein the polynucleotide encodes SpCas9 or SaCas9.

Clause 20. The lipid nanoparticle or microparticle of any one of clauses 3-19, wherein the mRNA is a modified mRNA.

Clause 21. The lipid nanoparticle or microparticle of clause 20, wherein the modified mRNA comprises one or more modifications selected from an N terminal NLS, a C terminal NLS, an HA Tag, and a uridine substitution.

Clause 22. The lipid nanoparticle or microparticle of any one of clauses 1-21, wherein the muscle cell is selected from a skeletal muscle cell, a cardiac muscle cell, and a smooth muscle cell.

Clause 23. A composition comprising the lipid nanoparticle or microparticle of any one of clauses 1-22 and a pharmaceutically acceptable carrier.

Clause 24. A method of treating Duchenne Muscular Dystrophy in a subject, the method comprising administering to the subject the lipid nanoparticle or microparticle of any one of clauses 1-22 or the composition of clause 23.

Clause 25. The method of clause 24, wherein the subject experiences no or a limited humoral response that is cross reactive to the Cas9 nuclease after administration.

Clause 26. The method of clause 24 or 25, where the subject comprises a mutant dystrophin gene.

Clause 27. A method of genome editing a mutant dystrophin gene in a subject, the method comprising administering to the subject the lipid nanoparticle or microparticle of any one of clauses 1-22 or the composition of clause 23.

Clause 28. The method of any one of clauses 26-27, wherein the mutant dystrophin gene comprises a premature stop codon, a disrupted reading frame, an aberrant splice acceptor site, or an aberrant splice donor site, or a combination thereof.

Clause 29. The method of any one of clauses 26-27, wherein the mutant dystrophin gene comprises a frameshift mutation that causes a premature stop codon and a truncated gene product.

Clause 30. The method of any one of clauses 26-27, wherein the mutant dystrophin gene comprises a deletion of one or more exons that disrupts the reading frame.

Clause 31. The method of clause 27, wherein genome editing of the mutant dystrophin gene comprises a deletion of a premature stop codon, correction of a disrupted reading frame, modulation of splicing by disruption of a splice acceptor site, modulation of splicing by disruption of a splice donor sequence, deletion of exon 51, or a combination thereof.

Clause 32. The method of any one of clauses 27-31, wherein the mutant dystrophin gene is edited by homology-directed repair.

Clause 33. The method of any one of clauses 24-32, wherein dystrophin expression in the subject is increased by at least 1%, 2%, 5%, 10%, 15%, 20%, 25%, 30%, 35%, 40%, 45%, or at least 50% after editing.

Clause 34. The method of any one of clauses 24-33, wherein the lipid nanoparticle or microparticle is administered to the subject before birth or within 1-2 days of birth.

Clause 35. The method of any one of clauses 24-34, wherein the lipid nanoparticle or microparticle is administered to the subject intramuscularly, intravenously, or a combination thereof.

Clause 36. The method of any one of clauses 24-35, wherein administration of the lipid nanoparticle or the microparticle or the compositions leads to expression of a functional or partially-functional dystrophin protein in the subject.

Clause 37. A kit comprising the lipid nanoparticle or microparticle of any one of clauses 1-22.

SEQUENCES

| | | |
|---|---|---|
| SEQ ID NO: 1 | JCR89 | aaaGATATATAATGTCATGAAT |
| SEQ ID NO: 2 | JCR91 | gcaGAATCAAATATAATAGTCT |
| SEQ ID NO: 3 | JCR159 | CAATTAAATTTGACTTATTGTT |
| SEQ ID NO: 4 | JCR160 | CTAGACCATTTCCCACCAGTTC |
| SEQ ID NO: 5 | JCR167 | AGGACTTTTATTTACCAAAGGA |
| SEQ ID NO: 6 | JCR166 | ATCCAAGTCCATTTGATTCCTA |
| SEQ ID NO: 7 | JCR168 | TAATTCTTTCTAGAAAGAGCCT |
| SEQ ID NO: 8 | JCR170 | GGACATGTGCAAGATGCAAGAG |
| SEQ ID NO: 9 | JCR171 | TGTATGTAGAAGACCTCTAAGT |
| SEQ ID NO: 10 | JCR156 | TCCCCTCACCACTCACCTCTGA |
| SEQ ID NO: 11 | JCR157 | CTCTGATAAGCAGCTGTGTGt |
| SEQ ID NO: 12 | JCR176 | ctCtgataACCCAGctgtg |
| SEQ ID NO: 13 | JCR177 | ctctgataACCCAGctgtgt |
| SEQ ID NO: 14 | JCR178 | ctctgataACCCAGctgtgtg |
| SEQ ID NO: 15 | JCR179 | ctctgataACCCAGctgtgtgtt |
| SEQ ID NO: 16 | JCR180 | ctagaccatttcccaccag |
| SEQ ID NO: 17 | JCR181 | ctagaccatttcccaccagt |
| SEQ ID NO: 18 | JCR182 | ctagaccatttcccaccagtt |
| SEQ ID NO: 19 | JCR183 | ctagacaatttcccaccagttct |
| SEQ ID NO: 20 | recognition sequence | NNAGAAW (W = A or T) |
| SEQ ID NO: 21 | recognition sequence | NAAR (R = A or G; |
| SEQ ID NO: 22 | recognition sequence | NNGRR (R = A or G) |
| SEQ ID NO: 23 | recognition sequence | NNGRRN (R - A or G) |
| SEQ ID NO: 24 | recognition sequence | NNGRRT (R = A or G) |
| SEQ ID NO: 25 | recognition sequence | NNGRRV (R = A or G) |

SEQ ID NO: 26
codon optimized nucleic acid sequence encoding a S. pyogenes Cas9 molecule
atggataaaaagtacagcatcgggctggacatcggtacaaactcagtggggtgggccgtgat
tacggacgagtacaaggtaccctccaaaaaatttaaagtgctgggtaacacggacagacact
ctataaagaaaaatcttattggagccttgctgttcgactcaggcgagacagccgaagccaca
aggttgaagcggaccgccaggaggcggtataccaggagaaagaaccgcatatgctacctgca
agaaatcttcagtaacgagatggcaaaggttgacgatagctttttccatcgcctggaagaat
cctttcttgttgaggaagacaagaagcacgaacggcaccccatctttggcaatattgtcgac
gaagtggcatatcacgaaaagtacccgactatctaccacctcaggaagaagctggtggactc
taccgataaggcggacctcagacttatttatttggcactcgcccacatgattaaatttagag
gacatttcttgatcgagggcgacctgaacccggacaacagtgacgtcgataagctgttcatc
caacttgtgcagacctacaatcaactgttcgaagaaaaccctataaatgcttcaggagtcga
cgctaaagcaatcctgtccgcgcgcctctcaaaatctagaagacttgagaatctgattgctc
agttgcccggggaaaagaaaaatggattgtttggcaacctgatcgccctcagtctcggactg
accccaaatttcaaaagtaacttcgacctggccgaagacgctaagctccagctgtccaagga
cacatacgatgacgacctcgacaatctgctggcccagattggggatcagtacgccgatctct
ttttggcagcaaagaacctgtccgacgccatcctgttgagcgatatcttgagagtgaacacc
gaaattactaaagcaccccttagcgcatctatgatcaagcggtacgacgagcatcatcagga
tctgaccctgctgaaggctcttgtgaggcaacagctccccgaaaaatacaaggaaatcttct
ttgaccagagcaaaaacggctacgctggctatatagatggtggggccagtcaggaggaattc
tataaattcatcaagcccattctcgagaaaatggacggcacagaggagttgctggtcaaact
taacagggaggacctgctgcggaagcagcggacctttgacaacgggtctatcccccaccaga
ttcatctgggcgaactgcacgcaatcctgaggaggcaggaggatttttatccttttcttaaa
gataaccgcgagaaaatagaaaagattcttacattcaggatcccgtactacgtgggacctct
cgcccggggcaattcacggtttgcctggatgacaaggaagtcagaggagactattacacctt
ggaacttcgaagaagtggtggacaagggtgcatctgcccagtctttcatcgagcggatgaca
aattttgacaagaacctccctaatgagaaggtgctgcccaaacattctctgctctacgagta
ctttaccgtctacaatgaactgactaaagtcaagtacgtcaccgagggaatgaggaagccgg
cattccttagtggagaacagaagaaggcgattgtagacctgttgttcaagaccaacaggaag
gtgactgtgaagcaacttaaagaagactactttaagaagatcgaatgttttgacagtgtgga
aatttcaggggttgaagaccgcttcaatgcgtcattggggacttaccatgatcttctcaaga
tcataaaggacaaagacttcctggacaacgaagaaaatgaggatattctcgaagacatcgtc
ctcaccctgaccctgttcgaagacagggaaatgatagaagagcgcttgaaaacctatgccca
cctcttcgacgataaagttatgaagcagctgaagcgcaggagatacacaggatggggaagat
tgtcaaggaagctgatcaatggaattagggataaacagagtggcaagaccatactggatttc
ctcaaatctgatggcttogccaataggaacttcatgcaactgattcacgatgactctcttac
cttcaaggaggacattcaaaaggctcaggtgagcgggcagggagactcccttcatgaacaca
tcgcgaatttggcaggttcccccgctattaaaaagggcatccttcaaactgtcaaggtggtg
gatgaattggtcaaggtaatgggcagacataagccagaaaatattgtgatcgagatggcccg
cgaaaaccagaccacacagaagggccagaaaaatagtagagagcggatgaagaggatcgagg
agggcatcaaagagctgggatctcagattctcaaagaacaccccgtagaaaacacacagctg
cagaacgaaaaattgtacttgtactatctgcagaacggcagagacatgtacgtcgaccaaga
acttgatattaatagactgtccgactatgacgtagaccatatcgtgccccagtccttcctga
aggacgactccattgataacaaagtcttgacaagaagcgacaagaacaggggtaaaagtgat
aatgtgcctagcgaggaggtggtgaaaaaaatgaagaactactggcgacagctgcttaatgc
aaagctcattacacaacggaagttcgataatctgacgaaagcagagagaggtggcttgtctg
agttggacaaggcagggtttattaagcggcagctggtggaaactaggcagatcacaaagcac
gtggogcagattttggacagccggatgaacacaaaatacgacgaaaatgataaactgatacg
agaggtcaaagttatcacgctgaaaagcaagctggtgtccgattttcggaaagacttccagt
tctacaaagttcgcgagattaataactaccatcatgctcacgatgcgtacctgaacgctgtt
gtcgggaccgccttgataaagaagtacccaaagctggaatccgagttcgtatacggggatta
caaagtgtacgatgtgaggaaaatgatagccaagtccgagcaggagattggaaaggccacag
ctaagtacttcttttattctaacatcatgaatttttttaagacggaaattaccctggccaac
ggagagatcagaaagcggccccttatagagacaaatggtgaaacaggtgaaatcgtctggga
taagggcagggatttcgctactgtgaggaaggtgctgagtatgccacaggtaaatatcgtga
aaaaaaccgaagtacagaccggaggattttccaaggaaagcattttgcctaaaagaaactca
gacaagctcatcgcccgcaagaaagattgggaccctaagaaatacgggggatttgactcacc
caccgtagcctattctgtgctggtggtagctaaggtggaaaaaggaaagtctaagaagctga
agtccgtgaaggaactcttgggaatcactatcatggaaagatcatcctttgaaaagaaccct
atcgatttcctggaggctaagggttacaaggaggtcaagaaagacctcatcattaaactgcc
aaaatactctctcttcgagctggaaaatggcaggaagagaatgttggccagcgccggagagc
tgcaaaagggaaacgagcLLyoLctgccctccaaatatgttaattttctctatctcgcttcc
cactatgaaaagctgaaagggtctcccgaagataacgagcagaagcagctgttcgtcgaaca
gcacaagcactatctggatgaaataatcgaacaaataagcgagttcagcaaaagggttatcc
tggcggatgctaatttggacaaagtactgtctgcttataacaagcaccgggataagcctatt
agggaacaagccgagaatataattcacctctttacactcacgaatctcggagcccccgccgc
cttcaaatactttgatacgactatcgaccggaaacggtataccagtaccaaagaggtcotcg
atgccaccctcatccaccagtcaattactggcctgtacgaaacacggatcgacctctctcaa
ctgggcggcgactag
SEQ ID NO: 27
corresponding amino acid sequence of an S. pyogenes Cas9 molecule
MDKKYSIGLDIGTNSVGWAVITDEYKVPSKKFKVLGNTDRHSIKKNLIGALLFDSGETAEAT
RLKRTAKRRYTRRKNRICYLQEIFSNEMAKVDDSFFHRLEESFLVEEDKKHERHPIFGNIVD
EVAYHEKYFTIYHLRKKLVDSTDKADLRLIYLALAHMIKFRGHFLIEGDLNPDNSDVDKLFI
QLVQTYNQLFEEKPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLMREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDMIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMMFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQ
LGGD
SEQ ID NO: 28
codon optimized nucleic acid sequences encoding a Cas9 molecule
of S. aureus, and optionally nuclear localization sequences (NLSs)
atgaaaaggaactacattctggggctggacatcgggattacaagcgtggggtatgggattat
tgactatgaaacaagggacgtgatcgacgcaggcgtcagactgttcaaggaggccaacgtgg
aaaacaatgagggacggagaagcaagaggggagccaggcgcctgaaacgacggagaaggcac
agaatccagagggtgaagaaactgctgttcgattacaacctgctgaccgaccattctgagct
gagtggaattaatccttatgaagccagggtgaaaggcctgagtcagaagctgtcagaggaag
agttttccgcagctctgctgcacctggctaagcgccgaggagtgcataacgtcaatgaggtg
gaagaggacaccggcaacgagctgtctacaaaggaacagatctcacgcaatagcaaagctct
ggaagagaagtatgtcgcagagctgcagctggaacggctgaagaaagatggcgaggtgagag
ggtcaattaataggttcaagacaagcgactacgtcaaagaagccaagcagctgctgaaagtg
cagaaggcttaccaccagctggatcagagcttcatcgatacttatatcgacctgctggagac
tcggagaacctactatgagggaccaggagaagggagccccttcggatggaaagacatcaagg
aatggtacgagatgctgatgggacattgcacctattttccagaagagctgagaagcgtcaag
tacgcttataacgcagatctgtacaacgccctgaatgacctgaacaacctggtcatcaccag
ggatgaaaacgagaaactggaatactatgagaagttccagatcatcgaaaacgtgtttaagc
agaagaaaaagcctacactgaaacagattgctaaggagatcctggtcaacgaagaggacatc
aagggctaccgggtgacaagcactggaaaaccagagttcaccaatctgaaagtgtatcacga
tattaaggacatcacagcacggaaagaaatcattgagaacgccgaactgctggatcagattg
ctaagatcctgactatctaccagagctccgaggacatccaggaagagctgactaacctgaac
agcgagctgacccaggaagagatcgaacagattagtaatctgaaggggtacaccggaacaca
caacctgtccctgaaagctatcaatctgattctggatgagctgtggcatacaaacgacaatc
agattgcaatctttaaccggctgaagctggtcccaaaaaaggtggacctgagtcagcagaaa
gagatcccaaccacactggtggacgatttcattctgtcacccgtggtcaagcggagcttcat
ccagagcatcaaagtgatcaacgccatcatcaagaagtacggcctgcccaatgatatcatta
tcgagctggctagggagaagaacagcaaggacgcacagaagatgatcaatgagatgcagaaa
cgaaaccggcagaccaatgaacgcattgaagagattatccgaactaccgggaaagagaacgc
aaagtacctgattgaaaaaatcaagctgcacgatatgcaggagggaaagtgtctgtattctc
tggaggcctccccctggaggacctgctgaacaatccattcaactacgaggtcgatcatatta
tccccagaagcgtgtccttcgacaattcctttaacaacaaggtgctggtcaagcaggaagag
aactctaaaaagggcaataggactcctttccagtacctgtctagttcagattccaagatctc
ttacgaaacctttaaaaagcacattctgaatctggccaaaggaaagggccgcatcagcaaga
ccaaaaaggagtacctgctggaagagcgggacatcaacagattctccgtccagaaggatttt
attaaccggaatctggtggacacaagatacgctactcgcggcctgatgaatctgctgcgatc
ctatttccgggtgaacaatctggatgtgaaagtcaagtccatcaacggcgggttcacatctt
ttctgaggcgcaaatggaagtttaaaaaggagcgcaacaaagggtacaagcaccatgccgaa
gatgctctgattatcgcaaatgccgacttcatctttaaggagtggaaaaagctggacaaagc
caagaaagtgatggagaaccagatgttcgaagagaagcaggccgaatctatgcccgaaatcg
agacagaacaggagtacaaggagattttcatcactcctcaccagatcaagcatatcaaggat
ttcaaggactacaagtactctcaccgggtggataaaaagcccaacagagagctgatcaatga
caccctgtatagtacaagaaaagacgataaggggaataccctgattgtgaacaatctgaacg
gactgtacgacaaagataatgacaagctgaaaaagctgatcaacaaaagtcccgagaagctg
ctgatgtaccaccatgatcctcagacatatcagaaactgaagctgattatggagcagtacgg
cgacgagaagaacccactgtataagtactatgaagagactgggaactacctgaccaagtata
gcaaaaaggataatggccccgtgatcaagaagatcaagtactatgggaacaagctgaatgcc
catctggacatcacagacgattaccctaacagtcgcaacaaggtggtcaagctgtcactgaa
gccatacagattcgatgtctatctggacaacggcgtgtataaatttgtgactgtcaagaatc
tggatgtcatcaaaaaggagaactactatgaagtgaatagcaagtgctacgaagaggctaaa
aagctgaaaaagattagcaaccaggcagagttcatcgcctccttttacaacaacgacctgat
taagatcaatggcgaactgtatagggtcatcggggtgaacaatgatctgctgaaccgcattg
aagtgaatatgattgacatcacttaccgagagtatctggaaaacatgaatgataagcgcccc
cctcgaattatcaaaacaattgcctctaagactcagagtatcaaaaagtactcaaccgacat
tctgggaaacctgtatgaggtgaagagcaaaaagcaccctcagattatcaaaaagggc
SEQ ID NO: 29
codon optimized nucleic acid sequences encoding a Cas9
molecule of S. aureus, and optionally nuclear
localization sequences (NLSs)
atgaagcggaactacatcctgggcctggacatcggcatcaccagcgtgggctacggcatcat
cgactacgagacacgggacgtgatcgatgccggcgtgcggctgttcaaagaggccaacgtgg
aaaacaacgagggcaggcggagcaagagaggcgccagaaggctgaagcggcggaggcggcat
agaatccagagagtgaagaagctgctgttcgactacaacctgctgaccgaccacagcgagct
gagcggcatcaacccctacgaggccagagtgaagggcctgagccagaagctgagcgaggaag
agttctctgccgccctgctgcacctggccaagagaagaggcgtgcacaacgtgaacgaggtg
gaagaggacaccggcaacgagctgtccaccaaagagcagatcagccggaacagcaaggccct
ggaagagaaatacgtggccgaactgcagctggaacggctgaagaaagacggcgaagtgcggg
gcagcatcaacagattcaagaccagcgactacgtgaaagaagccaaacagctgctgaaggtg
cagaaggcctaccaccagctggaccagagcttcatcgacacctacatcgacctgctggaaac
ccggcggacctactatgagggacctggcgagggcagccccttcggctggaaggacatcaaag
aatggtacgagatgctgatgggccactgcacctacttccccgaggaactgcggagcgtgaag
tacgcctacaacgccgacctgtacaacgccctgaacgacctgaacaatctcgtgatcaccag
ggacgagaacgagaagctggaatattacgagaagttccagatcatcgagaacgtgttcaagc
agaagaagaagcccaccctgaagcagatcgccaaagaaatcctcgtgaacgaagaggatatt
aagggctacagagtgaccagcaccggcaagcccgagttcaccaacctgaaggtgtaccacga
catcaaggacattaccgcccggaaagagattattgagaacgccgagctgctggatcagattg
ccaagatcctgaccatctaccagagcagcgaggacatccaggaagaactgaccaatctgaac
tccgagctgacccaggaagagatcgagcagatctctaatctgaagggctataccggcaccca
caacctgagcctgaaggccatcaacctgatcctggacgagctgtggcacaccaacgacaacc
agatcgctatcttcaaccggctgaagctggtgcccaagaaggtggacctgtcccagcagaaa
gagatccccaccaccctggtggacgacttcatcctgagccccgtcgtgaagagaagcttcat
ccagagcatcaaagtgatcaacgccatcatcaagaagtacggcctgcccaacgacatcatta
tcgagctggcccgcgagaagaactccaaggacgcccagaaaatgatcaacgagatgcagaag
cggaaccggcagaccaacgagcggatcgaggaaatcatccggaccaccggcaaagagaacgc
caagtacctgatcgagaagatcaagctgcacgacatgcaggaaggcaagtgcctgtacagcc
tggaagccatccctctggaagatctgctgaacaaccccttcaactatgaggtggaccacatc
atccccagaagcgtgtccttcgacaacagcttcaacaacaaggtgctcgtgaagcaggaaga
aaacagcaagaagggcaaccggaccccattccagtacctgagcagcagcgacagcaagatca
gctacgaaaccttcaagaagcacatcctgaatctggccaagggcaagggcagaatcagcaag
accaagaaagagtatctgctggaagaacgggacatcaacaggttctccgtgcagaaagactt
catcaaccggaacctggtggataccagatacgccaccagaggcctgatgaacctgctgcgga
gctacttcagagtgaacaacctggacgtgaaagtgaagtccatcaatggcggcttcaccagc
tttctgcggcggaagtggaagtttaagaaagagcggaacaaggggtacaagcaccacgccga
ggacgccctgatcattgccaacgccgatttcatcttcaaagagtggaagaaactggacaagg
ccaaaaaagtgatggaaaaccagatgttcgaggaaaagcaggccgagagcatgcccgagatc
gaaaccgagcaggagtacaaagagatcttcatcaccccccaccagatcaagcacattaagga
cttcaaggactacaagtacagccaccgggtggacaagaagcctaatagagagctgattaacg
acaccctgtactccacccggaaggacgacaagggcaacaccctgatcgtgaacaatctgaac
ggcctgtacgacaaggacaatgacaagctgaaaaagctgatcaacaagagccccgaaaagct
gctgatgtaccaccacgacccccagacctaccagaaactgaagctgattatggaacagtacg
gcgacgagaagaatcccctgtacaagtactacgaggaaaccgggaactacctgaccaagtac
tccaaaaaggacaacggccccgtgatcaagaagattaagtattacggcaacaaactgaacgc
ccatctggacatcaccgacgactaccccaacagcagaaacaaggtcgtgaagctgtccctga
agccctacagattcgacgtgtacctggacaatggcgtgtacaagttcgtgaccgtgaagaat
ctggatgtgatcaaaaaagaaaactactacgaagtgaatagcaagtgctatgaggaagctaa
gaagctgaagaagatcagcaaccaggccgagtttatcgcctccttctacaacaacgatctga
tcaagatcaacggcgagctgtatagagtgatcggcgtgaacaacgacctgctgaaccggatc
gaagtgaacatgatcgacatcacctaccgcgagtacctggaaaacatgaacgacaagaggcc
ccccaggatcattaagacaatcgcctccaagacccagagcattaagaagtacagcacagaca
ttctgggcaacctgtatgaagtgaaatctaagaagcaccctcagatcatcaaaaagggc
SEQ ID NO: 30
codon optimized nucleic acid sequences encoding a Cas9 molecule
of S. aureus, and optionally nuclear localization sequences (NLSs)
atgaagcgcaactacatcctcggactggacatcggcattacctccgtgggatacggcatcat
cgattacgaaactagggatgtgatcgacgctggagtcaggctgttcaaagaggcgaacgtgg
agaacaacgaggggcggcgctcaaagaggggggcccgccggctgaagcgccgccgcagacat
agaatccagcgcgtgaagaagctgctgttcgactacaaccttctgaccgaccactccgaact
ttccggcatcaacccatatgaggctagagtgaagggattgtcccaaaagctgtccgaggaag
agttctccgccgcgttgctccacctcgccaagcgcaggggagtgcacaatgtgaacgaagtg
gaagaagataccggaaacgagctgtccaccaaggagcagatcagccggaactccaaggccct
ggaagagaaatacgtggcggaactgcaactggagcggctgaagaaagacggagaagtgcgcg
gctcgatcaaccgcttcaagacctcggactacgtgaaggaggccaagcagctcctgaaagtg
caaaaggcctatcaccaacttgaccagtcctttatcgatacctacatcgatctgctcgagac
tcggcggacttactacgagggtccaggggagggctccccatttggttggaaggatattaagg
agtggtacgaaatgctgatgggacactgcacatacttccctgaggagctgcggagcgtgaaa
tacgcatacaacgcagacctgtacaacgcgctgaacgacctgaacaatctcgtgatcacccg
ggacgagaacgaaaagctcgagtattacgaaaagttccagattattgagaacgtgttcaaac
agaagaagaagccgacactgaagcagattgccaaggaaatcctcgtgaacgaagaggacatc
aagggctatcgagtgacctcaacgggaaagccggagttcaccaatctgaaggtctaccacga
catcaaagacatTaccgcccggaaggagatcattgagaacgcggagctgttggaccagattg
cgaagattctgaccatctaccaatcctccgaggatattcaggaagaactcaccaacctcaac
agcgaactgacccaggaggagatagagcaaatctccaacctgaagggctacaccggaactca
taacctgagcctgaaggccatcaacttgatcctggacgagctgtggcacaccaacgataacc
agatcgctattttcaatcggctgaagctggtccccaagaaagtggacctctcacaacaaaag
gagatccctactacccttgtggacgatttcattctgtcccccgtggtcaagagaagcttcat
acagtcaatcaaagtgatcaatgccattatcaagaaatacggtctgcccaacgacattatca
ttgagctcgcccgcgagaagaactcgaaggacgcccagaagatgattaacgaaatgcagaag
aggaaccgacagactaacgaacggatcgaagaaatcatccggaccaccgggaaggaaaacgc
gaagtacctgatcgaaaagatcaagctccatgacatgcaggaaggaaagtgtctgtactcgc
tggaggccattccgctggaggacttgctgaacaacccttttaactacgaagtggatcatatc
attccgaggagcgtgtcattcgacaattccttcaacaacaaggtcctcgtgaagcaggagga
aaactcgaagaagggaaaccgcacgccgttccagtacctgagcagcagcgactccaagattt
cctacgaaaccttcaagaagcacatcctcaacctggcaaaggggaagggtcgcatctccaag
accaagaaggaatatctgctggaagaaagagacatcaacagattctccgtgcaaaaggactt
catcaaccgcaacctcgtggatactagatacgctactcggggtctgatgaacctcctgagaa
gctactttagagtgaacaatctggacgtgaaggtcaagtcgattaacggaggtttcacctcc
ttcctgcggcgcaagtggaagttcaagaaggaacggaacaagggctacaagcaccacgccga
ggacgccctgatcattgccaacgccgacttcatcttcaaagaatggaagaaacttgacaagg
ctaagaaggtcatggaaaaccagatgttcgaagaaaagcaggccgagtctatgcctgaaatc
gagactgaacaggagtacaaggaaatctttattacgccacaccagatcaaacacatcaagga
tttcaaggattacaagtactcacatcgcgtggacaaaaagccgaacagggaactgatcaacg
acaccctctactccacccggaaggatgacaaagggaataccctcatcgtcaacaaccttaac
ggcctgtacgacaaggacaacgataagctgaagaagctcattaacaagtcgcccgaaaagtt
gctgatgtaccaccacgaccctcagacttaccagaagctcaagctgatcatggagcagtatg
gggacgagaaaaacccgttgtacaagtactacgaagaaactgggaattatctgactaagtac
tccaagaaagataacggccccgtgattaagaagattaagtactacggcaacaagctgaacgc
ccatctggacatcaccgatgactaccctaattcccgcaacaaggtcgtcaagctgagcctca
agccctaccggtttgatgtgtaccttgacaatggagtgtacaagttcgtgactgtgaagaac
cttgacgtgatcaagaaggagaactactacgaagtcaactccaagtgctacgaggaagcaaa
gaagttgaagaagatctcgaaccaggccgagttcattgcctccttctataacaacgacctga
ttaagatcaacggcgaactgtaccgcgtcattggcgtgaacaacgatctcctgaaccgcatc
gaagtgaacatgatcgacatcacttaccgggaatacctggagaatatgaacgacaagcgccc
gccccggatcattaagactatcgcctcaaagacccagtcgatcaagaagtacagcaccgaca
tcctgggcaacctgtacgaggtcaaatcgaagaagcacccccagatcatcaagaaggga
SEQ ID NO: 31
codon optimized nucleic acid sequences encoding a Cas9
molecule of S. aureus, and optionally nuclear
localization sequences (NLSs)
atggccccaaagaagaagcggaaggtcggtatccacggagtcccagcagccaagcggaacta
catcctgggcctggacatcggcatcaccagcgtgggctacggcatcatcgactacgagacac
gggacgtgatcgatgccggcgtgcggctgttcaaagaggccaacgtggaaaacaacgagggc
aggcggagcaagagaggcgccagaaggctgaagcggcggaggcggcatagaatccagagagt
gaagaagctgctgttcgactacaacctgctgaccgaccacagcgagctgagcggcatcaacc
cctacgaggccagagtgaagggcctgagccagaagctgagcgaggaagagttctctgccgcc
ctgctgcacctggccaagagaagaggcgtgcacaacgtgaacgaggtggaagaggacaccgg
caacgagctgtccaccagagagcagatcagccggaacagcaaggccctggaagagaaatacg
tggccgaactgcagctggaacggctgaagaaagacggcgaagtgcggggcagcatcaacaga
ttcaagaccagcgactacgtgaaagaagccaaacagctgctgaaggtgcagaaggcctacca
ccagctggaccagagcttcatcgacacctacatcgacctgctggaaacccggcggacctact
atgagggacctggcgagggcagccccttcggctggaaggacatcaaagaatggtacgagatg
ctgatgggccactgcacctacttccccgaggaactgcggagcgtgaagtacgcctacaacgc
cgacctgtacaacgccctgaacgacctgaacaatctcgtgatcaccagggacgagaacgaga
agctggaatattacgagaagttccagatcatcgagaacgtgttcaagcagaagaagaagccc
accctgaagcagatcgccaaagaaatcctcgtgaacgaagaggatattaagggctacagagt
gaccagcaccggcaagcccgagttcaccaacctgaaggtgtaccacgacatcaaggacatta
ccgcccggaaagagattattgagaacgccgagctgctggatcagattgccaagatcctgacc
atctaccagagcagcgaggacatccaggaagaactgaccaatctgaactccgagctgaccca
ggaagagatcgagcagatctctaatctgaagggctataccggcacccacaacctgagcctga
aggccatcaacctgatcctggacgagctgtggcacaccaacgacaaccagatcgctatcttc
aaccggctgaagctggtgcccaagaaggtggacctgtcccagcagaaagagatccccaccac
cctggtggacgacttcatcctgagccccgtcgtgaagagaagcttcatccagagcatcaaag
tgatcaacgccatcatcaagaagtacggcctgcccaacgacatcattatcgagctggcccgc
gagaagaactccaaggacgcccagaaaatgatcaacgagatgcagaagcggaaccggcagac
caacgagcggatcgaggaaatcatccggaccaccggcaaagagaacgccaagtacctgatcg
agaagatcaagctgcacgacatgcaggaaggcaagtgcctgtacagcctggaagccatccct
ctggaagatctgctgaacaaccccttcaactatgaggtggaccacatcatccccagaagcgt
gtccttcgacaacagcttcaacaacaaggtgctcgtgaagcaggaagaaaacagcaagaagg
gcaaccggaccccattccagtacctgagcagcagcgacagcaagatcagctacgaaaccttc
aagaagcacatcctgaatctggccaagggcaagggcagaatcagcaagaccaagaaagagta
tctgctggaagaacgggacatcaacaggttctccgtgcagaaagacttcatcaaccggaacc
tggtggataccagatacgccaccagaggcctgatgaacctgctgcggagctacttcagagtg
aacaacctggacgtgaaagtgaagtccatcaatggcggcttcaccagctttctgcggcggaa
gtggaagtttaagaaagagcggaacaaggggtacaagcaccacgccgaggacgccctgatca
ttgccaacgccgatttcatcttcaaagagtggaagaaactggacaaggccaaaaaagtgatg
gaaaaccagatgttcgaggaaaggcaggccgagagcatgcccgagatcgaaaccgagcagga
gtacaaagagatcttcatcaccccccaccagatcaagcacattaaggacttcaaggactaca
agtacagccaccgggtggacaagaagcctaatagagagctgattaacgacaccctgtactcc
acccggaaggacgacaagggcaacaccctgatcgtgaacaatctgaacggcctgtacgacaa
ggacaatgacaagctgaaaaagctgatcaacaagagccccgaaaagctgctgatgtaccacc
acgacccccagacctaccagaaactgaagctgattatggaacagtacggcgacgagaagaat
cccctgtacaagtactacgaggaaaccgggaactacctgaccaagtactccaaaaaggacaa
cggccccgtgatcaagaagattaagtattacggcaacaaactgaacgcccatctggacatca
ccgacgactaccccaacagcagaaacaaggtcgtgaagctgtccctgaagccctacagattc
gacgtgtacctggacaatggcgtgtacaagttcgtgaccgtgaagaatctggatgtgatcaa
aaaagaaaactactacgaagtgaatagcaagtgctatgaggaagctaagaagctgaagaaga
tcagcaaccaggccgagtttatcgcctccttctacaacaacgatctgatcaagatcaacggc
gagctgtatagagtgatcggcgtgaacaacgacctgctgaaccggatcgaagtgaacatgat
cgacatcacctaccgcgagtacctggaaaacatgaacgacaagaggccccccaggatcatta
agacaatcgcctccaagacccagagcattaagaagtacagcacagacattctgggcaacctg
tatgaagtgaaatctaagaagcaccctcagatcatcaaaaagggcaaaaggccggcggccac
gaaaaaggccggccaggcaaaaaagaaaaag
SEQ ID NO: 32
codon optimized nucleic acid sequences encoding a Cas9
molecule of S. aureus, and optionally nuclear
localization sequences (NLSs)
accggtgccaccatgtacccatacgatgttccagattacgcttcgccgaagaaaaagcgcaa
ggtcgaagcgtccatgaaaaggaactacattctggggctggacatcgggattacaagcgtgg
ggtatgggattattgactatgaaacaagggacgtgatcgacgcaggcgtcagactgttcaag
gaggccaacgtggaaaacaatgagggacggagaagcaagaggggagccaggcgcctgaaacg
acggagaaggcacagaatccagagggtgaagaaactgctgttcgattacaacctgctgaccg
accattctgagctgagtggaattaatccttatgaagccagggtgaaaggcctgagtcagaag
ctgtcagaggaagagttttccgcagctctgctgcacctggctaagcgccgaggagtgcataa
cgtcaatgaggtggaagaggacaccggcaacgagctgtctacaaaggaacagatctcacgca
atagcaaagctctggaagagaagtatgtcgcagagctgcagctggaacggctgaagaaagat
ggcgaggtgagagggtcaattaataggttcaagacaagcgactacgtcaaagaagccaagca
gctgctgaaagtgcagaaggcttaccaccagctggatcagagcttcatcgatacttatatcg
acctgctggagactcggagaacctactatgagggaccaggagaagggagccccttcggatgg
aaagacatcaaggaatggtacgagatgctgatgggacattgcacctattttccagaagagct
gagaagcgtcaagtacgcttataacgcagatcttacaacgccctgaatgacctgaacaacct
ggtcatcaccagggatgaaaacgagaaactggaatactatgagaagttccagatcatcgaaa
acgtgtttaagcagaagaaaaagcctacactgaaacagattgctaaggagatcctggtcaac
gaagaggacatcaagggctaccgggtgacaagcactggaaaaccagagttcaccaatctgaa
agtgtatcacgatattaaggacatcacagcacggaaagaaatcattgagaacgccgaactgc
tggatcagattgctaagatcctgactatctaccagagctccgaggacatccaggaagagctg
actaacctgaacagcgagctgacccaggaagagatcgaacagattagtaatctgaaggggta
caccggaacacacaacctgtccctgaaagctatcaatctgattctggatgagctgtggcata
caaacgacaatcagattgcaatctttaaccggctgaagctggtcccaaaaaaggtggacctg
agtcagcagaaagagatcccaaccacactggtggacgatttcattctgtcacccgtggtcaa
gcggagcttcatccagagcatcaaagtgatcaacgccatcatcaagaagtacggcctgccca
atgatatcattatcgagctggctagggagaagaacagcaaggacgcacagaagatgatcaat
gagatgcagaaacgaaaccggcagaccaatgaacgcattgaagagattatccgaactaccgg
gaaagagaacgcaaagtacctgattgaaaaaatcaagctgcacgatatgcaggagggaaagt
gtctgtattctctggaggccatccccctggaggacctgctgaacaatccattcaactacgag
gtcgatcatattatccccagaagcgtgtccttcgacaattcctttaacaacaaggtgctggt
caagcaggaagagaactctaaaaagggcaataggactcctttccagtacctgtctagttcag
attccaagatctcttacgaaacctttaaaaagcacattctgaatctggccaaaggaaagggc
cgcatcagcaagaccaaaaaggagtacctgctggaagagcgggacatcaacagattctccgt
ccagaaggattttattaaccggaatctggtggacacaagatacgctactcgcggcctgatga
atctgctgcgatcctatttccgggtgaacaatctggatgtgaaagtcaagtccatcaacggc
gggttcacatcttttctgaggcgcaaatggaagtttaaaaaggagcgcaacaaagggtacaa
gcaccatgccgaagatgctctgattatcgcaaatgccgacttcatctttaaggagtggaaaa
agctggacaaagccaagaaagtgatggagaaccagatgttcgaagagaagcaggccgaatct
atgcccgaaatcgagacagaacaggagtacaaggagattttcatcactcctcaccagatcaa
gcatatcaaggatttcaaggactacaagtactctcaccgggtggataaaaagcccaacagag
agctgatcaatgacaccctgtatagtacaagaaaagacgataaggggaataccctgattgtg
aacaatctgaacggactgtacgacaaagataatgacaagctgaaaaagctgatcaacaaaag
tcccgagaagctgctgatgtaccaccatgatcctcagacatatcagaaactgaagctgatta
tggagcagtacggcgacgagaagaacccactgtataagtactatgaagagactgggaactac
ctgaccaagtatagcaaaaaggataatggccccgtgatcaagaagatcaagtactatgggaa
caagctgaatgcccatctggacatcacagacgattaccctaacagtcgcaacaaggtggtca
agctgtcactgaagccatacagattcgatgtctatctggacaacggcgtgtataaatttgtg
actgtcaagaatctggatgtcatcaaaaaggagaactactatgaagtgaatagcaagtgcta
cgaagaggctaaaaagctgaaaaagattagcaaccaggcagagttcatcgcctccttttaca
acaacgacctgattaagatcaatggcgaactgtatagggtcatcggggtgaacaatgatctg
ctgaaccgcattgaagtgaatatgattgacatcacttaccgagagtatctggaaaacatgaa
tgataagcgcccccctcgaattatcaaaacaattgcctctaagactcagagtatcaaaaagt
actcaaccgacattctgggaaacctgtatgaggtgaagagcaaaaagcaccctcagattatc
aaaaagggctaagaattc
SEQ ID NO: 33
amino acid sequence of an S. aureus Cas9 molecule
MKRNYILGLDIGITSVGYGIIDYETRDVIDAGVRLFKEANVENNEGRRSKRGARRLKRRRRH
RIQRVKKLLFDYNLLTDHSELSGINPYEARVKGLSQKLSEEEFSAALLHLAKRRGVHNVNEV
EEDTGNELSTKEQISRNSKALEEKYVAELQLERLKKDGEVRGSINRFKTSDYVKEAKQLLKV
QKAYHQLDQSFIDTYIDLLETRRTYYEGPGEGSPFGWKDIKEWYEMLMGHCTYFPEELRSVK
YAYNADLYNALNDLNNLVITRDENEKLEYYEKFQIIENVFKQKKKPTLKQIAKEILVNEEDI
KGYRVTSTGKPEFTNLKVYHDIKDITARKEIIENAELLDQIAKILTIYQSSEDIQEELTNLN
SELTQEEIEQISNLKGYTGTHNLSLKAINLILDELWHTNDNQIAIFNRLKLVPKKVDLSQQK
EIPTTLVDDFILSPVVKRSFIQSIKVINAIIKKYGLPNDIIIELAREKNSKDAQKMINEMQK
RNRQTNERIEEIIRTTGKENAKYLIEKIKLHDMQEGKCLYSLEAIPLEDLLNNPFNYEVDHI
IPRSVSFDNSFNNKVLVKQEENSKKGNRTPFQYLSSSDSKISYETFKKHILNLAKGKGRISK
TKKEYLLEERDINRFSVQKDFINRNLVDTRYATRGLMNLLRSYFRVNNLDVKVKSINGGFTS
FLRRKWKFKKERNKGYKHHAEDALIIANADFIFKEWKKLDKAKKVMENQMFEEKQAESMPEI
ETEQEYKEIFITPHQIKHIKDFKDYKYSHRVDKKPNRELINDTLYSTRKDDKGNTLIVNNLN
GLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKPYRFDVYLDNGVYKFVTVKN
LDVIKKENYYEVNSKCYEEAKKLKKISNQAEFIASFYNNDLIKINGELYRVIGVNNDLLNRI
EVNMIDITYREYLENMNDKRPPRIIKTIASKTQSIKKYSTDILGNLYEVKSKKHPQIIKKG
SEQ ID NO: 34
DNA encoding mutant S. aureus Cas9 (D10A)
atgaaaaggaactacattctggggctggccatcgggattacaagcgtggggtatgggattat
tgactatgaaacaagggacgtgatcgacgcaggcgtcagactgttcaaggaggccaacgtgg
aaaacaatgagggacggagaagcaagaggggagccaggcgcctgaaacgacggagaaggcac
agaatccagagggtgaagaaactgctgttcgattacaacctgctgaccgaccattctgagct
gagtggaattaatccttatgaagccagggtgaaaggcctgagtcagaagctgtcagaggaag
agttttccgcagctctgctgcacctggctaagcgccgaggagtgcataacgtcaatgaggtg
gaagaggacaccggcaacgagctgtctacaaaggaacagatctcacgcaatagcaaagctct
ggaagagaagtatgtcgcagagctgcagctggaacggctgaagaaagatggcgaggtgagag
ggtcaattaataggttcaagacaagcgactacgtcaaagaagccaagcagctgctgaaagtg
cagaaggcttaccaccagctggatcagagcttcatcgatacttatatcgacctgctggagac
tcggagaacctactatgagggaccaggagaagggagccccttcggatggaaagacatcaagg
aatggtacgagatgctgatgggacattgcacctattttccagaagagctgagaagcgtcaag
tacgcttataacgcagatctgtacaacgccctgaatgacctgaacaacctggtcatcaccag
ggatgaaaacgagaaactggaatactatgagaagttccagatcatcgaaaacgtgtttaagc
agaagaaaaagcctacactgaaacagattgctaaggagatcctggtcaacgaagaggacatc
aagggctaccgggtgacaagcactggaaaaccagagttcaccaatctgaaagtgtatcacga
tattaaggacatcacagcacggaaagaaatcattgagaacgccgaactgctggatcagattg
ctaagatcctgactatctaccagagctccgaggacatccaggaagagctgactaacctgaac
agcgagctgacccaggaagagatcgaacagattagtaatctgaaggggtacaccggaacaca
caacctgtccctgaaagctatcaatctgattctggatgagctgtggcatacaaacgacaatc
agattgcaatctttaaccggctgaagctggtcccaaaaaaggtggacctgagtcagcagaaa
gagatcccaaccacactggtggacgatttcattctgtcacccgtggtcaagcggagcttcat
ccagagcatcaaagtgatcaacgccatcatcaagaagtacggcctgcccaatgatatcatta
tcgagctggctagggagaagaacagcaaggacgcacagaagatgatcaatgagatgcagaaa
cgaaaccggcagaccaatgaacgcattgaagagattatccgaactaccgggaaagagaacgc
aaagtacctgattgaaaaaatcaagctgcacgatatgcaggagggaaagtgtctgtattctc
tggaggccatccccctggaggacctgctgaacaatccattcaactacgaggtcgatcatatt
atccccagaagcgtgtccttcgacaattcctttaacaacaaggtgctggtcaagcaggaaga
gaactctaaaaagggcaataggactcctttccagtacctgtctagttcagattccaagatct
cttacgaaacctttaaaaagcacattctgaatctggccaaaggaaagggccgcatcagcaag
accaaaaaggagtacctgctggaagagcgggacatcaacagattctccgtccagaaggattt
tattaaccggaatctggtggacacaagatacgctactcgcggcctgatgaatctgctgcgat
cctatttccgggtgaacaatctggatgtgaaagtcaagtccatcaacggcgggttcacatct
tttctgaggcgcaaatggaagtttaaaaaggagcgcaacaaagggtacaagcaccatgccga
agatgctctgattatcgcaaatgccgacttcatctttaaggagtggaaaaagctggacaaag
ccaagaaagtgatggagaaccagatgttcgaagagaagcaggccgaatctatgcccgaaatc
gagacagaacaggagtacaaggagattttcatcactcctcaccagatcaagcatatcaagga
tttcaaggactacaagtactctcaccgggtggataaaaagcccaacagagagctgatcaatg
acaccctgtatagtacaagaaaagacgataaggggaataccctgattgtgaacaatctgaac
ggactgtacgacaaagataatgacaagctgaaaaagctgatcaacaaaagtcccgagaagct
gctgatgtaccaccatgatcctcagacatatcagaaactgaagctgattatggagcagtacg
gcgacgagaagaacccactgtataagtactatgaagagactgggaactacctgaccaagtat
agcaaaaaggataatggccccgtgatcaagaagatcaagtactatgggaacaagctgaatgc
ccatctggacatcacagacgattaccctaacagtcgcaacaaggtggtcaagctgtcactga
agccatacagattcgatgtctatctggacaacggcgtgtataaatttgtgactgtcaagaat
ctggatgtcatcaaaaaggagaactactatgaagtgaatagcaagtgctacgaagaggctaa
aaagctgaaaaagattagcaaccaggcagagttcatcgcctccttttacaacaacgacctga
ttaagatcaatggcgaactgtatagggtcatcggggtgaacaatgatctgctgaaccgcatt
gaagtgaatatgattgacatcacttaccgagagtatctggaaaacatgaatgataagcgccc
ccctcgaattatcaaaacaattgcctctaagactcagagtatcaaaaagtactcaaccgaca
ttctgggaaacctgtatgaggtgaagagcaaaaagcaccctcagattatcaaaaagggc
SEQ ID NO: 35
DNA encoding mutant S. aureus Cas9 molecule (N580A)
atgaaaaggaactacattctggggctggacatcgggattacaagcgtggggtatgggattat
tgactatgaaacaagggacgtgatcgacgcaggcgtcagactgttcaaggaggccaacgtgg
aaaacaatgagggacggagaagcaagaggggagccaggcgcctgaaacgacggagaaggcac
agaatccagagggtgaagaaactgctgttcgattacaacctgctgaccgaccattctgagct
gagtggaattaatccttatgaagccagggtgaaaggcctgagtcagaagctgtcagaggaag
agttttccgcagctctgctgcacctggctaagcgccgaggagtgcataacgtcaatgaggtg
gaagaggacaccggcaacgagctgtctacaaaggaacagatctcacgcaatagcaaagctct
ggaagagaagtatgtcgcagagctgcagctggaacggctgaagaaagatggcgaggtgagag
ggtcaattaataggttcaagacaagcgactacgtcaaagaagccaagcagctgctgaaagtg
cagaaggcttaccaccagctggatcagagcttcatcgatacttatatcgacctgctggagac
tcggagaacctactatgagggaccaggagaagggagccccttcggatggaaagacatcaagg
aatggtacgagatgctgatgggacattgcacctattttccagaagagctgagaagcgtcaag
tacgcttataacgcagatctgtacaacgccctgaatgacctgaacaacctggtcatcaccag
ggatgaaaacgagaaactggaatactatgagaagttccagatcatcgaaaacgtgtttaagc
agaagaaaaagcctacactgaaacagattgctaaggagatcctggtcaacgaagaggacatc
aagggctaccgggtgacaagcactggaaaaccagagttcaccaatctgaaagtgtatcacga
tattaaggacatcacagcacggaaagaaatcattgagaacgccgaactgctggatcagattg
ctaagatcctgactatctaccagagctccgaggacatccaggaagagctgactaacctgaac
agcgagctgacccaggaagagatcgaacagattagtaatctgaaggggtacaccggaacaca
caacctgtccctgaaagctatcaatctgattctggatgagctgtggcatacaaacgacaatc
agattgcaatctttaaccggctgaagctggtcccaaaaaaggtggacctgagtcagcagaaa
gagatcccaaccacactggtggacgatttcattctgtcacccgtggtcaagcggagcttcat
ccagagcatcaaagtgatcaacgccatcatcaagaagtacggcctgcccaatgatatcatta
tcgagctggctagggagaagaacagcaaggacgcacagaagatgatcaatgagatgcagaaa
cgaaaccggcagaccaatgaacgcattgaagagattatccgaactaccgggaaagagaacgc
aaagtacctgattgaaaaaatcaagctgcacgatatgcaggagggaaagtgtctgtattctc
tggaggccatccccctggaggacctgctgaacaatccattcaactacgaggtcgatcatatt
atccccagaagcgtgtccttcgacaattcctttaacaacaaggtgctggtcaagcaggaaga
ggcctctaaaaagggcaataggactcctttccagtacctgtctagttcagattccaagatct
cttacgaaacctttaaaaagcacattctgaatctggccaaaggaaagggccgcatcagcaag
accaaaaaggagtacctgctggaagagCgggacatcaacagattctccgtccagaaggattt
tattaaccggaatctggtggacacaagatacgctactcgcggcctgatgaatctgctgcgat
cctatttccgggtgaacaatctggatgtgaaagtcaagtccatcaacggcgggttcaCatct
tttctgaggcgcaaatggaagtttaaaaaggagcgcaacaaagggtacaagcaccatgccga
agatgctctgattatcgcaaatgccgacttcatctttaaggagtggaaaaagctggacaaag
ccaagaaagtgatggagaaccagatgttcgaagagaagcaggccgaatctatgcccgaaatC
gagacagaacaggagtacaaggagattttcatcactcctcaccagatcaagcatatcaagga
tttcaaggactacaagtactctcaccgggtggataaaaagcccaacagagagctgatcaatg
acaccctgtatagtacaagaaaagacgataaggggaataccctgattgtgaacaatctgaac
ggactgtacgacaaagataatgacaagctgaaaaagctgatcaacaaaagtcccgagaagct
gctgatgtaccaccatgatcctcagacatatcagaaactgaagctgattatggagcagtacg
gcgacgagaagaacccactgtataagtactatgaagagactgggaactacctgaccaagtat
agcaaaaaggataatggccccgtgatcaagaagatcaagtactatgggaacaagctgaatgc
ccatctggacatcacagacgattaccctaacagtcgcaacaaggtggtcaagctgtcactga
agccatacagattcgatgtctatctggacaacggcgtgtataaatttgtgactgtcaagaat
ctggatgtcatcaaaaaggagaactactatgaagtgaatagcaagtgctacgaagaggctaa
aaaGCtgaaaaagattagcaaccaggcagagttcatcgcctccttttacaacaacgacctga
ttaagatcaatggcgaactgtatagggtcatcggggtgaacaatgatctgctgaaccgcatt
gaagtgaatatgattgacatcacttaccgagagtatctggaaaacatgaatgataagcgccc
ccctcgaattatcaaaacaattgcctctaagactcagagtatcaaaaagtactcaaccgaca
ttctgggaaacctgtatgaggtgaagagcaaaaagcaccctcagattatcaaaaagggc
SEQ ID NO: 36
NGGNG
SEQ ID NO: 37
pDO240-179 (plasmid with JCR179)
ctaaattgtaagcgttaatattttgttaaaattcgcgttaaatttttgttaaatcagctcat
tttttaaccaataggccgaaatcggcaaaatcccttataaatcaaaagaatagaccgagata
gggttgagtgttgttccagtttggaacaagagtccactattaaagaacgtggactccaacgt
caaagggcgaaaaaccgtctatcagggcgatggcccactacgtgaaccatcaccctaatcaa
gttttttggggtcgaggtgccgtaaagcactaaatcggaaccctaaagggagcccccgattt
agagcttgacggggaaagccggcgaacgtggcgagaaaggaagggaagaaagcgaaaggagc
gggcgctagggcgctggcaagtgtagcggtcacgctgcgcgtaaccaccacacccgccgcgc
ttaatgcgccgctacagggcgcgtcccattcgccattcaggctgcgcaactgttgggaaggg
cgatcggtgcgggcctcttcgctattacgccagctggcgaaagggggatgtgctgcaaggcg
attaagttgggtaacgccagggttttcccagtcacgacgttgtaaaacgacggccagtgagc
gcgcgtaatacgactcactatagggcgaattgggtacCAAGCTTgcctatttcccatgattc
cttcatatttgcatatacgatacaaggctgttagagagataattggaattaatttgactgta
aacacaaagatattagtacaaaatacgtgacgtagaaagtaataatttcttgggtagtttgc
agttttaaaattatgttttaaaatggactatcatatgcttaccgtaacttgaaagtatttcg
atttcttggctttatatatcttgTGGAASGGACGAAACACCaacacacagCTGGGTtatcag
aggttttagtactctggaaacagaatctactaaaacaaggcaaaatgccgtgtttatctcgt
caacttgttggcgagatttttttGCGGCCGCCCgcggtggagctccagcttttgttcccttt
agtgagggttaattgcgcgcttggcgtaatcatggtcatagctgtttcctgtgtgaaattgt
tatccgctcacaattccacacaacatacgagccggaagcataaagtgtaaagcctggggtgc
ctaatgagtgagctaactcacattaattgcgttgcgctcactgcccgctttccagtcgggaa
acctgtcgtgccagctgcattaatgaatcggccaacgcgcggggagaggcggtttgcgtatt
gggcgctcttccgcttcctcgctcactgactcgctgcgctcggtcgttcggctgcggcgagc
ggtatcagctcactcaaaggcggtaatacggttatccacagaatcaggggataacgcaggaa
agaacatgtgagcaaaaggccagcaaaaggccaggaaccgtaaaaaggccgcgttgctggcg
tttttccataggctccgcccccctgacgagcatcacaaaaatcgacgctcaagtcagaggtg
gcgaaacccgacaggactataaagataccaggcgtttccccctggaagctccctcgtgcgct
ctcctgttccgaccctgccgcttaccggatacctgtccgcctttctcccttcgggaagcgtg
gcgctttctcatagctcacgctgtaggtatctcagttcggtgtaggtcgttcgctccaagct
gggctgtgtgcacgaaccccccgttcagcccgaccgctgcgccttatccggtaactatcgtc
ttgagtccaacccggtaagacacgacttatcgccactggcagcagccactggtaacaggatt
agcagagcgaggtatgtaggcggtgctacagagttcttgaagtggtggcctaactacggcta
cactagaaggacagtatttggtatctgcgctctgctgaagccagttaccttcggaaaaagag
ttggtagctcttgatccggcaaacaaaccaccgctggtagcggtggtttttttgtttgcaag
cagcagattacgcgcagaaaaaaaggatctcaagaagatcctttgatcttttctacggggtc
tgacgctcagtggaacgaaaactcacgttaagggattttggtcatgagattatcaaaaagga
tcttcacctagatccttttaaattaaaaatgaagttttaaatcaatctaaagtatatatgag
taaacttggtctgacagttaccaatgcttaatcagtgaggcacctatctcagcgatctgtct
atttcgttcatccatagttgcctgactccccgtcgtgtagataactacgatacgggagggct
taccatctggccccagtgctgcaatgataccgcgagacccacgctcaccggctccagattta
tcagcaataaaccagccagccggaagggccgagcgcagaagtggtcctgcaactttatccgc
ctccatccagtctattaattgttgccgggaagctagagtaagtagttcgccagttaatagtt
tgcgcaacgttgttgccattgctacaggcatcgtggtgtcacgctcgtcgtttggtatggct
tcattcagctccggttcccaacgatcaaggcgagttacatgatcccccatgttgtgcaaaaa
agcggttagctccttcggtcctccgatcgttgtcagaagtaagttggccgcagtgttatcac
tcatggttatggcagcactgcataattctcttactgtcatgccatccgtaagatgcttttct
gtgactggtgagtactcasccaagtcattctgagaatagtgtatgcggcgaccgagttgctc
ttgcccggcgtcaatacgggataataccgcgccacatagcagaactttaaaagtgctcatca
ttggaaaacgttcttcggggcgaaaactctcaaggatcttaccgctgttgagatccagttcg
atgtaacccactcgtgcacccaactgatcttcagcatcttttactttcaccagcgtttctgg
gtgagcaaaaacaggaaggcaaaatgccgcaaaaaagggaataagggcgacacggaaatgtt
gaatactcatactcttcctttttcaatattattgaagcatttatcagggttattgtctcatg
agcggatacatatttgaatgtatttagaaaaataaacaaataggggttccgcgcacatttcc
ccgaaaagtgccac
SEQ ID NO: 38
pDO240-183 (plasmid with JCR183)
ctaaattgtaagcgttaatattttgttaaaattcgcgttaaatttttgttaaatcagctcat
tttttaaccaataggccgaaatcggcaaaatcccttataaatcaaaagaatagaccgagata
gggttgagtgttgttccagtttggaacaagagtccactattaaagaacgtggactccaacgt
caaagggcgaaaaaccgtctatcagggcgatggcccactacgtgaaccatcaccctaatcaa
gttttttggggtcgaggtgccgtaaagcactaaatcggaaccctaaagggagcccccgattt
agagcttgacggggaaagccggcgaacgtggcgagaaaggaagggaagaaagcgaaaggagc
gggcgctagggcgctggcaagtgtagcggtcacgctgcgcgtaaccaccacacccgccgcgc
ttaatgcgccgctacagggcgcgtcccattcgccattcaggctgcgcaactgttgggaaggg
cgatcggtgcgggcctcttcgctattacgccagctggcgaaagggggatgtgctgcaaggcg
attaagttgggtaacgccagggttttcccagtcacgacgttgtaaaacgacggccagtgagc
gcgcgtaatacgactcactatagggcgaattgggtacCAAGCTTgcctatttcccatgattc
cttcatatttgcatatacgatacaaggctgttagagagataattggaattaatttgactgta
aacacaaagatattagtacaaaatacgtgacgtagaaagtaataatttcttgggtagtttgc
agttttaaaattatgttttaaaatggactatcatatgcttaccgtaacttgaaagtatttcg
atttcttggctttatatatcttgTGGAAAGGACGAAACACCagaactggtgggaaatggtct
aggttttagtactctggaaacagaatctactaaaacaaggcaaaatgccgtgtttatctcgt
caacttgttggcgagatttttttGCGGCCGCCCgcggtggagctccagcttttgttcccttt
agtgagggttaattgcgcgcttggcgtaatcatggtcatagctgtttcctgtgtgaaattgt
tatccgctcacaattccacacaacatacgagccggaagcataaagtgtaaagcctggggtgc
ctaatgagtgagctaactcacattaattgcgttgcgctcactgcccgctttccagtcgggaa
acctgtcgtgccagctgcattaatgastcggccaacgcgcggggagaggcggtttgcgtatt
gggcgctcttccgcttcctcgctcactgactcgctgcgctcggtcgttcggctgcggcgagc
ggtatcagctcactcaaaggcggtaatacggttatccacagaatcaggggataacgcaggaa
agaacatgtgagcaaaaggccagcaaaaggccaggaaccgtaaaaaggccgcgttgctggcg
tttttccataggctccgcccccctgacgagcatcacaaaaatcgacgctcaagtcagaggtg
gcgaaacccgacaggactataaagataccaggcgtttccccctggaagctccctcgtgcgct
ctcctgttccgaccctgccgcttaccggatacctgtccgcctttctcccttcgggaagcgtg
gcgctttctcatagctcacgctgtaggtatctcagttcggtgtaggtcgttcgctccaagct
gggctgtgtgcacgaaccccccgttcagcccgaccgctgcgccttatccggtaactatcgtc
ttgagtccaacccggtaagacacgacttatcgccactggcagcagccactggtaacaggatt
agcagagcgaggtatgtaggcggtgctacagagttcttgaagtggtggcctaactacggcta
cactagaaggacagtatttggtatctgcgctctgctgaagccagttaccttcggaaaaagag
ttggtagctcttgatccggcaaacaaaccaccgctggtagcggtggtttttttgtttgcaag
cagcagattacgcgcagaaaaaaaggatctcaagaagatcctttgatcttttctacggggtc
tgacgctcagtggaacgaaaactcacgttaagggattttggtcatgagattatcaaaaagga
tcttcacctagatccttttaaattaaaaatgaagttttaaatcaatctaaagtatatatgag
taaacttggtctgacagttaccaatgcttaatcagtgaggcacctatctcagcgatctgtct
atttcgttcatccatagttgcctgactccccgtcgtgtagataactacgatacgggagggct
taccatctggccccagtgctgcaatgataccgcgagacccacgctcaccggctccagattta
tcagcaataaaccagccagccggaagggccgagcgcagaagtggtcctgcaactttatccgc
ctccatccagtctattaattgttgccgggaagctagagtaagtagttcgccagttaatagtt
tgcgcaacgttgttgccattgctacaggcatcgtggtgtcacgctcgtcgtttggtatggct
tcattcagctccggttcccaacgatcaaggcgagttacatgatcccccatgttgtgcaaaaa
agcggttagctccttcggtcctccgatcgttgtcagaagtaagttggccgcagtgttatcac
tcatggttatggcagcactgcataattctcttactgtcatgccatccgtaagatgcttttct
gtgactggtgagtactcaaccaagtcattctgagaatagtgtatgcggcgaccgagttgctc
ttgcccggcgtcaatacgggataataccgcgccacatagcagaactttaaaagtgctcatca
ttggaaaacgttcttcggggcgaaaactctcaaggatcttaccgctgttgagatccagttcg
atgtaacccactcgtgcacccaactgatcttcagcatcttttactttcaccagcgtttctgg
gtgagcaaaaacaggaaggcaaaatgccgcaaaaaagggaataagggcgacacggaaatgtt
gaatactcatactcttcctttttcaatattattgaagcatttatcagggttattgtctcatg
agcggatacatatttgaatgtatttagaaaaataaacaaataggggttccgcgcacatttcc
ccgaaaagtgccac
SEQ ID NO: 39
PT366 AAV179 (AAV with SaCas9 and gRNA179)
cctgcaggcagctgcgcgctcgctcgctcactgaggccgcccgggcgtcgggcgacctttgg
tcgcccggcctcagtgagcgagcgagcgcgcagagagggagtggccaactccatcactaggg
gttcctgcggcctctagactcgaggcgttgacattgattattgactagttattaatagtaat
caattacggggtcattagttcatagcccatatatggagttccgcgttacataacttacggta
aatggcccgcctggctgaccgcccaacgacccccgcccattgacgtcaataatgacgtatgt
tcccatagtaacgccaatagggactttccattgacgtcaatgggtggagtatttacggtaaa
ctgcccacttggcagtacatcaagtgtatcatatgccaagtacgccccctattgacgtcaat
gacggtaaatggcccgcctggcattatgcccagtacatgaccttatgggactttcctacttg
gcagtacatctacgtattagtcatcgctattaccatggtgatgcggttttggcagtacatca
atgggcgtggatagcggtttgactcacggggatttccaagtctccaccccattgacgtcaat
gggagtttgttttggcaccaaaatcaacgggactttccaaaatgtcgtaacaactccgcccc
attgacgcaaatgggcggtaggcgtgtacggtgggaggtctatataagcagagctctctggc
taactaccggtgccaccATGGCCCCAAAGAAGAAGCGGAAGGTCGGTATCCACGGAGTCCCA
GCAGCCAAGCGGAACTACATCCTGGGCCTGGACATCGGCATCACCAGCGTGGGCTACGGCAT
CATCGACTACGAGACACGGGACGTGATCGATGCCGGCGTGCGGCTGTTCAAAGAGGCCAACG
TGGAAAACAACGAGGGCAGGCGGAGCAAGAGAGGCGCCAGAAGGCTGAAGCGGCGGAGGCGG
CATAGAATCCAGAGAGTGAAGAAGCTGCTGTTCGACTACAACCTGCTGACCGACCACAGCGA
GCTGAGCGGCATCAACCCCTACGAGGCCAGAGTGAAGGGCCTGAGCCAGAAGCTGAGCGAGG
AAGAGTTCTCTGCCGCCCTGCTGCACCTGGCCAAGAGAAGAGGCGTGCACAACGTGAACGAG
GTGGAAGAGGACACCGGCAACGAGCTGTCCACCAAAGAGCAGATCAGCCGGAACAGCAAGGC
CCTGGAAGAGAAATACGTGGCCGAACTGCAGCTGGAACGGCTGAAGAAAGACGGCGAAGTGC
GGGGCAGCATCAACAGATTCAAGACCAGCGACTACGTGAAAGAAGCCAAACAGCTGCTGAAG
GTGCAGAAGGCCTACCACCAGCTGGACCAGAGCTTCATCGACACCTACATCGACCTGCTGGA
AACCCGGCGGACCTACTATGAGGGACCTGGCGAGGGCAGCCCCTTCGGCTGGAAGGACATCA
AAGAATGGTACGAGATGCTGATGGGCCACTGCACCTACTTCCCCGAGGAACTGCGGAGCGTG
AAGTACGCCTACAACGCCGACCTGTACAACGCCCTGAACGACCTGAACAATCTCGTGATCAC
CAGGGACGAGAACGAGAAGCTGGAATATTACGAGAAGTTCCAGATCATCGAGAACGTGTTCA
AGCAGAAGAAGAAGCCCACCCTGAAGCAGATCGCCAAAGAAATCCTCGTGAACGAAGAGGAT
ATTAAGGGCTACAGAGTGACCAGCACCGGCAAGCCCGAGTTCACCAACCTGAAGGTGTACCA
CGACATCAAGGACATTACCGCCCGGAAAGAGATTATTGAGAACGCCGAGCTGCTGGATCAGA
TTGCCAAGATCCTGACCATCTACCAGAGCAGCGAGGACATCCAGGAAGAACTGACCAATCTG
AACTCCGAGCTGACCCAGGAAGAGATCGAGCAGATCTCTAATCTGAAGGGCTATACCGGCAC
CCACAACCTGAGCCTGAAGGCCATCAACCTGATCCTGGACGAGCTGTGGCACACCAACGACA
ACCAGATCGCTATCTTCAACCGGCTGAAGCTGGTGCCCAAGAAGGTGGACCTGTCCCAGCAG
AAAGAGATCCCCACCACCCTGGTGGACGACTTCATCCTGAGCCCCGTCGTGAAGAGAAGCTT
CATCCAGAGCATCAAAGTGATCAACGCCATCATCAAGAAGTACGGCCTGCCCAACGACATCA
TTATCGAGCTGGCCCGCGAGAAGAACTCCAAGGACGCCCAGAAAATGATCAACGAGATGCAG
AAGCGGAACCGGCAGACCAACGAGCGGATCGAGGAAATCATCCGGACCACCGGCAAAGAGAA
CGCCAAGTACCTGATCGAGAAGATCAAGCTGCACGACATGCAGGAAGGCAAGTGCCTGTACA
GCCTGGAAGCCATCCCTCTGGAAGATCTGCTGAACAACCCCTTCAACTATGAGGTGGACCAC
ATCATCCCCAGAAGCGTGTCCTTCGACAACAGCTTCAACAACAAGGTGCTCGTGAAGCAGGA
AGAAAACAGCAAGAAGGGCAACCGGACCCCATTCCAGTACCTGAGCAGCAGCGACAGCAAGA
TCAGCTACGAAACCTTCAAGAAGCACATCCTGAATCTGGCCAAGGGCAAGGGCAGAATCAGC
AAGACCAAGAAAGAGTATCTGCTGGAAGAACGGGACATCAACAGGTTCTCCGTGCAGAAAGA
CTTCATCAACCGGAACCTGGTGGATACCAGATACGCCACCAGAGGCCTGATGAACCTGCTGG
GGAGCTACTTCAGAGTGAACAACCTGGACGTGAAAGTGAAGTCCATCAATGGCGGCTTCACC
AGCTTTCTGCGGCGGAAGTGGAAGTTTAAGAAAGAGCGGAACAAGGGGTACAAGCACCACGC
CGAGGACGCCCTGATCATTGCCAACGCCGATTTCATCTTCAAAGAGTGGAAGAAACTGGACA
AGGCCAAAAAAGTGATGGAAAACCAGATGTTCGAGGAAAAGCAGGCCGAGAGCATGCCCGAG
ATCGAAACCGAGCAGGAGTACAAAGAGATCTTCATCACCCCCCACCAGATCAAGCACATTAA
GGACTTCAAGGACTACAAGTACAGCCACCGGGTGGACAAGAAGCCTAATAGAGAGCTGATTA
ACGACACCCTGTACTCCACCCGGAAGGACGACAAGGGCAACACCCTGATCGTGAACAATCTG
AACGGCCTGTACGACAAGGACAATGACAAGCTGAAAAAGCTGATCAACAAGAGCCCCGAAAA
GCTGCTGATGTACCACCACGACCCCCAGACCTACCAGAAACTGAAGCTGATTATGGAACAGT
ACGGCGACGAGAAGAATCCCCTGTACAAGTACTACGAGGAAACCGGGAACTACCTGACCAAG
TACTCCAAAAAGGACAACGGCCCCGTGATCAAGAAGATTAAGTATTACGGCAACAAACTGAA
CGCCCATCTGGACATCACCGACGACTACCCCAACAGCAGAAACAAGGTCGTGAAGCTGTCCC
TGAAGCCCTACAGATTCGACGTGTACCTGGACAATGGCGTGTACAAGTTCGTGACCGTGAAG
AATCTGGATGTGATCAAAAAAGAAAACTACTACGAAGTGAATAGCAAGTGCTATGAGGAAGC
TAAGAAGCTGAAGAAGATCAGCAACCAGGCCGAGTTTATCGCCTCCTTCTACAACAACGATC
TGATCAAGATCAACGGCGAGCTGTATAGAGTGATCGGCGTGAACAACGACCTGCTGAACCGG
ATCGAAGTGAACATGATCGACATCACCTACCGCGAGTACCTGGAAAACATGAACGACAAGAG
GCCCCCCAGGATCATTAAGACAATCGCCTCCAAGACCCAGAGCATTAAGAAGTACAGCACAG
ACATTCTGGGCAACCTGTATGAAGTGAAATCTAAGAAGCACCCTCAGATCATCAAAAAGGGC
AAAAGGCCGGCGGCCACGAAAAAGGCCGGCCAGGCAAAAAAGAAAAAGggatcctacccata
cgatgttccagattacgcttacccatacgatgttccagattacgcttacccatacgatgttc
cagattacgcttaagaattcctagagctcgctgatcagcctcgactgtgccttctagttgcc
agccatctgttgtttgcccctcccccgtgccttccttgaccctggaaggtgccactcccact
gtcctttcctaataaaatgaggaaattgcatcgcattgtctgagtaggtgtcattctattct
ggggggtggggtggggcaggacagcaagggggaggattgggaagagaatagcaggcatgctg
gggaggtaccgagggcctatttcccatgattccttcatatttgcatatacgatacaaggctg
ttagagagataattggaattaatttgactgtaaacacaaagatattagtacaaaatacgtga
cgtagaaagtaataatttcttgggtagtttgcagttttaaaattatgttttaaaatggacta
tcatatgcttaccgtaacttgaaagtatttcgatttcttggctttatatatcttGTGGAAAG
GACGAAACACCgaacacacagCTGGGTtatcagaggttttagtactctggaaacagaatcta
ctaaaacaaggcaaaatgccgtgtttatctcgtcaacttgttggcgagatttttgcggccgc
aggaacccctagtgatggagttggccactccctctctgcgcgctcgctcgctcactgaggcc
gggcgaccaaaggtcgcccgacgcccgggctttgcccgggcggcctcagtgagcgagcgagc
gcgcagctgcctgcaggggcgcctgatgcggtattttctccttacgcatctgtgcggtattt
cacaccgcatacgtcaaagcaaccatagtacgcgccctgtagcggcgcattaagcgcggcgg
gtgtggtggttacgcgcagcgtgaccgctacacttgccagcgccctagcgcccgctcctttc
gctttcttcccttcctttctcgccacgttcgccggctttccccgtcaagctctaaatcgggg
gctccctttagggttccgatttagtgctttacggcacctcgaccccaaaaaacttgatttgg
gtgatggttcacgtagtgggccatcgccctgatagacggtttttcgccctttgacgttggag
tccacgttctttaatagtggactcttgttccaaactggaacaacactcaaccctatctcggg
ctattcttttgatttataagggattttgccgatttcggcctattggttaaaaaatgagctga
tttaacaaaaatttaacgcgaattttaacaaaatattaacgtttacaattttatggtgcact
ctcagtacaatctgctctgatgccgcatagttaagccagccccgacacccgccaacacccgc
tgacgcgccctgacgggcttgtctgctcccggcatccgcttacagacaagctgtgaccgtct
ccgggagctgcatgtgtcagaggttttcaccgtcatcaccgaaacgcgcgagacgaaagggc
ctcgtgatacgcctatttttataggttaatgtcatgataataatggtttcttagacgtcagg
tggcacttttcggggaaatgtgcgcggaacccctatttgtttatttttctaaatacattcaa
atatgtatccgctcatgagacaataaccctgataaatgcttcaataatattgaaaaaggaag
agtatgagtattcaacatttccgtgtcgcccttattcccttttttgcggcattttgccttcc
tgtttttgctcacccagaaacgctggtgaaagtaaaagatgctgaagatcagttgggtgcac
gagtgggttacatcgaactggatctcaacagcggtaagatccttgagagttttcgccccgaa
gaacgttttccaatgatgagcacttttaaagttctgctatgtggcgcggtattatcccgtat
tgacgccgggcaagagcaactcggtcgccgcatacactattctcagaatgacttggttgagt
actcaccagtcacagaaaagcatcttacggatggcatgacagtaagagaattatgcagtgct
gccataaccatgagtgataacactgcggccaacttacttctgacaacgatcggaggaccgaa
ggagctaaccgcttttttgcacaacatgggggatcatgtaactcgccttgatcgttgggaac
cggagctgaatgaagccataccaaacgacgagcgtgacaccacgatgcctgtagcaatggca
acaacgttgcgcaaactattaactggcgaactacttactctagcttcccggcaacaattaat
agactggatggaggcggataaagttgcaggaccacttctgcgctcggcccttccggctggct
ggtttattgctgataaatctggagccggtgagcgtggaagccgcggtatcattgcagcactg
gggccagatggtaagccctcccgtatcgtagttatctacacgacggggagtcaggcaactat
ggatgaacgaaatagacagatcgctgagataggtgcctcactgattaagcattggtaactgt
cagaccaagtttactcatatatactttagattgatttaaaacttcatttttaatttaaaagg
atctaggtgaagatcctttttgataatctcatgaccaaaatcccttaacgtgagttttcgtt
ccactgagcgtcagaccccgtagaaaagatcaaaggatcttcttgagatcctttttttctgc
gcgtaatctgctgcttgcaaacaaaaaaaccaccgctaccagcggtggtttgtttgccggat
caagagctaccaactctttttccgaaggtaactggcttcagcagagcgcagataccaaatac
ggtttattgctgataaatctggagccggtgagcgtggaagccgcggtatcattgcagcactg
tgtccttctagtgtagccgtagttaggccaccacttcaagaactctgtagcaccgcctacat
acctcgctctgctaatcctgttaccagtggctgctgccagtggcgataagtcgtgtcttacc
gggttggactcaagacgatagttaccggataaggcgcagcggtcgggctgaacggggggttc
gtgcacacagcccagcttggagcgaacgacctacaccgaactgagatacctacagcgtgagc
tatgagaaagcgccacgcttcccgaagggagaaaggcggacaggtatccggtaagcggcagg
gtcggaacaggagagcgcacgagggagcttccagggggaaacgcctggtatctttatagtcc
tgtcgggtttcgccacctctgacttgagcgtcgatttttgtgatgctcgtcaggggggcgga
gcctatggaaaaacgccagcaacgcggcctttttacggttcctggccttttgctggcctttt
gctcacatgt
SEQ ID NO: 40
PT366 AAV183 (AAV with SaCas9 and gRNA183)
CctgcaggcagctgCgcgctcgctcgctcactgaggccgcccgggcgtcgggcgacctttgg
tcgcccggcctcagtgagcgagcgagcgcgcagagagggagtggccaactccatcactaggg
gttcctgcggcctctagactcgaggcgttgacattgattattgactagttattaatagtaat
caattacggggtcattagttcatagcccatatatggagttccgcgttacataacttacggta
aatggcccgcctggctgaccgcccaacgacccccgcccattgacgtcaataatgacgtatgt
tcccatagtaacgccaatagggactttccattgacgtcaatgggtggagtatttacggtaaa
ctgcccacttggcagtacatcaagtgtatcatatgccaagtacgccccctattgacgtcaat
gacggtaaatggcccgcctggcattatgcccagtacatgaccttatgggactttcctacttg
gcagtacatctacgtattagtcatcgctattaccatggtgatgcggttttggcagtacatca
atgggcgtggatagcggtttgactcacggggatttccaagtctccaccccattgacgtcaat
gggagtttgttttggcaccaaaatcaacgggactttccaaaatgtcgtaacaactccgcccc
attgacgcaaatgggcggtaggcgtgtacggtgggaggtctatataagcagagctctctggc
taactaccggtgccaccATGGCCCCAAAGAAGAAGCGGAAGGTCGGTATCCACGGAGTCCCA
GCAGCCAAGCGGAACTACATCCTGGGCCTGGACATCGGCATCACCAGCGTGGGCTACGGCAT
CATCGACTACGAGACACGGGACGTGATCGATGCCGGCGTGCGGCTGTTCAAAGAGGCCAACG
TGGAAAACAACGAGGGCAGGCGGAGCAAGAGAGGCGCCAGAAGGCTGAAGCGGCGGAGGCGG
CATAGAATCCAGAGAGTGAAGAAGCTGCTGTTCGACTACAACCTGCTGACCGACCACAGCGA
GCTGAGCGGCATCAACCCCTACGAGGCCAGAGTGAAGGGCCTGAGCCAGAAGCTGAGCGAGG
AAGAGTTCTCTGCCGCCCTGCTGCACCTGGCCAAGAGAAGAGGCGTGCACAACGTGAACGAG
GTGGAAGAGGACACCGGCAACGAGCTGTCCACCAAAGAGCAGATCAGCCGGAACAGCAAGGC
CCTGGAAGAGAAATACGTGGCCGAACTGCAGCTGGAACGGCTGAAGAAAGACGGCGAAGTGC
GGGGCAGCATCAACAGATTCAAGACCAGCGACTACGTGAAAGAAGCCAAACAGCTGCTGAAG
GTGCAGAAGGCCTACCACCAGCTGGACCAGAGCTTCATCGACACCTACATCGACCTGCTGGA
AACCCGGCGGACCTACTATGAGGGACCTGGCGAGGGCAGCCCCTTCGGCTGGAAGGACATCA
AAGAATGGTACGAGATGCTGATGGGCCACTGCACCTACTTCCCCGAGGAACTGCGGAGCGTG
AAGTACGCCTACAACGCCGACCTGTACAACGCCCTGAACGACCTGAACAATCTCGTGATCAC
CAGGGACGAGAACGAGAAGCTGGAATATTACGAGAAGTTCCAGATCATCGAGAACGTGTTCA
AGCAGAAGAAGAAGCCCACCCTGAAGCAGATCGCCAAAGAAATCCTCGTGAACGAAGAGGAT
ATTAAGGGCTACAGAGTGACCAGCACCGGCAAGCCCGAGTTCACCAACCTGAAGGTGTACCA
CGACATCAAGGACATTACCGCCCGGAAAGAGATTATTGAGAACGCCGAGCTGCTGGATCAGA
TTGCCAAGATCCTGACCATCTACCAGAGCAGCGAGGACATCCAGGAAGAACTGACCAATCTG
AACTCCGAGCTGACCCAGGAAGAGATCGAGCAGATCTCTAATCTGAAGGGCTATACCGGCAC
CCACAACCTGAGCCTGAAGGCCATCAACCTGATCCTGGACGAGCTGTGGCACACCAACGACA
ACCAGATCGCTATCTTCAACCGGCTGAAGCTGGTGCCCAAGAAGGTGGACCTGTCCCAGCAG
AAAGAGATCCCCACCACCCTGGTGGACGACTTCATCCTGAGCCCCGTCGTGAAGAGAAGCTT
CATCCAGAGCATCAAAGTGATCAACGCCATCATCAAGAAGTACGGCCTGCCCAACGACATCA
TTATCGAGCTGGCCCGCGAGAAGAACTCCAAGGACGCCCAGAAAATGATCAACGAGATGCAG
AAGCGGAACCGGCAGACCAACGAGCGGATCGAGGAAATCATCCGGACCACCGGCAAAGAGAA
CGCCAAGTACCTGATCGAGAAGATCAAGCTGCACGACATGCAGGAAGGCAAGTGCCTGTACA
GCCTGGAAGCCATCCCTCTGGAAGATCTGCTGAACAACCCCTTCAACTATGAGGTGGACCAC
ATCATCCCCAGAAGCGTGTCCTTCGACAACAGCTTCAACAACAAGGTGCTCGTGAAGCAGGA
AGAAAACAGCAAGAAGGGCAACCGGACCCCATTCCAGTACCTGAGCAGCAGCGACAGCAAGA
TCAGCTACGAAACCTTCAAGAAGCACATCCTGAATCTGGCCAAGGGCAAGGGCAGAATCAGC
AAGACCAAGAAAGAGTATCTGCTGGAAGAACGGGACATCAACAGGTTCTCCGTGCAGAAAGA
CTTCATCAACCGGAACCTGGTGGATACCAGATACGCCACCAGAGGCCTGATGAACCTGCTGC
GGAGCTACTTCAGAGTGAACAACCTGGACGTGAAAGTGAAGTCCATCAATGGCGGCTTCACC
AGCTTTCTGCGGCGGAAGTGGAAGTTTAAGAAAGAGCGGAACAAGGGGTACAAGCACCACGC
CGAGGACGCCCTGATCATTGCCAACGCCGATTTCATCTTCAAAGAGTGGAAGAAACTGGACA
AGGCCAAAAAAGTGATGGAAAACCAGATGTTCGAGGAAAAGCAGGCCGAGAGCATGCCCGAG
ATCGAAACCGAGCAGGAGTACAAAGAGATCTTCATCACCCCCCACCAGATCAAGCACATTAA
GGACTTCAAGGACTACAAGTACAGCCACCGGGTGGACAAGAAGCCTAATAGAGAGCTGATTA
ACGACACCCTGTACTCCACCCGGAAGGACGACAAGGGCAACACCCTGATCGTGAACAATCTG
AACGGCCTGTACGACAAGGACAATGACAAGCTGAAAAAGCTGATCAACAAGAGCCCCGAAAA
GCTGCTGATGTACCACCACGACCCCCAGACCTACCAGAAACTGAAGCTGATTATGGAACAGT
ACGGCGACGAGAAGAATCCCCTGTACAAGTACTACGAGGAAACCGGGAACTACCTGACCAAG
TACTCCAAAAAGGACAACGGCCCCGTGATCAAGAAGATTAAGTATTACGGCAACAAACTGAA
CGCCCATCTGGACATCACCGACGACTACCCCAACAGCAGAAACAAGGTCGTGAAGCTGTCCC
TGAAGCCCTACAGATTCGACGTGTACCTGGACAATGGCGTGTACAAGTTCGTGACCGTGAAG
AATCTGGATGTGATCAAAAAAGAAAACTACTACGAAGTGAATAGCAAGTGCTATGAGGAAGC
TAAGAAGCTGAAGAAGATCAGCAACCAGGCCGAGTTTATCGCCTCCTTCTACAACAACGATC
TGATCAAGATCAACGGCGAGCTGTATAGAGTGATCGGCGTGAACAACGACCTGCTGAACCGG
ATCGAAGTGAACATGATCGACATCACCTACCGCGAGTACCTGGAAAACATGAACGACAAGAG
gccccccaggatcattaagacaatcgcctccaagacccagagcattaagaagtacagcacag
ACATTCTGGGCAACCTGTATGAAGTGAAATCTAAGAAGCACCCTCAGATCATCAAAAAGGGC
AAAAGGCCGGCGGCCACGAAAAAGGCCGGCCAGGCAAAAAAGAAAAAGggatcctacccata
cgatgttccagattacgcttacccatacgatgttccagattacgcttacccatacgatgttc
cagattacgcttaagaattcctagagctcgctgatcagcctcgactgtgccttctagttgcc
agccatctgttgtttgcccctcccccgtgccttccttgaccctggaaggtgccactcccact
gtcctttcctaataaaatgaggaaattgcatcgcattgtctgagtaggtgtcattctattct
ggggggtggggtggggcaggacagcaagggggaggattgggaagagaatagcaggcatgctg
gggaggtaccgagggcctatttcccatgattccttcatatttgcatatacgatacaaggctg
ttagagagataattggaattaatttgactgtaaacacaaagatattagtacaaaatacgtga
cgtagaaagtaataatttcttgggtagtttgcagttttaaaattatgttttaaaatggacta
tcatatgcttaccgtaacttgaaagtatttcgatttcttggctttatatatcttGTGGAAAG
GACGAAACACCgagaactggtgggaaatggtctaggttttagtactctggaaacagaatcta
ctaaaacaaggcaaaatgccgtgtttatctcgtcaacttgttggcgagatttttgcggccgc
aggaacccctagtgatggagttggccactccctctctgcgcgctcgctcgctcactgaggcc
gggcgaccaaaggtcgcccgacgcccgggctttgcccgggcggcctcagtgagcgagcgagc
gcgcagctgcctgcaggggcgcctgatgcggtattttctccttacgcatctgtgcggtattt
cacaccgcatacgtcaaagcaaccatagtacgcgccctgtagcggcgcattaagcgcggcgg
gtgtggtggttacgcgcagcgtgaccgctacacttgccagcgccctagcgcccgctcctttc
gctttcttcccttcctttctcgccacgttcgccggctttccccgtcaagctctaaatcgggg
gctccctttagggttccgatttagtgctttacggcacctcgaccccaaaaaacttgatttgg
gtgatggttcacgtagtgggccatcgccctgatagacggtttttcgccctttgacgttggag
tccacgttctttaatagtggactcttgttccaaactggaacaacactcaaccctatctcggg
ctattcttttgatttataagggattttgccgatttcggcctattggttaaaaaatgagctga
tttaacaaaaatttaacgcgaattttaacaaaatattaacgtttacaattttatggtgcact
ctcagtacaatctgctctgatgccgcatagttaagccagccccgacacccgccaacacccgc
tgacgcgccctgacgggcttgtctgctcccggcatccgcttacagacaagctgtgaccgtct
cagggagctgcatgtgtcagaggttttcaccgtcatcaccgaaacgcgcgagacgaaagggc
ctcgtgatacgcctatttttataggttaatgtcatgataataatggtttcttagacgtcagg
tggcacttttcggggaaatgtgcgcggaacccctatttgtttatttttctaaatacattcaa
atatgtatccgctCatgagacaataaccctgataaatgcttcaataatattgaaaaaggaag
agtatgagtattcaacatttccgtgtcgcccttattcccttttttgcggcattttgccttcc
tgtttttgctcacccagaaacgctggtgaaagtaaaagatgctgaagatcagttgggtgcac
gagtgggttacatcgaactggatctcaacagcggtaagatccttgagagttttcgccccgaa
gaacgttttccaatgatgagcacttttaaagttctgctatgtggcgcggtattatcccgtat
tgacgccgggcaagagcaactcggtcgccgcatacactattctcagaatgacttggttgagt
actcaccagtcacagaaaagcatcttacggatggcatgacagtaagagaattatgcagtgct
gccataaccatgagtgataacactgcggccaacttacttctgacaacgatcggaggaccgaa
ggagctaaccgcttttttgcacaacatgggggatcatgtaactcgccttgatcgttgggaac
cggagctgaatgaagccataccaaacgacgagcgtgacaccacgatgcctgtagcaatggca
acaacgttgcgcaaactattaactggcgaactacttactctagcttcccggcaacaattaat
agactggatggaggcggataaagttgcaggaccacttctgcgctcggcccttccggctggct
ggtttattgctgataaatctggagccggtgagcgtggaagccgcggtatcattgcagcactg
gggccagatggtaagccctcccgtatcgtagttatctacacgacggggagtcaggcaactat
ggatgaacgaaatagacagatcgctgagataggtgcctcactgattaagcattggtaactgt
cagaccaagtttactcatatatactttagattgatttaaaacttcatttttaatttaaaagg
atctaggtgaagatcctttttgataatctcatgaccaaaatcccttaacgtgagttttcgtt
ccactgagcgtcagaccccgtagaaaagatcaaaggatcttcttgagatcctttttttctgc
gcgtaatctgctgcttgcaaacaaaaaaaccaccgctaccagcggtggtttgtttgccggat
caagagctaccaactctttttccgaaggtaactggcttcagcagagcgcagataccaaatac
tgtccttctagtgtagccgtagttaggccaccacttcaagaactctgtagcaccgcctacat
acctcgctctgctaatcctgttaccagtggctgctgccagtggcgataagtcgtgtcttacc
gggttggactcaagacgatagttaccggataaggcgcagcggtcgggctgaacggggggttc
gtgcacacagcccagcttggagcgaacgacctacaccgaactgagatacctacagcgtgagc
tatgagaaagcgccacgcttcccgaagggagaaaggcggacaggtatccggtaagcggcagg
gtcggaacaggagagcgcacgagggagcttccagggggaaacgcctggtatctttatagtcc
tgtcgggtttcgccacctctgacttgagcgtcgatttttgtgatgctcgtcaggggggcgga
gcctatggaaaaacgccagcaacgcggcctttttacggttcctggccttttgctggcctttt
gctcacatgt
SEQ ID NO: 41
JCR94
aacaaatatcccttagtatc
SEQ ID NO: 42
JCR99
aatgtatttcttctattcaa
SEQ ID NO: 43
DNA encoding SaCasS in AAV with NLS
atggccccaaagaagaagcggaaggtcggtatccacggagtcccagcagccaagcggaacta
catcctgggcctggacatcggcatcaccagcgtgggctacggcatcatcgactacgagacac
gggacgtgatcgatgccggcgtgcggctgttcaaagaggccaacgtggaaaacaacgagggc
aggcggagcaagagaggcgccagaaggctgaagcggcggaggCggcatagaatccagagagt
gaagaagctgctgttcgactacaacctgctgaccgaccacagcgagctgagcggcatcaacc
cctacgaggccagagtgaagggcctgagccagaagctgagcgaggaagagttctctgccgcc
ctgctgcacctggccaagagaagaggcgtgcacaacgtgaacgaggtggaagaggacaccgg
caacgagctgtccaccaaagagcagatcagccggaacagcaaggccctggaagagaaatacg
tggccgaactgcagctggaacggctgaagaaagacggcgaagtgcggggcagcatcaacaga
ttcaagaccagcgactacgtgaaagaagccaaacagctgctgaaggtgcagaaggcctacca
ccagctggaccagagcttcatcgacacctacatcgacctgctggaaacccggcggacctact
atgagggacctggcgagggcagccccttcggctggaaggacatcaaagaatggtacgagatg
ctgatgggccactgcacctacttccccgaggaactgcggagcgtgaagtacgcctacaacgc
cgacctgtacaacgccctgaacgacctgaacaatctcgtgatcaccagggacgagaacgaga
agctggaatattacgagaagttccagatcatcgagaacgtgttcaagcagaagaagaagccc
accctgaagcagatcgccaaagaaatcctcgtgaacgaagaggatattaagggctacagagt
gaccagcaccggcaagcccgagttcaccaacctgaaggtgtaccacgacatcaaggacatta
ccgcccggaaagagattattgagaacgccgagctgctggatcagattgccaagatcctgacc
atctaccagagcagcgaggacatccaggaagaactgaccaatctgaactccgagctgaccca
ggaagagatcgagcagatctctaatctgaagggctataccggcacccacaacctgagcctga
aggccatcaacctgatcctggacgagctgtggcacaccaacgacaaccagatcgctatcttc
aaccggctgaagctggtgcccaagaaggtggacctgtcccagcagaaagagatccccaccac
cctggtggacgacttcatcctgagccccgtcgtgaagagaagcttcatccagagcatcaaag
tgatcaacgccatcatcaagaagtacggcctgcccaacgacatcattatcgagctggcccgc
gagaagaactccaaggacgcccagaaaatgatcaacgagatgcagaagcggaaccggcagac
caacgagcggatcgaggaaatcatccggaccaccggcaaagagaacgccaagtacctgatcg
agaagatcaagctgcacgacatgcaggaaggcaagtgcctgtacagcctggaagccatccct
ctggaagatctgctgaacaaccccttcaactatgaggtggaccacatcatccccagaagcgt
gtccttcgacaacagcttcaacaacaaggtgctcgtgaagcaggaagaaaacagcaagaagg
gcaaccggaccccattccagtacctgagcagcagcgacagcaagatcagctacgaaaccttc
aagaagcacatcctgaatctggccaagggcaagggcagaatcagcaagaccaagaaagagta
tctgctggaagaacgggacatcaacaggttctccgtgcagaaagacttcatcaaccggaacc
tggtggataccagatacgccaccagaggcctgatgaacctgctgcggagctacttcagagtg
aacaacctggacgtgaaagtgaagtccatcaatggcggcttcaccagctttctgcggcggaa
gtggaagtttaagaaagagcggaacaaggggtacaagcaccacgccgaggacgccctgatca
ttgccaacgccgatttcatcttcaaagagtggaagaaactggacaaggccaaaaaagtgatg
gaaaaccagatgttcgaggaaaagcaggccgagagcatgcccgagatcgaaaccgagcagga
gtacaaagagatcttcatcaccccccaccagatcaagcacattaaggacttcaaggactaca
agtacagccaccgggtggacaagaagcctaatagagagctgattaacgacaccctgtactcc
acccggaaggacgacaagggcaacaccctgatcgtgaacaatctgaacggcctgtacgacaa
ggacaatgacaagctgaaaaagctgatcaacaagagccccgaaaagctgctgatgtaccacc
acgacccccagacctaccagaaactgaagctgattatggaacagtacggcgacgagaagaat
cccctgtacaagtactacgaggaaaccgggaactacctgaccaagtactccaaaaaggacaa
cggccccgtgatcaagaagattaagtattacggcaacaaactgaacgcccatctggacatca
ccgacgactaccccaacagcagaaacaaggtcgtgaagctgtccctgaagccctacagattc
gacgtgtacctggacaatggcgtgtacaagttcgtgaccgtgaagaatctggatgtgatcaa
aaaagaaaactactacgaagtgaatagcaagtgctatgaggaagctaagaagctgaagaaga
tcagcaaccaggccgagtttatcgcctccttctacaacaacgatctgatcaagatcaacggc
gagctgtatagagtgatcggcgtgaacaacgacctgctgaaccggatcgaagtgaacatgat
cgacatcacctaccgcgagtacctggaaaacatgaacgacaagaggccccccaggatcatta
agacaatcgcctccaagacccagagcattaagaagtacagcacagacattctgggcaacctg
tatgaagtgaaatctaagaagcaccctcagatcatcaaaaagggcaaaaggccggcggccac
gaaaaaggccggccaggcaaaaaagaaaaag
SEQ ID NO: 44
DNA encoding SaCas9 in AAV no NLS
aagcggaactacatcctgggcctggacatcggcatcaccagcgtgggctacggcatcatcga
ctacgagacacgggacgtgatcgatgccggcgtgcggctgttcaaagaggccaacgtggaaa
acaacgagggcaggcggagcaagagaggcgccagaaggctgaagcggcggaggcggcataga
atccagagagtgaagaagctgctgttcgactacaacctgctgaccgaccacagcgagctgag
cggcatcaacccctacgaggccagagtgaagggcctgagccagaagctgagcgaggaagagt
tctctgccgccctgctgcacctggccaagagaagaggcgtgcacaacgtgaacgaggtggaa
gaggacaccggcaacgagctgtccaccaaagagcagatcagccggaacagcaaggccctgga
agagaaatacgtggccgaactgcagctggaacggctgaagaaagacggcgaagtgcggggca
gcatcaacagattcaagaccagcgactacgtgaaagaagccaaacagctgctgaaggtgcag
aaggcctaccaccagctggaccagagcttcatcgacacctacatcgacctgctggaaacccg
gcggacctactatgagggacctggcgagggcagccccttcggctggaaggacatcaaagaat
ggtacgagatgctgatgggccactgcacctacttccccgaggaactgcggagcgtgaagtac
gcctacaacgccgacctgtacaacgccctgaacgacctgaacaatctcgtgatcaccaggga
cgagaacgagaagctggaatattacgagaagttccagatcatcgagaacgtgttcaagcaga
agaagaagcccaccctgaagcagatcgccaaagaaatcctcgtgaacgaagaggatattaag
ggctacagagtgaccagcaccggcaagcccgagttcaccaacctgaaggtgtaccacgacat
caaggacattaccgcccggaaagagattattgagaacgccgagctgctggatcagattgcca
agatcctgaccatctaccagagcagcgaggacatccaggaagaactgaccaatctgaactcc
gagctgacccaggaagagatcgagcagatctctaatctgaagggctataccggcacccacaa
cctgagcctgaaggccatcaacctgatcctggacgagctgtggcacaccaacgacaaccaga
tcgctatcttcaaccggctgaagctggtgcccaagaaggtggacctgtcccagcagaaagag
atccccaccaccctggtggacgacttcatcctgagccccgtcgtgaagagaagcttcatcca
gagcatcaaagtgatcaacgccatcatcaagaagtacggcctgcccaacgacatcattatcg
agctggcccgcgagaagaactccaaggacgcccagaaaatgatcaacgagatgcagaagcgg
aaccggcagaccaacgagcggatcgaggaaatcatccggaccaccggcaaagagaacgccaa
gtacctgatcgagaagatcaagctgcacgacatgcaggaaggcaagtgcctgtacagcctgg
aagccatccctctggaagatctgctgaacaaccccttcaactatgaggtggaccacatcatc
cccagaagcgtgtccttcgacaacagcttcaacaacaaggtgctcgtgaagcaggaagaaaa
cagcaagaagggcaaccggaccccattccagtacctgagcagcagcgacagcaagatcagct
acgaaaccttcaagaagcacatcctgaatctggccaagggcaagggcagaatcagcaagacc
aagaaagagtatctgctggaagaacgggacatcaacaggttctccgtgcagaaagacttcat
caaccggaacctggtggataccagatacgccaccagaggcctgatgaacctgctgcggagct
acttcagagtgaacaacctggacgtgaaagtgaagtccatcaatggcggcttcaccagcttt
ctgcggcggaagtggaagtttaagaaagagcggaacaaggggtacaagcaccacgccgagga
cgccctgatcattgccaacgccgatttcatcttcaaagagtggaagaaactggacaaggcca
aaaaagtgatggaaaaccagatgttcgaggaaaagcaggccgagagcatgcccgagatcgaa
accgagcaggagtacaaagagatcttcatcaccccccaccagatcaagcacattaaggactt
caaggactacaagtacagccaccgggtggacaagaagcctaatagagagctgattaacgaca
ccctgtactccacccggaaggacgacaagggcaacaccctgatcgtgaacaatctgaacggc
ctgtacgacaaggacaatgacaagctgaaaaagctgatcaacaagagccccgaaaagctgct
gatgtaccaccacgacccccagacctaccagaaactgaagctgattatggaacagtacggcg
acgagaagaatcccctgtacaagtactacgaggaaaccgggaactacctgaccaagtactcc
aaaaaggacaacggccccgtgatcaagaagattaagtattacggcaacaaactgaacgccca
tctggacatcaccgacgactaccccaacagcagaaacaaggtcgtgaagctgtccctgaagc
cctacagattcgacgtgtacctggacaatggcgtgtacaagttcgtgaccgtgaagaatctg
gatgtgatcaaaaaagaaaactactacgaagtgaatagcaagtgctatgaggaagctaagaa
gctgaagaagatcagcaaccaggccgagtttatcgcctccttctacaacaacgatctgatca
agatcaacggcgagctgtatagagtgatcggcgtgaacaacgacctgctgaaccggatcgaa
gtgaacatgatcgacatcacctaccgcgagtacctggaaaacatgaacgacaagaggccccc
caggatcattaagacaatcgcctccaagacccagagcattaagaagtacagcacagacattc
tgggcaacctgtatgaagtgaaatctaagaagcaccctcagatcatcaaaaagggc
SEQ ID NO: 45
Amino acid sequence of the SaCas9 encoded by SaCas9 in AAV no NLS
KRNYILGLDIGITSVGYGIIDYETRDVIDAGVRLFKEANVENNEGRRSKRGARRLKRRRRHR
IQRVKKLLFDYNLLTDHSELSGINPYEARVKGLSQKLSEEEFSAALLHLAKRRGVHNVNEVE
EDTGNELSTKEQISRNSKALEEKYVAELQLERLKKDGEVRGSINRFKTSDYVKEAKQLLKVQ
KAYHQLDQSFIDTYIDLLETRRTYYEGPGEGSPFGWKDIKEWYEMLMGHCTYFPEELRSVKY
AYNADLYNALNDLNNLVITRDENEKLEYYEKFQIIENVFKQKKKPTLKQIAKEILVNEEDIK
GYRVTSTGKPEFTNLKVYHDIKDITARKEIIENAELLDQIAKILTIYQSSEDIQEELTNLNS
ELTQEEIEQISNLKGYTGTHNLSLKAINLILDELWHTNDNQIAIFNRLKLVPKKVDLSQQKE
IPTTLVDDFILSPVVKRSFIQSIKVINAIIKKYGLPNDIIIELAREKNSKDAQKMINEMQKR
NRQTNERIEEIIRTTGKENAKYLIEKIKLHDMQEGKCLYSLEAIPLEDLLNNPFNYEVDHII
PRSVSFDNSFNNKVLVKQEENSKKGNRTPFQYLSSSDSKISYETFKKHILNLAKGKGRISKT
KKEYLLEERDINRFSVQKDFINRNLVDTRYATRGLMNLLRSYFRVNNLDVKVKSINGGFTSF
LRRKWKFKKERNKGYKHHAEDALIIANADFIFKEWKKLDKAKKVMENQMFEEKQAESMPEIE
TEQEYKEIFITPHQIKHIKDFKDYKYSHRVDKKPNRELINDTLYSTRKDDKGNTLIVNNLNG
LYDKDNDKLKKLINKSPEKLLMYHHDPQTYQKLKLIMEQYGDEKNPLYKYYEETGNYLTKYS
KKDNGPVIKKIKYYGNKLNAHLDITDDYPNSRNKVVKLSLKPYRFDVYLDNGVYKFVTVKNL
DVIKKENYYEVNSKCYEEAKKLKKISNQAEFIASFYNNDLIKINGELYRVIGVNNDLLNRIE
VNMIDITYREYLENMNDKRPPRIIKTIASKTQSIKKYSTDILGNLYEVKSKKHPQIIKKG

| | | |
|---|---|---|
| SEQ ID NO: 46 | Delta 52 | AACGCTGAAGAACCCTGATAATAGAA |
| | | GAAATACATTTTTAAATCAATTCAGG |
| SEQ ID NO: 47 | Founder 76 | AAGAACCCTGATA- |
| | | TTATCTTAGTAGATTA- |
| | | ATAGAAGAAATACATTTTTAAA |
| SEQ ID NO: 48 | Founder 63 | GCTGAAGAACCCTGA------- |
| | | AAAAATACATTTTTTTATCAATTCAG |
| | | A |
| SEQ ID NO: 49 | Founder 7 | GAAT----(19x)-----GAT- |
| | | TTTCTTGTAGAAGAAATAACAATT- |
| | | AAATC |
| SEQ ID NO: 50 | Delta 52 | AACGCTGAAGAACCCTGATAATAGAA |
| | | GAAATACATTTTTAAATCAATTCAGG |
| SEQ ID NO: 51 | 54497 | AACGCTGAAGAACCCTGATATtatct |
| | | tagtagattaATAGAAGAAATACATT |
| | | TTTAAAT |
| SEQ ID NO: 52 | 54498 | AACGCTGAAGAACCCTGATATTATCT |
| | | TAGTAGATTAATAGAAGAAATACATT |
| | | TTTAAAT |
| SEQ ID NO: 53 | Pam Sequence | AAGAGT |
| SEQ ID NO: 54 | Pam Sequence | GGGAAT |
| SEQ ID NO: 55 | Pam Sequence | AGG |
| SEQ ID NO: 56 | Pam Sequence | TGG |
| SEQ ID NO: 57 | Pam Sequence | GAGAGT |
| SEQ ID NO: 58 | Pam Sequence | GGGAGT |
| SEQ ID NO: 59 | Pam Sequence | TTGAAT |
| SEQ ID NO: 60 | Pam Sequence | CAGAGT |
| SEQ ID NO: 61 | Pam Sequence | TGGAGT |
| SEQ ID NO: 62 | Pam Sequence | ATGAGT |
| SEQ ID NO: 63 | Pam Sequence | AGGAAT |
| SEQ ID NO: 64 | Target sequence-JCR89 | AAAGATATATAATGTCATGAAT |
| SEQ ID NO: 65 | Target sequence-JCR91 | GCAGAATCAAATATAATAGTCT |
| SEQ ID NO: 66 | Target sequence-JCR159 | CAATTAAATTTGACTTATTGTT |
| SEQ ID NO: 67 | Target sequence-JCR160 | CTAGACCATTTCCCACCAGTTC |
| SEQ ID NO: 68 | Target sequence-JCR167 | AGGACTTTATTACCAAAGGA |
| SEQ ID NO: 69 | Target sequence-JCR166 | ATCCAAGTCCATTTGATTCCTA |
| SEQ ID NO: 70 | Target sequence-JCR168 | TAATTCTTTCTAGAAAGAGCCT |
| SEQ ID NO: 71 | Target sequence-JCR170 | GGACATGTGCAAGATGCAAGAG |
| SEQ ID NO: 72 | Target sequence-JCR171 | TGTATGTAGAAGACCTCTAAGT |
| SEQ ID NO: 73 | Target sequence-JCR156 | TCCCCTCACCACTCACCTCTGA |
| SEQ ID NO: 74 | Target sequence-JCR157 | CTCTGATAACCCAGUGTGTGI |
| SEQ ID NO: 75 | Target sequence-JCR176 | CTCTGATAACCCAGCTGTG |
| SEQ ID NO: 76 | Target sequence-JCR177 | CTCTGATAACCCAGCTGTGT |
| SEQ ID NO: 77 | Target sequence-JCR178 | CTCTGATAACCCAGCTGTGTG |
| SEQ ID NO: 78 | Target sequence-JCR179 | CTCTGATAACCCAGCTGTGTGTT |
| SEQ ID NO: 79 | Target sequence-JCR180 | CTAGACCATTTCCCACCAG |
| SEQ ID NO: 80 | Target sequence-JCR181 | CTAGACCATTTCCCACCAGT |
| SEQ ID NO: 81 | Target sequence-JCR182 | CTAGACCATTTCCCACCAGTT |
| SEQ ID NO: 82 | Target sequence-JCR183 | CTAGACCATTTCCCACCAGTTCT |

SEQ ID NO: 83
pDO242 (SaCas9 used in all JCR89/91 projects and
JCR157/160 projects for in vitro work)
ctaaattgtaagcgttaatattttgttaaaattogcgttaaatttttgttaaatcagctcat
tttttaaccaataggccgaaatcggcaaaatcccttataaatcaaaagaatagaccgagata
gggttgagtgttgttccagtttggaacaagagtccactattaaagaacgtggactccaacgt
caaagggcgaaaaaccgtctatcagggcgatggcccactacgtgaaccatcaccctaatcaa
gttttttggggtcgaggtgccgtaaagcactaaatcggaaccctaaagggagcccccgattt
agagcttgacggggaaagccggcgaacgtggcgagaaaggaagggaagaaagcgaaaggagc
gggcgctagggcgctggcaagtgtagcggtcacgctgcgcgtaaccaccacacccgccgcgc
ttaatgcgccgctacagggcgcgtcccattcgccattcaggCtgcgcaactgttgggaaggg
cgatcggtgcgggcctcttcgctattacgccagctggcgaaagggggatgtgctgcaaggcg
attaagttgggtaacgccagggttttcccagtcacgacgttgtaaaacgacggccagtgagc
gcgcgtaatacgactcactatagggcgaattgggtacCtttaattctagtactatgcaTgcg
ttgacattgatta-tgactagttattaatagtaatcaattacggggtcattagttcatagcc
catatatggagttcogcgttacataacttacggtaaatggccogcctggctgaccgcccaac
gaccccogcccattgacgtcaataatgacgtatgttcccatagtaacgccaatagggacttt
ccattgacgtcaatgggtggagtatttacggtaaactgcccacttggcagtacatcaagtgt
atcatatgccaagtacgccccctattgacgtcaatgacggtaaatggcccgcctggcattat
gcccagtacatgaccttatgggactttcctacttggcagtacatctacgtattagtcatcgc
tattaccatggtgatgcggttttggcagtacatcaatgggcgtggatagcggtttgactcac
ggggatttccaagtctccaccccattgacgtcaatgggagtttgttttggcaccaaaatcaa
cgggactttccaaaatgtogtaacaactocgccccattgacgcaaatgggcggtaggcgtgt
acggtgggaggtctatataagcagagctctctggctaactaccggtgccaccatgaaaagga
actacattctggggctggacatcgggattacaagcgtggggtatgggattattgactatgaa
acaagggacgtgatcgacgcaggcgtcagactgttcaaggaggccaacgtggaaaacaatga
gggacggagaagcaagaggggagccaggcgcctgaaacgacggagaaggcacagaatccaga
gggtgaagaaactgctgttcgattacaacctgctgaccgaccattctgagctgagtggaatt
aatccttatgaagccagggtgaaaggcctgagtcagaagctgtcagaggaagagttttccgc
agctctgctgcacstggctaagcgccgaggagtgcataacgtcaatgaggtggaagaggaca
ccggcaacgagctgtctacaaaggaacagatctcacgcaatagcaaagctctggaagagaag
tatgtcgcagagctgcagctggaacggctgaagaaagatggcgaggtgagagggtcaattaa
taggttcaagacaagcgactacgtcaaagaagccaagcagctgctgaaagtgcagaaggctt
accaccagctggatcagagcttcatcgatacttatatcgacctgctggagactcggagaaCC
tactatgagggaccaggagaagggagccccttcggatggaaagacatcaaggaatggtaCga
gatgctgatgggacattgcacctattttccagaagagctgagaagcgtcaagtacgcttata
acgcagatctgtacaacgccctgaatgacctgaacaacctggtcatcaccagggatgaaaac
gagaaactggaatactatgagaagttccagatcatcgaaaacgtgtttaagcagaagaaaaa
gcctacactgaaacagattgctaaggagatcctggtcaacgaagaggacatcaagggctacc
gggtgacaagcactggaaaaccagagttcaccaatctgaaagtgtatcacgatattaaggac
atcacagcacggaaagaaatcattgagaacgccgaactgctggatcagattgctaagatcct
gactatctaccagagctccgaggacatccaggaagagctgactaacctgaacagcgagctga
cccaggaagagatcgaacagattagtaatctgaaggggtacaccggaacacacaacctgtcc
ctgaaagctatcaatctgattctggatgagctgtggcatacaaacgacaatcagattgcaat
ctttaaccggctgaagctggtcccaaaaaaggtggacctgagtcagcagaaagagatcccaa
ccacactggtggacgatttcattctgtcacccgtggtcaagcggagcttcatccagagcatc
aaagtgatcaacgccatcatcaagaagtacggcctgcccaatgatatcattatcgagctggc
tagggagaagaacagcaaggacgcacagaagatgatcaatgagatgcagaaacgaaaccggc
agaccaatgaacgcattgaagagattatccgaactaccgggaaagagaacgcaaagtacctg
attgaaaaaatcaagctgcacgatatgcaggagggaaagtgtctgtattctctggaggccat
ccccctggaggacctgctgaacaatccattcaactacgaggtcgatcatattatccccagaa
gcgtgtccttcgacaattcctttaacaacaaggtgctggtcaagcaggaagagaactctaaa
aagggcaataggactcctttccagtacctgtctagttcagattccaagatctcttacgaaac
ctttaaaaagcacattctgaatctggccaaaggaaagggccgcatcagcaagaccaaaaagg
agtacctactggaagagcgggacatcaacagattctccgtccagaaggattttattaaccgg
aatctggtggacacaagatacgctactcgcggcctgatgaatctgctgcgatcctatttccg
ggtgaacaatctggatgtgaaagtcaagtccatcaacggcgggttcacatcttttctgaggc
gcaaatggaagtttaaaaaggagcgcaacaaagggtacaagcaccatgccgaagatgctctg
attatcgcaaatgccgacttcatctttaaggagtggaaaaagctggacaaagccaagaaagt
gatggagaaccagatgttcgaagagaagcaggccgaatctatgcccgaaatcgagacagaac
aggagtacaaggagattttcatcactcctcaccagatcaagcatatcaaggatttcaaggac
tacaagtactctcaccgggtggataaaaagcccaacagagagctgatcaatgacaccctgta
tagtacaagaaaagacgataaggggaataccctgattgtgaacaatctgaacggactgtacg
acaaagataatgacaagctgaaaaagctgatcaacaaaagtcccgagaagctgctgatgtac
caccatgatcctcagacatatcagaaactgaagctgattatggagcagtacggcgacgagaa
gaacccactgtataagtactatgaagagactgggaactacctgaccaagtatagcaaaaagg
ataatggccccgtgatcaagaagatcaagtactatgggaacaagctgaatgcccatctggac
atcacagacgattaccctaacagtcgcaacaaggtggtcaagctgtcactgaagccatacag
attcgatgtctatctggacaacggcgtgtataaatttgtgactgtcaagaatctggatgtca
tcaaaaaggagaactactatgaagtgaatagcaagtgctacgaagaggctaaaaagctgaaa
aagattagcaaccaggcagagttcatcgcctcottttacaacaacgacctgattaagatcaa
tggcgaactgtatagggtcatcggggtgaacaatgatctgctgaaccgcattgaagtgaata
tgattgacatcacttaccgagagtatctggaaaacatgaatgataagcgcccccctcgaatt
atcaaaacaattgcctctaagactcagagtatcaaaaagtactcaaccgacattctgggaaa
cctgtatgaggtgaagagcaaaaagcaccctcagattatcaaaaagggcagcggaggcaagc
gtcctgctgctactaagaaagctggtcaagctaagaaaaagaaaggatcctacccatacgat
gttccagattacgcttaagaattcctagagctcgctgatcagcctcgactgtgccttctagt
tgccagccatctgttgtttgcccctcccccgtgccttccttgaccctggaaggtgccactcc
cactgtcctttcctaataaaatgaggaaattgcatcgcattgtctgagtaggtgtcattcta
ttctggggggtggggtggggcaggacagcaagggggaggattgggaagagaatagcaggcat
gctggggaggtagcggccgcCCgcggtggagctccagcttttgttccctttagtgagggtta
attgcgcgcttggcgtaatcatggtcatagctgtttcctgtgtgaaattgttatccgctcac
aattccacacaacatacgagccggaagcataaagtgtaaagcctggggtgcctaatgagtga
gctaactcacattaattgcgttgcgctcactgcccgctttccagtcgggaaacctgtcgtgc
cagctgcattaatgaatcggccaacgcgcggggagaggcggtttgcgtattgggcgctcttc
cgcttcCtcgctcactgactCgctgcgCtcggtcgttcggctgcggcgagCggtatcagctc
actcaaaggcggtaatacggttatccacagaatcaggggataacgcaggaaagaacatgtga
gcaaaaggccagcaaaaggccaggaaccgtaaaaaggccgcgttgctggcgtttttccatag
gctccgcccccctgacgagcatcacaaaaatcgacgctcaagtcagaggtggcgaaacccga
caggactataaagataccaggcgtttccccctggaagctccctcgtgcgctctcctgttccg
accctgccgcttaccggatacctgtccgcctttctcccttcgggaagcgtggcgctttctca
tagctcacgctgtaggtatctcagttcggtgtaggtcgttcgctccaagctgggctgtgtgc
acgaaccccccgttcagcccgaccgctgcgccttatccggtaactatcgtcttgagtccaac
ccggtaagacacgacttatcgccactggcagcagccactggtaacaggattagcagagcgag
gtatgtaggcggtgctacagagttcttgaagtggtggcctaactacggctacactagaagga
cagtatttggtatctgcgctctgctgaagccagttaccttcggaaaaagagttggtagctct
tgatccggcaaacaaaccaccgctggtagcggtggtttttttgtttgcaagcagcagattac
gcgcagaaaaaaaggatcucaagaagatccttugatcttutctacggggtctgacgctcagt
ggaacgaaaactcacgttaagggattttggtcatgagattatcaaaaaggatcttcacctag
atccttttaaattaaaaatgaagttttaaatcaatctaaagtatatatgagtaaacttggtc
tgacagttaccaatgcttaatcagtgaggcacctatctcagcgatctgtctatttcgttcat
ccatagttgcctgactccccgtcgtgtagataactacgatacgggagggcttaccatctggc
cccagtgctgcaatgataccgcgagacccacgctcaccggctccagatttatcagcaataaa
ccagccagccggaagggccgagcgcagaagtggtcctgcaactttatccgcctccatccagt
ctattaattgttgccgggaagctagagtaagtagttcgccagttaatagtttgcgcaacgtt
gttgccattgctacaggcatcgtggtgtcacgctcgtcgtttggtatggcttcattcagctc
cggttcccaacgatcaaggcgagttacatgatcccccatgttgtgcaaaaaagoggttagct
ccttcggtcctccgatcgttgtcagaagtaagttggccgcagtgttatcactcatggttatg
gcagcactgcataattctcttactgtcatgccatcogtaagatgcttttctgtgactggtga
gtactcaaccaagtcattctgagaatagtgtatgcggcgaccgagttgctcttgcccggcgt
caatacgggataataccgcgccacatagcagaactttaaaagtgctcatcattggaaaacgt
tcttcggggcgaaaactctcaaggatcttaccgctgttgagatccagttcgatgtaacccac
tcgtgcacccaactgatcttcagcatcttttactttcaccagcgtttctgggtgagcaaaaa
caggaaggcaaaatgccgcaaaaaagggaataagggcgacacggaaatgttgaatactcata
ctcttcctttttcaatattattgaagcatttatcagggttattgtctcatgagcggatacat
atttgaatgtatttagaaaaataaacaaataggggttccgcgcacatttccccgaaaagtgc
cac
SEQ ID NO: 84
pJRH1 (SaCas9 used for all JCR179/183 projects, SaCas9
is in uppercase; NLS is lowercase, boided, and underlined)
ctaaattgtaagcgttaatattttgttaaaattcgcgttaaatttttgttaaatcagctcat
tttttaaccaataggccgaaatcggcaaaatcccttataaatcaaaagaatagaccgagata
gggttgagtgttgttccagtttggaacaagagtccactattaaagaacgtggactccaacgt
caaagggcgaaaaaccgtctatcagggcgatggcccactacgtgaaccatcaccctaatcaa
gttttttggggtcgaggtgccgtaaagcactaaatcggaaccctaaagggagcccccgattt
agagcttgacggggaaagccggcgaacgtggcgagaaaggaagggaagaaagcgaaaggagc
gggcgctagggcgctggcaagtgtagcggtcacgctgcgcgtaaccaccacacccgccgcgc
ttaatgcgccgctacagggcgcgtcccattcgccattcaggctgcgcaactgttgggaaggg
cgatcggtgcgggcctcttcgctattacgccagctggcgaaagggggatgtgctgcaaggcg
attaagttgggtaacgccagggttttcccagtcacgacgttgtaaaacgacggccagtgagc
gcgcgtaatacgactcactatagggcgaattgggtacctttaattctagtactatgcatgcg
ttgacattgattattgactagttattaatagtaatcaattacggggtcattagttcatagcc
catatatggagttccgcgttacataacttacggtaaatggcccgcctggctgaccgcccaac
gacccccgcccattgacgtcaataatgacgtatgttcccatagtaacgccaatagggacttt
ccattgacgtcaatgggtggagtatttacggtaaactgcccacttggcagtacatcaagtgt
atcatatgccaagtacgccccctattgacgtcaatgacggtaaatggcccgcctggcattat
gcccagtacatgaccttatgggactttcctacttggcagtacatctacgtattagtcatcgc
tattaccatggtgatgcggttttggcagtacatcaatgggcgtggatagcggtttgactcac
ggggatttccaagtctccaccccattgacgtcaatgggagtttgttttggcaccaaaatcaa
cgggactttccaaaatgtcgtaacaactccgccccattgacgcaaatgggcggtaggcgtgt
acggtgggaggtctatataagcagagctctctggctaactaccggtgccaccatggccccaa
agaagaagcggaaggtcggtatccacggagtcccagcagcCAAGCGGAACTACATCCTGGGC
CTGGACATCGGCATCACCAGCGTGGGCTACGGCATCATCGACTACGAGACACGGGACGTGAT
CGATGCCGGCGTGCGGCTGTTCAAAGAGGCCAACGTGGAAAACAACGAGGGCAGGCGGAGCA
AGAGAGGCGCCAGAAGGCTGAAGCGGCGGAGGCGGCATAGAATCCAGAGAGTGAAGAAGCTG
CTGTTCGACTACAACCTGCTGACCGACCACAGCGAGCTGAGCGGCATCAACCCCTACGAGGC
CAGAGTGAAGGGCCTGAGCCAGAAGCTGAGCGAGGAAGAGTTCTCTGCCGCCCTGCTGCACC
TGGCCAAGAGAAGAGGCGTGCACAACGTGAACGAGGTGGAAGAGGACACCGGCAACGAGCTG
TCCACCAAAGAGCAGATCAGCCGGAACAGCAAGGCCCTGGAAGAGAAATACGTGGCCGAACT
GCAGCTGGAACGGCTGAAGAAAGACGGCGAAGTGCGGGGCAGCATCAACAGATTCAAGACCA
GCGACTACGTGAAAGAAGCCAAACAGCTGCTGAAGGTGCAGAAGGCCTACCACCAGCTGGAC
CAGAGCTTCATCGACACCTACATCGACCTGCTGGAAACCCGGCGGACCTACTATGAGGGACC
TGGCGAGGGCAGCCCCTTCGGCTGGAAGGACATCAAAGAATGGTACGAGATGCTGATGGGCC
ACTGCACCTACTTCCCCGAGGAACTGCGGAGCGTGAAGTACGCCTACAACGCCGACCTGTAC
AACGCCCTGAACGACCTGAACAATCTCGTGATCACCAGGGACGAGAACGAGAAGCTGGAATA
TTACGAGAAGTTCCAGATCATCGAGAACGTGTTCAAGCAGAAGAAGAAGCCCACCCTGAAGC
AGATCGCCAAAGAAATCCTCGTGAACGAAGAGGATATTAAGGGCTACAGAGTGACCAGCACC
GGCAAGCCCGAGTTCACCAACCTGAAGGTGTACCACGACATCAAGGACATTACCGCCCGGAA
AGAGATTATTGAGAACGCCGAGCTGCTGGATCAGATTGCCAAGATCCTGACCATCTACCAGA
GCAGCGAGGACATCCAGGAAGAACTGACCAATCTGAACTCCGAGCTGACCCAGGAAGAGATC
GAGCAGATCTCTAATCTGAAGGGCTATACCGGCACCCACAACCTGAGCCTGAAGGCCATCAA
CCTGATCCTGGACGAGCTGTGGCACACCAACGACAACCAGATCGCTATCTTCAACCGGCTGA
AGCTGGTGCCCAAGAAGGTGGACCTGTCCCAGCAGAAAGAGATCCCCACCACCCTGGTGGAC
GACTTCATCCTGAGCCCCGTCGTGAAGAGAAGCTTCATCCAGAGCATCAAAGTGATCAACGC
CATCATCAAGAAGTACGGCCTGCCCAACGACATCATTATCGAGCTGGCCCGCGAGAAGAACT
CCAAGGACGCCCAGAAAATGATCAACGAGATGCAGAAGCGGAACCGGCAGACCAACGAGCGG
ATCGAGGAAATCATCCGGACCACCGGCAAAGAGAACGCCAAGTACCTGATCGAGAAGATCAA
GCTGCACGACATGCAGGAAGGCAAGTGCCTGTACAGCCTGGAAGCCATCCCTCTGGAAGATC
TGCTGAACAACCCCTTCAACTATGAGGTGGACCACATCATCCCCAGAAGCGTGTCCTTCGAC
AACAGCTTCAACAACAAGGTGCTCGTGAAGCAGGAAGAAAACAGCAAGAAGGGCAACCGGAC
CCCATTCCAGTACCTGAGCAGCAGCGACAGCAAGATCAGCTACGAAACCTTCAAGAAGCACA
TCCTGAATCTGGCCAAGGGCAAGGGCAGAATCAGCAAGACCAAGAAAGAGTATCTGCTGGAA
GAACGGGACATCAACAGGTTCTCCGTGCAGAAAGACTTCATCAACCGGAACCTGGTGGATAC
CAGATACGCCACCAGAGGCCTGATGAACCTGCTGCGGAGCTACTTCAGAGTGAACAACCTGG
ACGTGAAAGTGAAGTCCATCAAAGGCGGCTTCACCAGCTTTCTGCGGCGGAAGTGGAAGTTT
AAGAAAGAGCGGAACAAGGGGTACAAGCACCACGCCGAGGACGCCCTGATCATTGCCAACGC
CGATTTCATCTTCAAAGAGTGGAAGAAACTGGACAAGGCCAAAAAAGTGATGGAAAACCAGA
TGTTCGAGGAAAAGCAGGCCGAGAGCATGCCCGAGATCGAAACCGAGCAGGAGTACAAAGAG
ATCTTCATCACCCCCCACCAGATCAAGCACATTAAGGACTTCAAGGACTACAAGTACAGCCA
CCGGGTGGACAAGAAGCCTAATAGAGAGCTGATTAACGACACCCTGTACTCCACCCGGAAGG
ACGACAAGGGCAACACCCTGATCGTGAACAATCTGAACGGCCTGTACGACAAGGACAATGAC
AAGCTGAAAAAGCTGATCAACAAGAGCCCCGAAAAGCTGCTGATGTACCACCACGACCCCCA
GACCTACCAGAAACTGAAGCTGATTATGGAACAGTACGGCGACGAGAAGAATCCCCTGTACA
AGTACTACGAGGAAACCGGGAACTACCTGACCAAGTACTCCAAAAAGGACAACGGCCCCGTG
ATCAAGAAGATTAAGTATTACGGCAACAAACTGAACGCCCATCTGGACATCACCGAGGACTA
CCCCAACAGCAGAAACAAGGTCGTGAAGCTGTCCCTGAAGCCCTACAGATTCGACGTGTACC
TGGACAATGGCGTGTACAAGTTCGTGACCGTGAAGAATCTGGATGTGATCAAAAAAGAAAAC
TACTACGAAGTGAATAGCAAGTGCTATGAGGAAGCTAAGAAGCTGAAGAAGATCAGCAACCA
GGCCGAGTTTATCGCCTCCTTCTACAACAACGATCTGATCAAGATCAACGGCGAGCTGTATA
GAGTGATCGGCGTGAACAACGACCTGCTGAACCGGATCGAAGTGAACATGATCGACATCACC
TACCGCGAGTACCTGGAAAACATGAACGACAAGAGGCCCCCCAGGATCATTAAGACAATCGC
CTCCAAGACCCAGAGCATTAAGAAGTACAGCACAGACATTCTGGGCAACCTGTATGAAGTGA
AATCTAAGAAGCACCCTCAGATCATCAAAAAGGGCaaaaggccggcggccacgaaaaaggcc
ggccaggcaaaaaagaaaaagggatCctacccatacgatgttccagattacgcttaagaatt
cctagagctcgctgatcagcctcgactgtgccttctagttgccagccatctgttgtttgccc
ctcccccgtgccttccttgaccctggaaggtgccactcccactgtcctttcctaataaaatg
aggaaattgcatcgcattgtctgagtaggtgtcattctattctggggggtggggtggggcag
gacagcaagggggaggattgggaagagaatagcaggcatgctggggaggtagcggccgcccg
cggtggagctccagcttttgttccctttagtgagggttaattgcgcgcttggcgtaatcatg
gtcatagctgtttcctgtgtgaaattgttatccgctcacaattccacacaacatacgagccg
gaagcataaagtgtaaagcctggggtgcctaatgagtgagctaactcacattaattgcgttg
cgctcactgcccgctttccagtcgggaaacctgtcgtgccagctgcattaatgaatcggcca
acgcgcggggagaggcggtttgcgtattgggcgctcttccgcttcctcgctcactgactcgc
tgcgctcggtcgttcggctgcggcgagcggtatcagctcactcaaaggcggtaatacggtta
tccacagaatcaggggataacgcaggaaagaacatgtgagcaaaaggccagcaaaaggccag
gaaccgtaaaaaggccgcgttgctggcgtttttccataggctccgcccccctgacgagcatc
acaaaaatcgacgctcaagtcagaggtggcgaaacccgacaggactataaagataccaggcg
tttccccctggaagctccctCgtgcgctctcctgttccgaccctgccgcttaccggatacct
gtccgcctttctcccttcgggaagcgtggcgctttctcatagctcacgctgtaggtatctca
gttcggtgtaggtcgttcgctccaagctgggctgtgtgcacgaaccccccgttcagcccgac
cgctgcgccttatccggtaactatcgtcttgagtccaacccggtaagacacgacttatcgcc
actggcagcagccactggtaacaggattagcagagcgaggtatgtaggcggtgctacagagt
tcttgaagtggtggcctaactacggctacactagaaggacagtatttggtatctgcgctctg
ctgaagccagttaccttcggaaaaagagttggtagctcttgatccggcaaacaaaccaccgc
tggtagcggtggtttttttgtttgcaagcagcagattacgcgcagaaaaaaaggatctcaag
aagatcctttgatcttttctacggggtctgacgctcagtggaacgaaaactcacgttaaggg
attttggtcatgagattatcaaaaaggatcttcacctagatccttttaaattaaaaatgaag
ttttaaatcaatctaaagtatatatgagtaaacttggtctgacagttaccaatgcttaatca
gtgaggcacctatctcagcgatctgtctatttcgttcatccatagttgcctgactccccgtc
gtgtagataactacgatacgggagggcttaccatctggccccagtgctgcaatgataccgcg
agacccacgctcaccggctccagatttatcagcaataaaccagccagccggaagggccgagc
gcagaagtggtcctgcaactttatccgcctccatccagtctattaattgttgccgggaagct
agagtaagtagttcgccagttaatagtttgcgcaacgttgttgccattgctacaggcatcgt
ggtgtcacgctcgtcgtttggtatggcttcattcagctccggttcccaacgatcaaggcgag
ttacatgatcccccatgttgtgcaaaaaagcggttagctccttcggtcctccgatcgttgtc
agaagtaagttggccgcagtgttatcactcatggttatggcagcactgcataattctcttac
tgtcatgccatccgtaagatgcttttctgtgactggtgagtactcaaccaagtcattctgag
aatagtgtatgcggcgaccgagttgctcttgcccggcgtcaatacgggataataccgcgcca
catagcagaactttaaaagtgctcatcattggaaaacgttcttcggggcgaaaactctcaag
gatcttaccgctgttgagatccagttcgatgtaacccactcgtgcacccaactgatcttcag
catcttttactttcaccagcgtttctgggtgagcaaaaacaggaaggcaaaatgccgcaaaa
aagggaataagggcgacacggaaatgttgaatactcatactcttcctttttcaatattattg
aagcatttatcagggttattgtctcatgagcggatacatatttgaatgtatttagaaaaata
aacaaataggggttccgcgcacatttccccgaaaagtgccac
SEQ ID NO: 85
NLS sequence in PT366
aaaaggccggcggccacgaaaaaggccggccaggcaaaaaagaaaaag
SEQ ID NO: 86
pDO203-Generic backbone to clone gRNAs into for SpCas9;
JCR94 and JCR99 were put into the site in bold to test
in cells then to make mRNA from for making the hDMD-
delta52/mdx mouse
ctaaattgtaagcgttaatattttgttaaaattcgcgttaaatttttgttaaatcagctcat
tttttaaccaataggccgaaatcggcaaaatcccttataaatcaaaagaatagaccgagata
gggttgagtgttgttccagtttggaacaagagtccactattaaagaacgtggactccaacgt
caaagggcgaaaaaccgtctatcagggcgatggcccactacgtgaaccatcaccctaatcaa
gttttttggggtcgaggtgccgtaaagcactaaatcggaaccctaaagggagcccccgattt
agagcttgacggggaaagccggcgaacgtggcgagaaaggaagggaagaaagcgaaaggagc
gggcgctagggcgctggcaagtgtagcggtcacgctgcgcgtaaccaccacacccgccgcgc
ttaatgcgccgctacagggcgcgtcccattcgccattcaggctgcgcaactgttgggaaggg
cgatcggtgcgggcctcttcgctattacgccagctggcgaaagggggatgtgctgcaaggcg
attaagttgggtaacgccagggttttcccagtcacgacgttgtaaaacgacggccagtgagc
gcgcgtaatacgactcactatagggcgaattgggtaccgagggcctatttcccatgattcct
tcatatttgcatatacgatacaaggctgttagagagataattggaattaatttgactgtaaa
cacaaagatattagtacaaaatacgtgacgtagaaagtaataatttcttgggtagtttgcag
ttttaaaattatgttttaaaatggactatcatatgcttatcgtaacttgaaagtatttcgat
ttcttggctttatatatcttgtggaaaggacgaaacaccGGGTCTTCGAGAAGACCTgtttt
agagctagaaatagcaagttaaaataaggctagtccgttatcaacttgaaaaagtggcaccg
agtcggtgctttttttccgcggtggagctccagcttttgttccctttagtgagggttaattg
cgcgcttggcgtaatcatggtcatagctgtttcctgtgtgaaattgttatccgctcacaatt
ccacacaacatacgagccggaagcataaagtgtaaagcctggggtgcctaatgagtgagcta
actcacattaattgcgttgcgctcactgcccgctttccagtcgggaaacctgtcgtgccagc
tgcattaatgaatcggccaacgcgcggggagaggcggtttgcgtattgggcgctcttccgct
tcctcgctcactgactcgctgcgctcggtcgttcggctgcggcgagcggtatcagctcactc
aaaggcggtaatacggttatccacagaatcaggggataacgcaggaaagaacatgtgagcaa
aaggccagcaaaaggccaggaaccgtaaaaaggccgcgttgctggcgtttttccataggctc
cgcccccctgacgagcatcacaaaaatcgacgctcaagtcagaggtggcgaaacccgacagg
actataaagataccaggcgtttccccctggaagctccctcgtgcgctctcctgttccgaccc
tgccgcttaccggatacctgtccgcctttctcccttcgggaagcgtggcgctttctcatagc
tcacgctgtaggtatctcagttcggtgtaggtcgttcgctccaagctgggctgtgtgcacga
accccccgttcagcccgaccgctgcgccttatccggtaactatcgtcttgagtccaacccgg
taagacacgacttatcgccactggcagcagccactggtaacaggattagcagagcgaggtat
gtaggcggtgctacagagttcttgaagtggtggcctaactacggctacactagaaggacagt
atttggtatctgcgctctgctgaagccagttaccttcggaaaaagagttggtagctcttgat
ccggcaaacaaaccaccgctggtagcggtggtttttttgtttgcaagcagcagattacgcgc
agaaaaaaaggatctcaagaagatcctttgatcttttctacggggtctgacgctcagtggaa
cgaaaactcacgttaagggattttggtcatgagattatcaaaaaggatcttcacctagatcc
ttttaaattaaaaatgaagttttaaatcaatctaaagtatatatgagtaaacttggtctgac
agttaccaatgcttaatcagtgaggcacctatctcagcgatctgtctatttcgttcatccat
agttgcctgactccccgtcgtgtagataactacgatacgggagggcttaccatctggcccca
gtgctgcaatgataccgcgagacccacgctcaccggctccagatttatcagcaataaaccag
ccagccggaagggccgagcgcagaagtggtcctgcaactttatccgcctccatccagtctat
taattgttgccgggaagctagagtaagtagttcgccagttaatagtttgcgcaacgttgttg
ccattgctacaggcatcgtggtgtcacgctcgtcgtttggtatggcttcattcagctccggt
tcccaacgatcaaggcgagttacatgatcccccatgttgtgcaaaaaagcggttagctcctt
cggtcctccgatcgttgtcagaagtaagttggccgcagtgttatcactcatggttatggcag
cactgcataattctcttactgtcatgccatccgtaagatgcttttctgtgactggtgagtac
tcaaccaagtcattctgagaatagtgtatgcggcgaccgagttgctcttgcccggcgtcaat
acgggataataccgcgccacatagcagaactttaaaagtgctcatcattggaaaacgttctt
cggggcgaaaactctcaaggatcttaccgctgttgagatccagttcgatgtaacccactcgt
gcacccaactgatcttcagcatcttttactttcaccagcgtttctgggtgagcaaaaacagg
aaggcaaaatgccgcaaaaaagggaataagggcgacacggaaatgttgaatactcatactct
tcctttttcaatattattgaagcatttatcagggttattgtctcatgagcggatacatattt
gaatgtatttagaaaaataaacaaataggggttccgcgcacatttccccgaaaagtgccac

| | | |
|---|---|---|
| SEQ ID NO: 87 | JRH261 | ctccggaatgtctccatttg |
| SEQ ID NO: 88 | JRH264 | atgagggagagactggcatc |
| SEQ ID NO: 89 | exon 48 primer (forward) | gtttccagagctttacctgagaa |
| SEQ ID NO: 90 | exon 52 primer (reverse) | CTTTTATGAATGCTTCTCCAAG |
| SEQ ID NO: 91 | JCR 89 forward | aagttacttgtccaggcatga |
| SEQ ID NO: 92 | JCR 89 reverse | gaaaaacttctgccaacttttatca |
| SEQ ID NO: 93 | JCR 91 forward | tgcaaataacaaaagtagccataca |
| SEQ ID NO: 94 | JCR 91 reverse | tctttagaaaggcttgaaagctg |
| SEQ ID NO: 95 | forward primer | cttcactgctggccagttta |
| SEQ ID NO: 96 | reverse primer PCR | ttgaacatggcattgcataaA |
| SEQ ID NO: 97 | JCR160 forward primer | cgggcttggacagaacttac |
| SEQ ID NO: 98 | JCR160 reverse primer | ctgcgtagtgccaaaacaaa |
| SEQ ID NO: 99 | JCR 157 forward primer | gagatgtcttttgcagctttcc |
| SEQ ID NO: 100 | JCR157 reverse primer | gggaccttggtaaagccaca |
| SEQ ID NO: 101 | forward primer | tgcctttcaatcattgtttcg |
| SEQ ID NO: 102 | reverse primer | agaaggcaaattggcacaga |
| SEQ ID NO: 103 | JCR179 reverse primer | aaggccccaaaatgtgaaat |
| SEQ ID NO: 104 | JCR183 forward primer | gagtttggctcaaattgttactctt |
| SEQ ID NO: 105 | forward primer | tggcggcgttttcattat |
| SEQ ID NO: 106 | reverse primer | TTCGATCCGTAATGATTGTTCTAGCC |
| SEQ ID NO: 107 | upstream reverse primer | TTGTGTGTCCCATGCTTGTT |
| SEQ ID NO: 108 | downstream forward | CAACGCTGAAGAACCCTGAT |
| | primer | |
| SEQ ID NO: 109 | reverse primer for | tttctgtgattttcttttggattg |
| | seguencing | |
| SEQ ID NO: 110 | exemplary first gRNA | GATTGGCTTTGATTTCCCTA |
| SEQ ID NO: 111 | exemplary second gRNA | GCAGTTGCCTAAGAACTGGT |
| SEQ ID NO: 112 | JCR89 | aaaGAUAUAUAAUGUCAUGAAU |
| SEQ ID NO: 125 | JCR91 | gCaGAAUCAAAUAUAAUAGUCU |
| SEQ ID NO: 113 | JCR159 | CAAUUAAAUUUGACUUAUUGUU |
| SEQ ID NO: 126 | JCR160 | CUAGACCAUUUCCCACCAGUUC |
| SEQ ID NO: 127 | JCR167 | AGGACUUUUAUUUACCAAAGGA |
| SEQ ID NO: 128 | JCR166 | AUCCAAGUCCAUUUGAUUCCUA |
| SEQ ID NO: 114 | JCR168 | UAAUUCUUUCUAGAAAGAGCCU |
| SEQ ID NO: 115 | JCR170 | GGACAUGUGCAAGAUGCAAGAG |
| SEQ ID NO: 116 | JCR171 | UGUAUGUAGAAGACCUCUAAGU |
| SEQ ID NO: 117 | JCR156 | UCCCCUCACCACUCACCUCUGA |
| SEQ ID NO: 118 | JCR157 | CUCUGAUAACCCAGCUGUGUGU |
| SEQ ID NO: 119 | JCR176 | cucugauaACCCAGcugug |
| SEQ ID NO: 120 | JCR177 | cucugauaACCCAGcugugu |
| SEQ ID NO: 121 | JCR178 | cucugauaACCCAGcugugug |
| SEQ ID NO: 122 | JCR179 | cucugauaACCCAGcuguguguu |
| SEQ ID NO: 129 | JCR180 | Cuagaccauuucccaccag |
| SEQ ID NO: 130 | JCR181 | cuagaccauuucccaccaga |
| SEQ ID NO: 131 | JCR182 | cuagaccauaucccaccaguu |
| SEQ ID NO: 132 | JCR183 | caagaCcauuacccaccaguucu |
| SEQ ID NO: 123 | JCR94 | aacaaauaucccuuagaaac |
| SEQ ID NO: 133 | JCR99 | aaaguaauaaacaauacaa |
| SEQ ID NO: 124 | exemplary first gRNA | GAUUGGCUUUGAUUUCCCUA |
| SEQ ID NO: 134 | exemplary second gRNA | GCAGUUGCCUAAGAACUGGU |

1. A lipid nanoparticle or microparticle for delivering a DNA targeting system to a muscle cell, the DNA targeting system comprising:
at least one gRNA molecule targeting a fragment of a mutant dystrophin gene; and/or
a polynucleotide encoding a Cas9 nuclease.

2. The lipid nanoparticle or microparticle of claim 1, wherein the at least one gRNA molecule comprises a first gRNA molecule and a second gRNA molecule.

3. The lipid nanoparticle or microparticle of claim 1 or 2, wherein the polynucleotide encoding a Cas9 nuclease is mRNA.

4. The lipid nanoparticle or microparticle of any one of claims 2-3, wherein the first gRNA molecule and the second gRNA molecule each comprise a targeting domain, wherein the first gRNA molecule is encoded by a polynucleotide comprising a nucleotide sequence selected from SEQ ID NO: 1, SEQ ID NO: 3, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 37, SEQ ID NO: 41, SEQ ID NO: 83, or SEQ ID NO: 110 or a fragment or complement thereof or comprises a nucleotide sequence selected from SEQ ID NOs: 112-124 or a fragment or complement thereof, wherein the second gRNA molecule is encoded by a polynucleotide comprising a nucleotide sequence selected from SEQ ID NO: 2, SEQ ID NO: 4, SEQ ID NO: 5, SEQ ID NO: 6, SEQ ID NO: 16, SEQ ID NO: 17, SEQ ID NO: 18, SEQ ID NO: 19, SEQ ID NO: 38, SEQ ID NO: 42, SEQ ID NO: 84, or SEQ ID NO: 111 or a fragment or complement thereof or comprises a nucleotide sequence selected from SEQ ID NOs: 125-134 or a fragment or complement thereof, and wherein the first gRNA molecule and the second gRNA molecule comprise different targeting domains.

5. The lipid nanoparticle or microparticle of claim 4, wherein the first gRNA molecule comprises a targeting domain comprising the nucleotide sequence of SEQ ID NO: 110 or a fragment or complement thereof or comprises the nucleotide sequence of SEQ ID NO: 124 or a fragment or complement thereof, and wherein the second gRNA molecule comprises a targeting domain comprising the nucleotide sequence of SEQ ID NO: 111 or a fragment or complement thereof or comprises the nucleotide sequence of SEQ ID NO: 134 or a fragment or complement thereof.

6. The lipid nanoparticle or microparticle of any one of claims 1-5, wherein the at least one gRNA and the polynucleotide encoding the Cas9 nuclease are encapsulated in the same lipid nanoparticle or microparticle.

7. The lipid nanoparticle or microparticle of claim any one of claims 1-5, wherein the at least one gRNA and the polynucleotide encoding the Cas9 nuclease are each encapsulated in a separate lipid nanoparticle.

8. The lipid nanoparticle or microparticle of any one of claims 1-7, wherein the lipid nanoparticle or microparticle is selected from the group consisting of solid lipid nanoparticle (SLN), nanostructured lipid carrier (NLC), lipid-drug conjugate (LDC) nanoparticle, lipid nanocapsule (LNC), polymer lipid hybrid nanoparticle (PLN), and solid lipid microparticle (SLM).

9. The lipid nanoparticle or microparticle of claim 8, wherein the lipid nanoparticle or microparticle is a solid lipid nanoparticle (SLN).

10. The lipid nanoparticle or microparticle of claim 8, wherein the lipid nanoparticle or microparticle is a nanostructured lipid carrier (NLC).

11. The lipid nanoparticle or microparticle of claim 8, wherein the lipid nanoparticle or microparticle is a lipid-drug conjugate (LDC) nanoparticle.

12. The lipid nanoparticle or microparticle of claim 8, wherein the lipid nanoparticle or microparticle is a lipid nanocapsule (LNC).

13. The lipid nanoparticle or microparticle of claim 8, wherein the lipid nanoparticle or microparticle is a polymer lipid hybrid nanoparticle (PLN).

14. The lipid nanoparticle or microparticle of claim 8, wherein the lipid nanoparticle or microparticle is a solid lipid microparticle (SLM).

15. The lipid nanoparticle or microparticle of any one of claims 1-14, wherein the at least one gRNA molecule targets an exon selected from exons 1-8, 10, 11, 12, 14, 16-22, 43-59, and 61-86 of the mutant dystrophin gene, or an intron that flanks an exon selected from exons 1-8, 10, 11, 12, 14, 16-22, 43-59, and 61-66 of the mutant dystrophin gene.

16. The lipid nanoparticle or microparticle of any one of claims 1-15, wherein the DNA targeting system further comprises a donor sequence that comprises an exon of the wild-type dystrophin gene or a functional equivalent thereof, and wherein the exon is selected from exons 1-8, 10, 11, 12, 14, 16-22, 43-59, and 61-66 of the wild-type dystrophin gene.

17. The lipid nanoparticle or microparticle of any one of claims 1-16, wherein the at least one gRNA molecule targets two introns that flank exon 51 of a human dystrophin gene.

18. The lipid nanoparticle or microparticle of any one of claims 1-17, wherein the DNA targeting system induces a first double strand break in a first intron flanking exon 51 of a human dystrophin gene and a second double strand break in a second intron flanking exon 51 of a human dystrophin gene.

19. The lipid nanoparticle or microparticle of any one of claims 1-18, wherein the polynucleotide encodes SpCas9 or SaCas9.

20. The lipid nanoparticle or microparticle of any one of claims 3-19, wherein the mRNA is a modified mRNA.

21. The lipid nanoparticle or microparticle of claim 20, wherein the modified mRNA comprises one or more modifications selected from an N terminal NLS, a C terminal NLS, an HA Tag, and a uridine substitution.

22. The lipid nanoparticle or microparticle of any one of claims 1-21, wherein the muscle cell is selected from a skeletal muscle cell, a cardiac muscle cell, and a smooth muscle cell.

23. A composition comprising the lipid nanoparticle or microparticle of any one of claims 1-22 and a pharmaceutically acceptable carrier.

24. A method of treating Duchenne Muscular Dystrophy in a subject, the method comprising administering to the subject the lipid nanoparticle or microparticle of any one of claims 1-22 or the composition of claim 23.

25. The method of claim 24, wherein the subject experiences no or a limited humoral response that is cross reactive to the Cas9 nuclease after administration.

26. The method of claim 24 or 25, where the subject comprises a mutant dystrophin gene.

27. A method of genome editing a mutant dystrophin gene in a subject, the method comprising administering to the subject the lipid nanoparticle or microparticle of any one of claims 1-22 or the composition of claim 23.

28. The method of any one of claims 28-27, wherein the mutant dystrophin gene comprises a premature stop codon, a disrupted reading frame, an aberrant splice acceptor site, or an aberrant splice donor site, or a combination thereof.

29. The method of any one of claims 28-27, wherein the mutant dystrophin gene comprises a frameshift mutation that causes a premature stop codon and a truncated gene product.

30. The method of any one of claims 28-27, wherein the mutant dystrophin gene comprises a deletion of one or more exons that disrupts the reading frame.

31. The method of claim 27, wherein genome editing of the mutant dystrophin gene comprises a deletion of a premature stop codon, correction of a disrupted reading frame, modulation of splicing by disruption of a splice acceptor site, modulation of splicing by disruption of a splice donor sequence, deletion of exon 51, or a combination thereof.

32. The method of any one of claims 27-31, wherein the mutant dystrophin gene is edited by homology-directed repair.

33. The method of any one of claims 24-32, wherein dystrophin expression in the subject is increased by at least 1%, 2%, 5%, 10%, 15%, 20%, 25%, 30%, 35%, 40%, 45%, or at least 50% after editing.

34. The method of any one of claims 24-33, wherein the lipid nanoparticle or microparticle is administered to the subject before birth or within 1-2 days of birth.

35. The method of any one of claims 24-34, wherein the lipid nanoparticle or microparticle is administered to the subject intramuscularly, intravenously, or a combination thereof.

36. The method of any one of claims 24-35, wherein administration of the lipid nanoparticle or the microparticle or the compositions leads to expression of a functional or partially-functional dystrophin protein in the subject.

37. A kit comprising the lipid nanoparticle or microparticle of any one of claims 1-22.


SEQUENCE LISTING (134 sequences)

SEQ ID NO: 1 - DNA, 22 bp, Artificial Sequence
  Synthetic
aaagatatataatgtcatgaat

SEQ ID NO: 2 - DNA, 22 bp, Artificial Sequence
  Synthetic
gcagaatcaaatataatagtct

SEQ ID NO: 3 - DNA, 22 bp, Artificial Sequence
  Synthetic
caattaaatttgacttattgtt

SEQ ID NO: 4 - DNA, 22 bp, Artificial Sequence
  Synthetic
ctagaccatttcccaccagttc

SEQ ID NO: 5 - DNA, 22 bp, Artificial Sequence
  Synthetic
aggacttttatttaccaaagga

SEQ ID NO: 6 - DNA, 22 bp, Artificial Sequence
  Synthetic
atccaagtccatttgattccta

SEQ ID NO: 7 - DNA, 22 bp, Artificial Sequence
  Synthetic
taattctttctagaaagagcct

SEQ ID NO: 8 - DNA, 22 bp, Artificial Sequence
  Synthetic
ggacatgtgcaagatgcaagag

SEQ ID NO: 9 - DNA, 22 bp, Artificial Sequence
  Synthetic
tgtatgtagaagacctctaagt

SEQ ID NO: 10 - DNA, 22 bp, Artificial Sequence
  Synthetic
tcccctcaccactcacctctga

SEQ ID NO: 11 - DNA, 22 bp, Artificial Sequence
  Synthetic
ctctgataacccagctgtgtgt

SEQ ID NO: 12 - DNA, 19 bp, Artificial Sequence
  Synthetic
ctctgataacccagctgtg

SEQ ID NO: 13 - DNA, 20 bp, Artificial Sequence
  Synthetic
ctctgataacccagctgtgt

SEQ ID NO: 14 - DNA, 21 bp, Artificial Sequence
  Synthetic
ctctgataacccagctgtgtg

SEQ ID NO: 15 - DNA, 23 bp, Artificial Sequence
  Synthetic
ctctgataacccagctgtgtgtt

SEQ ID NO: 16 - DNA, 19 bp, Artificial Sequence
  Synthetic
ctagaccatttcccaccag

SEQ ID NO: 17 - DNA, 20 bp, Artificial Sequence
  Synthetic
ctagaccatttcccaccagt

SEQ ID NO: 18 - DNA, 21 bp, Artificial Sequence
  Synthetic
ctagaccatttcccaccagtt

SEQ ID NO: 19 - DNA, 23 bp, Artificial Sequence
  Synthetic
ctagaccatttcccaccagttct

SEQ ID NO: 20 - DNA, 7 bp, Homo sapiens
  misc_feature (1)..(2): n is a, c, g, or t
  misc_feature (7)..(7): w is a, or t
nnagaaw

SEQ ID NO: 21 - DNA, 4 bp, Homo sapiens
  misc_feature (1)..(1): n is a, c, g, or t
  misc_feature (4)..(4): r is a, or g
naar

SEQ ID NO: 22 - DNA, 5 bp, Homo sapiens
  misc_feature (1)..(2): n is a, c, g, or t
  misc_feature (4)..(5): r is a, or g
nngrr

SEQ ID NO: 23 - DNA, 6 bp, Homo sapiens
  misc_feature (1)..(2): n is a, c, g, or t
  misc_feature (4)..(5): r is a, or g
  misc_feature (6)..(6): n is a, c, g, or t
nngrrn

SEQ ID NO: 24 - DNA, 6 bp, Homo sapiens
  misc_feature (1)..(2): n is a, c, g, or t
  misc_feature (4)..(5): r is a, or g
nngrrt

SEQ ID NO: 25 - DNA, 6 bp, Homo sapiens
  misc_feature (1)..(2): n is a, c, g, or t
  misc_feature (4)..(5): r is a, or g
nngrrv

SEQ ID NO: 26 - DNA, 4107 bp, Artificial Sequence
  Synthetic
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 27 - protein, 1368 aa, Streptococcus pyogenes [sequence omitted]

SEQ ID NO: 28 - DNA, 3158 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 29 - DNA, 3159 bp, Artificial Sequence
  Synthetic
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 30 - DNA, 3159 bp, Artificial Sequence
  Synthetic
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 31 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 32 - DNA, 3242 bp, Artificial Sequence
  Synthetic
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaattc

SEQ ID NO: 33 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 34 - DNA, 3159 bp, Artificial Sequence
  Synthetic
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 35 - DNA, 3159 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 36 - DNA, 5 bp, Homo sapiens
  misc_feature (1)..(1): n is a, c, g, or t
  misc_feature (4)..(4): n is a, c, g, or t
nggng

SEQ ID NO: 37 - DNA, 3238 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaacacacagctgggttatcagaggttttagtactctggaaacagaatctactaaaacaaggcaaaatgccgtgtttatctcgtcaacttgttggcgagatttttttgcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 38 - DNA, 3238 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccagaactggtgggaaatggtctaggttttagtactctggaaacagaatctactaaaacaaggcaaaatgccgtgtttatctcgtcaacttgttggcgagatttttttgcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 39 - DNA, 7450 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaacacacagctgggttatcagaggttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 40 - DNA, 7450 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagaactggtgggaaatggtctaggttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 41 - DNA, 20 bp, Artificial Sequence
  Synthetic
aacaaatatcccttagtatc

SEQ ID NO: 42 - DNA, 20 bp, Artificial Sequence
  Synthetic
aatgtatttcttctattcaa

SEQ ID NO: 43 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 44 - DNA, 3156 bp, Artificial Sequence
  Synthetic
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 45 - protein, 1052 aa, Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 46 - DNA, 52 bp, Artificial Sequence
  Synthetic
aacgctgaagaaccctgataatagaagaaatacatttttaaatcaattcagg

SEQ ID NO: 47 - DNA, 51 bp, Artificial Sequence
  Synthetic
aagaaccctgatattatcttagtagattaatagaagaaatacatttttaaa

SEQ ID NO: 48 - DNA, 42 bp, Artificial Sequence
  Synthetic
gctgaagaaccctgaaaaaatacatttttttatcaattcagg

SEQ ID NO: 49 - DNA, 36 bp, Artificial Sequence
  Synthetic
gaatgattttcttgtagaagaaataacaattaaatc

SEQ ID NO: 50 - DNA, 52 bp, Artificial Sequence; identical to SEQ ID NO: 46
  Synthetic

SEQ ID NO: 51 - DNA, 59 bp, Artificial Sequence
  Synthetic
aacgctgaagaaccctgatattatcttagtagattaatagaagaaatacatttttaaat

SEQ ID NO: 52 - DNA, 59 bp, Artificial Sequence; identical to SEQ ID NO: 51
  Synthetic

SEQ ID NO: 53 - DNA, 6 bp, Artificial Sequence
  Synthetic
aagagt

SEQ ID NO: 54 - DNA, 6 bp, Artificial Sequence
  Synthetic
gggaat

SEQ ID NO: 55 - DNA, 3 bp, Artificial Sequence
  Synthetic
agg

SEQ ID NO: 56 - DNA, 3 bp, Artificial Sequence
  Synthetic
tgg

SEQ ID NO: 57 - DNA, 6 bp, Artificial Sequence
  Synthetic
gagagt

SEQ ID NO: 58 - DNA, 6 bp, Artificial Sequence
  Synthetic
gggagt

SEQ ID NO: 59 - DNA, 7 bp, Artificial Sequence
  Synthetic
ttgaata

SEQ ID NO: 60 - DNA, 6 bp, Artificial Sequence
  Synthetic
cagagt

SEQ ID NO: 61 - DNA, 6 bp, Artificial Sequence
  Synthetic
tggagt

SEQ ID NO: 62 - DNA, 6 bp, Artificial Sequence
  Synthetic
atgagt

SEQ ID NO: 63 - DNA, 6 bp, Artificial Sequence
  Synthetic
aggaat

SEQ ID NO: 64 - DNA, 22 bp, Artificial Sequence
  Synthetic
aaagatatataatgtcatgaat

SEQ ID NO: 65 - DNA, 22 bp, Artificial Sequence
  Synthetic
gcagaatcaaatataatagtct

SEQ ID NO: 66 - DNA, 22 bp, Artificial Sequence
  Synthetic
caattaaatttgacttattgtt

SEQ ID NO: 67 - DNA, 22 bp, Artificial Sequence
  Synthetic
ctagaccatttcccaccagttc

SEQ ID NO: 68 - DNA, 22 bp, Artificial Sequence
  Synthetic
aggacttttatttaccaaagga

SEQ ID NO: 69 - DNA, 22 bp, Artificial Sequence
  Synthetic
atccaagtccatttgattccta

SEQ ID NO: 70 - DNA, 22 bp, Artificial Sequence
  Synthetic
taattctttctagaaagagcct

SEQ ID NO: 71 - DNA, 22 bp, Artificial Sequence
  Synthetic
ggacatgtgcaagatgcaagag

SEQ ID NO: 72 - DNA, 22 bp, Artificial Sequence
  Synthetic
tgtatgtagaagacctctaagt

SEQ ID NO: 73 - DNA, 22 bp, Artificial Sequence
  Synthetic
tcccctcaccactcacctctga

SEQ ID NO: 74 - DNA, 22 bp, Artificial Sequence
  Synthetic
ctctgataacccagctgtgtgt

SEQ ID NO: 75 - DNA, 19 bp, Artificial Sequence
  Synthetic
ctctgataacccagctgtg

SEQ ID NO: 76 - DNA, 20 bp, Artificial Sequence
  Synthetic
ctctgataacccagctgtgt

SEQ ID NO: 77 - DNA, 21 bp, Artificial Sequence
  Synthetic
ctctgataacccagctgtgtg

SEQ ID NO: 78 - DNA, 23 bp, Artificial Sequence
  Synthetic
ctctgataacccagctgtgtgtt

SEQ ID NO: 79 - DNA, 19 bp, Artificial Sequence
  Synthetic
ctagaccatttcccaccag

SEQ ID NO: 80 - DNA, 20 bp, Artificial Sequence
  Synthetic
ctagaccatttcccaccagt

SEQ ID NO: 81 - DNA, 21 bp, Artificial Sequence
  Synthetic
ctagaccatttcccaccagtt

SEQ ID NO: 82 - DNA, 23 bp, Artificial Sequence
  Synthetic
ctagaccatttcccaccagttct

SEQ ID NO: 83 - DNA, 7009 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 84 - DNA, 7048 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 85 - DNA, 48 bp, Artificial Sequence
  Synthetic
aaaaggccggcggccacgaaaaaggccggccaggcaaaaaagaaaaag

SEQ ID NO: 86 - DNA, 3223 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttatcgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgctttttttccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 87 - DNA, 20 bp, Artificial Sequence
  Synthetic
ctccggaatgtctccatttg

SEQ ID NO: 88 - DNA, 20 bp, Artificial Sequence
  Synthetic
atgagggagagactggcatc

SEQ ID NO: 89 - DNA, 23 bp, Artificial Sequence
  Synthetic
gtttccagagctttacctgagaa

SEQ ID NO: 90 - DNA, 22 bp, Artificial Sequence
  Synthetic
cttttatgaatgcttctccaag

SEQ ID NO: 91 - DNA, 21 bp, Artificial Sequence
  Synthetic
aagttacttgtccaggcatga

SEQ ID NO: 92 - DNA, 25 bp, Artificial Sequence
  Synthetic
gaaaaacttctgccaacttttatca

SEQ ID NO: 93 - DNA, 25 bp, Artificial Sequence
  Synthetic
tgcaaataacaaaagtagccataca

SEQ ID NO: 94 - DNA, 23 bp, Artificial Sequence
  Synthetic
tctttagaaaggcttgaaagctg

SEQ ID NO: 95 - DNA, 20 bp, Artificial Sequence
  Synthetic
cttcactgctggccagttta

SEQ ID NO: 96 - DNA, 21 bp, Artificial Sequence
  Synthetic
ttgaacatggcattgcataaa

SEQ ID NO: 97 - DNA, 20 bp, Artificial Sequence
  Synthetic
cgggcttggacagaacttac

SEQ ID NO: 98 - DNA, 20 bp, Artificial Sequence
  Synthetic
ctgcgtagtgccaaaacaaa

SEQ ID NO: 99 - DNA, 22 bp, Artificial Sequence
  Synthetic
gagatgtcttttgcagctttcc

SEQ ID NO: 100 - DNA, 20 bp, Artificial Sequence
  Synthetic
gggaccttggtaaagccaca

SEQ ID NO: 101 - DNA, 21 bp, Artificial Sequence
  Synthetic
tgcctttcaatcattgtttcg

SEQ ID NO: 102 - DNA, 20 bp, Artificial Sequence
  Synthetic
agaaggcaaattggcacaga

SEQ ID NO: 103 - DNA, 20 bp, Artificial Sequence
  Synthetic
aaggccccaaaatgtgaaat

SEQ ID NO: 104 - DNA, 25 bp, Artificial Sequence
  Synthetic
gagtttggctcaaattgttactctt

SEQ ID NO: 105 - DNA, 18 bp, Artificial Sequence
  Synthetic
tggcggcgttttcattat

SEQ ID NO: 106 - DNA, 26 bp, Artificial Sequence
  Synthetic
ttcgatccgtaatgattgttctagcc

SEQ ID NO: 107 - DNA, 20 bp, Artificial Sequence
  Synthetic
ttgtgtgtcccatgcttgtt

SEQ ID NO: 108 - DNA, 20 bp, Artificial Sequence
  Synthetic
caacgctgaagaaccctgat

SEQ ID NO: 109 - DNA, 24 bp, Artificial Sequence
  Synthetic
tttctgtgattttcttttggattg

SEQ ID NO: 110 - DNA, 20 bp, Artificial Sequence
  Synthetic
gattggctttgatttcccta

SEQ ID NO: 111 - DNA, 20 bp, Artificial Sequence
  Synthetic
gcagttgcctaagaactggt

SEQ ID NO: 112 - RNA, 22 nt, Artificial Sequence
  Synthetic
aaagauauauaaugucaugaau

SEQ ID NO: 113 - RNA, 22 nt, Artificial Sequence
  Synthetic
caauuaaauuugacuuauuguu

SEQ ID NO: 114 - RNA, 22 nt, Artificial Sequence
  Synthetic
uaauucuuucuagaaagagccu

SEQ ID NO: 115 - RNA, 22 nt, Artificial Sequence
  Synthetic
ggacaugugcaagaugcaagag

SEQ ID NO: 116 - RNA, 22 nt, Artificial Sequence
  Synthetic
uguauguagaagaccucuaagu

SEQ ID NO: 117 - RNA, 22 nt, Artificial Sequence
  Synthetic
uccccucaccacucaccucuga

SEQ ID NO: 118 - RNA, 22 nt, Artificial Sequence
  Synthetic
cucugauaacccagcugugugu

SEQ ID NO: 119 - RNA, 19 nt, Artificial Sequence
  Synthetic
cucugauaacccagcugug

SEQ ID NO: 120 - RNA, 20 nt, Artificial Sequence
  Synthetic
cucugauaacccagcugugu

SEQ ID NO: 121 - RNA, 21 nt, Artificial Sequence
  Synthetic
cucugauaacccagcugugug

SEQ ID NO: 122 - RNA, 23 nt, Artificial Sequence
  Synthetic
cucugauaacccagcuguguguu

SEQ ID NO: 123 - RNA, 20 nt, Artificial Sequence
  Synthetic
aacaaauaucccuuaguauc

SEQ ID NO: 124 - RNA, 20 nt, Artificial Sequence
  Synthetic
gauuggcuuugauuucccua

SEQ ID NO: 125 - RNA, 22 nt, Artificial Sequence
  Synthetic
gcagaaucaaauauaauagucu

SEQ ID NO: 126 - RNA, 22 nt, Artificial Sequence
  Synthetic
cuagaccauuucccaccaguuc

SEQ ID NO: 127 - RNA, 22 nt, Artificial Sequence
  Synthetic
aggacuuuuauuuaccaaagga

SEQ ID NO: 128 - RNA, 22 nt, Artificial Sequence
  Synthetic
auccaaguccauuugauuccua

SEQ ID NO: 129 - RNA, 19 nt, Artificial Sequence
  Synthetic
cuagaccauuucccaccag

SEQ ID NO: 130 - RNA, 20 nt, Artificial Sequence
  Synthetic
cuagaccauuucccaccagu

SEQ ID NO: 131 - RNA, 21 nt, Artificial Sequence
  Synthetic
cuagaccauuucccaccaguu

SEQ ID NO: 132 - RNA, 23 nt, Artificial Sequence
  Synthetic
cuagaccauuucccaccaguucu

SEQ ID NO: 133 - RNA, 20 nt, Artificial Sequence
  Synthetic
aauguauuucuucuauucaa

SEQ ID NO: 134 - RNA, 20 nt, Artificial Sequence
  Synthetic
gcaguugccuaagaacuggu